(19) United States
(12) Patent Application Publication
(10) Pub. No.: US 2020/0129582 A1
(43) Pub. Date: Apr. 30, 2020

(54) NOVEL PEPTIDES AND PEPTIDOMIMETICS

(71) Applicants: UNIVERSITY OF AUCKLAND, Auckland (NZ); OCUNEXUS THERAPEUTICS, INC., San Diego, CA (US)

(72) Inventors: Colin Richard GREEN, Auckland (NZ); Yeri KIM, North Shore City (NZ); Bradford James DUFT, Solana Beach, CA (US)

(21) Appl. No.: 16/669,217

(22) Filed: Oct. 30, 2019

Related U.S. Application Data
(63) Continuation of application No. 15/585,082, filed on May 2, 2017, now abandoned.
(60) Provisional application No. 62/331,416, filed on May 3, 2016.

(51) Int. Cl. A61K 38/02; G01N 33/50; C07K 14/00; C07K 14/705

(52) U.S. Cl. CPC A61K 38/02; G01N 33/5008; A61K 38/00; C07K 14/705; G01N 2800/32; C07K 14/00

(57) ABSTRACT

Compounds and compositions comprising a gap junction channel or hemichannel blocker or inhibitor, and methods of use thereof, are provided for the treatment or prevention of vascular and other diseases, disorders, and conditions

### RELATED APPLICATION

This application is a Continuation of U.S. application Ser. No. 15/585,082 filed on May 2, 2017, which claims priority to U.S. Provisional Application No. 62/331,416, entitled NOVEL PEPTIDES AND PEPTIDOMIMETICS, filed May 3, 2016, the contents of which are herein incorporated by reference as if set forth in their entirety.

### FIELD

The inventions relate to petides and peptidomimetics.

### BACKGROUND

The following includes information that may be useful in understanding the present invention. It is not an admission that any of the information, publications or documents specifically or implicitly mentioned or referenced herein is prior art, or essential, to the presently described or claimed inventions.

All U.S. patents, U.S. patent application publications, foreign patents, foreign and PCT published applications, articles and other documents, references and publications noted herein, and all those listed as References Cited in any patent or patents that issue herefrom, are hereby incorporated by reference in their entirety.

Gap junctions are cell membrane structures that facilitate cell-cell communication. They consist of clusters of intercellular channels that directly connect the cytoplasm of two adjacent cells and allow various molecules, ions, and electrical impulses to directly pass between them. Gap junction channels are permeable to substances with a molecular weight of <≈1 kDa. Permeability depends on connexin type and charge of the permeating molecule. These channels are found in nearly all animal cells that touch each other, and are expressed in virtually all tissues of the body, with the exception of mature skeletal muscle and mobile cell types such as sperm and erythrocytes. They appear as clusters (or plaques) of tightly packed particles, in which each particle is a single gap junction channel. There can be many gap junction plaques per cell, with each plaque containing a few to thousands of intercellular channel units.

Intercellular gap junction channels are formed by head-to-head docking of two half-channels, called “connexons” or “hemichannels,” with a hemichannel in one cell membrane docking with another hemichannel in an opposing membrane to form a single gap junction channel. Each gap junction hemichannel is an assembly of six tetraspan integral membrane proteins, called “connexins,” hexagonally arranged around an aqueous pore. All connexin proteins consist of four highly conserved a-helical membrane-spanning segments separated by two extracellular and one intracellular loop. The amino and carboxy terminals are located intracellularly. The extracellular loops recognize and discriminate adjacent connexin extracellular loops, and can form an impermeable seal between the channel lumen and the extracellular space (White T W, et al. (1994) Journal of Cell Biology 125:879-892). Twenty-one members of the human connexin family have been identified. They differ mainly in the sequence of their intracellular loops and carboxy termini, with each connexin having a name based on its putative molecular mass in kilodaltons. Connexin26, for example, is a 26 kD protein.

In addion to faclitiating cell-cell communication, the connexin43 gap junction protein that also appears to play an important role in acute injury response and in some chronic diseases, with protein expression going down in the former and up in the latter. Gap junction hemichannels may also be implicated. Prior to docking with a neighboring cell, connexin43 hemichannels are reported by some to have a low open probability as open channels constitute a large, relatively non-specific membrane pore, and while connexin43 hemichannels are said to be tightly controlled under resting conditions (Bukauskas F F, et al. (2000) Proceedings of the National Academy of Sciences 97:2556-2561; Contreras J E, et al. (2003) Proceedings of the National Academy of Sciences 100:11388-11393), they have also been reported to open in response to a number of factors characteristic of injury including pro-inflammatory cytokines (Retamal M A, et al. (2007) Journal of the Society for Neuroscience 27:13781-13792), metabolic inhibition (John S A, et al. (1999) Journal of Biological Chemistry 274:236-240; Contreras J E, et al. (2002) Proceedings of the National Academy of Sciences 99:495-500), Ca²⁺ concentration (increase in cytoplasmic Ca²⁺ concentration or decrease in extracellular concentration) (Li H, et al. (1996) Journal of Cell Biology 134:1019-1030; Thimm J, et al. (2005) Journal of Biological Chemistry 280:10646-10654; De Vuyst E, et al. (2009) Cell Calcium 46:176-187) and by infectious materials such as gram-positive bacterial cell wall proteins (peptidoglycan or lip polysaccharide) (Robertson J, et al. (2010) Biochemical Journal 432:133-143). Gap junction channels and proteins have been discussed with regard to various pathologies including spinal cord injury (O'Carroll S J, et al. (2008) Cell Communication & Adhesion 15:27-42; O'Carroll S J, et al. (2013) Neuroscience research 75:256-267; Tonkin R S, et al. (2014) Frontiers in Molecular Neuroscience 7:102), preterm ischemia and asphyxiation (Davidson J O, et al. (2012) Annals of Neurology 71:121-132; Davidson J O, et al. (2014) PloS One 9:e96558), infection (Eugenin E A, et al. (2011) The Journal of Neuroscience 31:9456-9465), cerebral malaria (Zhang J, et al. (2013) Connexin-Based Therapeutic Approaches to Inflammation in the Central Nervous System. In: Connexin Cell Communication Channels (Oviedo-Orta, E. et al., eds), pp 273-306: CRC Press), and retinal ischemia-reperfusion injury (Danesh-Meyer H V, et al. (2012) Brain 135:506-520). Hemichannel opening may also have a role in chronic inflammation, reportedly through the inflammasome pathway (Kim Y, et al. (2016) Adv Protein Chem Struct Biol 104:1-37). For review see Danesh-Meyer H V, et al. (2015) Prog Retin Eye Res. 51:41-68 (connexin43 upregulation and hemichannel opening implicated in various aspects of secondary damage, including glial cell activation, edema and loss of vascular integrity). See also Vinken, M (2015) Arch Toxicol. 89(1): 143-145. Thus, while gap junctions allow for movement of ions and metabolites between cells that is crucial for cell survival (Krysko D V, et al. (2005) Apoptosis 10:459-469), opening of a large, relatively non-selective, undocked hemichannel pore has a capacity to drive cell death by degrading ionic and metabolic gradients at the lesion site (Takeuchi H, et al. (2006) Journal of Biological Chemistry 281:21362-21368) compromising the ability of cells to osmoregulate (Quist A P, et al. (2000) Journal of Cell Biology 148:1063-1074; Rodriguez-Sinovas A, et al. (2007) Progress in Biophysics and Molecular Biology 94:219-232; Danesh-Meyer H V, et al. (2012) Brain 135:506-520) and promoting the spread of secondary damage Davidson J O, et al. (2013a) Current Drug Targets 14:36-46; Decrock E, et al. (2015) Cellular and Molecular Life Sciences 72:2823-2851.

Small compounds such as alcohols (heptanol, octanol), anaesthetics (halothane and ethane) fatty acids (oleamide), anti-malarial drugs (quinine derivatives), glycyrrhetinic acid and its derivative, carbenoxolone, have been shown to block connexin channels, but these compounds are limited in their therapeutic application owing to off-target effects. For review see Bodendiek S B and Raman G (2010) Current Medicinal Chemistry 17:4191-4230. Other more specific anti-connexin approaches have also been tested. For example, connexin43 oligodeoxynucleotide antisense has been topically applied to reduce swelling, inflammation and improve functional outcomes in skin lesions on mice (Qiu, C. et al., (2003) Current Biology, 13:1967-1703), neonatal mouse burns (Coutinho P, et al. British Journal of Plastic Surgery 58:658-667), and rodent spinal cord injuries, Cronin M, et al. (2008) Molecular and Cellular Neurosciences 39:152-160). Connexin43 antisense has demonstrated efficacy in non-healing ocular burns in compassionate use cases (Ormonde S, et al. (2012) Journal of Membrane Biology 245:381-388). Other approaches have focused on peptides matching portions of connexin extracellular loops (such as for example Gap26, Gap27, Peptide5) (Evans W H (2015) Biochem Soc Trans 43:450-459) or intracellular regions of the connexin43 protein (for example Gap19, ACT-1) (D'Hondt C, et al. (2013) Biochem Biophys Res Commun 432:707-712). However, the latter two peptides need to cross the cell membrane if they are to be effective. Gap26 and Gap27 are hypothesized to act on connexin43 hemichannel gating (reviewed in Wang N, De Bock M, Decrock E, Bol M, Gadicherla A, Bultynck G, Leybaert L (2013) Connexin targeting peptides as inhibitors of voltage- and intracellular Ca2+-triggered Cx43 hemichannel opening. Neuropharmacology 75:506-516). Peptide5 (VDCLSRPTEKT SEQ ID NO: 72)) was derived from the extracellular loop 2 sequence of connexin43 and introduced by O'Carroll et al. (2008), supra, and is reported to have a concentration-dependent action. See Davidson J O, et al. (2012) supra; Davidson J O, et al. (2014), supra; Danesh-Meyer H V, et al. (2012) supra.

There remains a need in the art for new therapeutics useful in treating diseases, disorders and conditions, including ocular and CNS diseases, disorders and conditions, that can be ameliorated by hemichannel and gap junction modulation. There is a particular need for new therapeutics that span the entire spectrum of vascular diseases, disorders and conditions associated with inflammation, edema and/or oxidative stress. Such therapeutics are described and claimed herein, based on the discoveries of new gap junction and hemichannel modulating agents.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

The present invention provides for the production of peptides and peptide mimetics having pharmacokinetic and pharmacodynamic properties useful for modulating gap junction communication and hemichannel opening and other activities described herein, and their use in the treatment of diseases, disorders and conditions where modulation of gap junction activity and/or hemichannel opening and hemichannel activity is therapeutically beneficial.

The inventions described and claimed herein relate to peptides and peptidomimetics that bind to gap junction and gap junction hemichannel proteins. Applicants' inventions also relate to pharmaceuticals, compositions and methods useful for treating, preventing and ameliorating the effects of vascular diseases, disorders and conditions, as well as articles and kits comprising such compounds and compositions. In one aspect, the inventions relate to compounds for controlling gap junction protein channel activity. In another aspect, the inventions relate to compounds to control gap junction hemichannel activity.

In one embodiment, the peptides and peptide mimetics described herein are gap junction channel inhibitors. In one embodiment, the gap junction comprises a connexin43 protein. In one embodiment, the gap junction comprises a connexin45 protein. In another embodiment, the peptides and peptide mimetics described herein are inhibitors of gap junction hemichannel opening. In one embodiment, the gap junction hemichannel comprises a connexin43 protein. In one embodiment, the gap junction hemichannel comprises a connexin45 protein. In other embodiments, the gap junctions and hemichannels comprise connexin25, connexin26, connexin29 (connexin30.2), connexin30, connexin30.3, connexin31, connexin31.1, connexin31.3, connexin31.9, connexin32, connexin36, connexin37, connexin40, connexin40.1, connexin46, connexin46.6 (connexin47), connexin50, connexin58, connexin59, connexin62 proteins, and the peptides and peptide mimetics described herein are inhibitors of one or more of these gap junction channels and corresponding hemichannels.

In another aspect, the inventions relate to compounds to control the opening of surface-exposed gap junction hemichannels. In another aspect, the inventions relate to compounds for sustained control of hemichannel opening. In another aspect, the inventions relate to compounds for closing hemichannels. In yet another aspect, the inventions relate to compounds for modulating hemichannel opening.

In one aspect, the inventions provided herein include compounds. In another aspect, the inventions include compositions comprising or consisting essentially of one or more of those compounds, the basic and novel characteristic of the inventions being the ability to modulate gap junction and/or hemichannel function or activity as described herein. The compounds are useful for the treatment of gap junction- and/or hemichannel-related disorders.

Compounds of the invention, which in a non-limiting preferred embodiment are isolated or substantially pure, include the following peptides: “Ala1” ADCFLSRPTEKT (SEQ.ID.NO:1), “Ala4” VDCALSRPTEKT (SEQ.ID.NO:2), “Ala6” VDCFLARPTEKT (SEQ.ID.NO:3), “Ala9” VDCFLSRPAEKT (SEQ.ID.NO:4), “Ala12” VDCFLSRPTEKA (SEQ.ID.NO:5), “Mod-1/Ala10” CFLSRPTAKT (SEQ.ID.NO:6), “Mod-1/Ala4” CALSRPTEKT (SEQ.ID.NO:7), “Mod-1/Ala6” CFLARPTEKT (SEQ.ID.NO:8), “Mod-1/Ala9” CFLSRPAEKT (SEQ.ID.NO:9), and “Mod-1/Ala12” CFLSRPTEKA (SEQ.ID.NO:10). In any and all of the above peptides shown as SEQ.ID.NO:1-10, separately or together in any combination, Valine (V) can be substituted with Alanine (A), Isoleucine (I), Leucine (L), Methionine (M) and Phenylalanine (F); Aspartic Acid (Asp) can be substituted with Glutamic Acid (E); Cysteine (C) can be substituted with Serine (S); Phenylalanine (P) can be substituted with Alanine (A), Tyrosine (Y), Tryptophan (W), Leucine (L), Isoleucine (I) and Valine (V); Leucine (L) can be substituted with Isoleucine (I), Methionine (M) and Valine (V); Serine (S) can be substituted with Alanine (A), Isoleucine (I), Leucine (L) and Valine (V), preferably Alanine; Arginine (R) can be substituted with Lysine (L) or Glutamine (Q); Proline (P) cannot be substituted; Threonine (T) can be substituted with Alanine (A), Asparagine (N), Serine (S) and Glysine (G); Glutamic Acid (E) can be substituted with Alanine (A), Glutamine (Q) and Asparagine (N); Lysine (K) can be substituted with Arginine (R), all of which are SEQ.ID.NO:1-8 analogs. In any and all of the above peptides shown as SEQ.ID.NO:1-8, separately or together in any combination, any of the amino acids can be L-amino acids or D-amino acids, provided, however, that the peptide is not L-Val L-Asp L-Cys L-Phe L-Leu L-Ser L-Arg L-Pro L-Thr L-Glu L-Lys L-Thr (SEQ ID NO: 45). Any of the above peptides shown as SEQ.ID.NO:1-8 that contain D-amino acids are also SEQ.ID.NO:1-8 analogs.

Compounds of the invention, which in a non-limiting preferred embodiment are isolated or substantially pure, also include compounds according to the following Formula I:

R₁ Val Asp Cys X₁ Leu Ser Arg Pro X₂ X₃ Lys X₄ (SEQ ID NO: 73)

wherein R₁ is absent, or selected from the group consisting of H, acyl groups containing a linear or branched, saturated or unsaturated hydrocarbon chain from 1 to 20 carbon atoms, amides, carbamates, ureas, PEG, or hydroxyalkyl starch; X₁ is Phe, or an amino acid selected from the group consisting of Ala, Ile, Leu, Val, dehydroalanine, aminoisobutyric acid, and/or Trp, Tyr, Met, Ile, Leu, and/or Ser, Asn, Cys, Gln, Thr, Asp, Glu, Arg, His, Lys, Pro, and/or Nva or Nle, and/or tert-butylglycine, phenylglycine, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, and N-methyl-lysine, and/or a D-enantiomer of any of the foregoing; X₂ is Thr, or an amino acid selected from the group consisting of Ser, Asn, Ala, Be, Leu, Val, dehydroalanine, aminoisobutyric acid, Asp, Glu, Lys, Met, His, Pro, Gln, Arg, Ser, Nva or Nle; X₃ is Glu, Ala, dehydroalanine, aminoisobutyric acid, Asp, Lys, Gln, Gly, His, Arg, Asn, Met, Pro, Ser, Thr, Nva or Nle; and X₄ is Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle; provided, however, X₁, X₂, X₃ and X₄ are not, respectively, Phe, Thr, Glu and Thr at the same time and the peptide R₁ Val Asp Cys X₁ Leu Ser Arg Pro X₂ X₃ Lys X₄ peptide (SEQ ID NO: 73) is not L-Val L-Asp L-Cys L-Phe L-Leu L-Ser L-Arg L-Pro L-Thr L-Glu L-Lys L-Thr (SEQ ID NO: 45).

The invention also includes compounds of Formula I wherein X₁ is Phe and one or more of X₂, X₃ and/or X₄ are not, respectively, Thr, Glu and Thr. The invention also includes compounds of Formula I wherein X₂ is Thr and one or more of X₁, X₃ and/or X₄ are not, respectively, Phe, Glu and Thr. The invention also includes compounds of Formula I wherein X₃ is Glu and one or more of X₁, X₂ and/or X₄ are not, respectively, Phe, Thr and Thr. The invention also includes compounds of Formula I wherein X₄ is Thr and one or more of X₁, X₂ and/or X₃ are not, respectively, Phe, Thr and Glu.

Compounds of the invention, which in a non-limiting preferred embodiment are isolated or substantially pure, also include compounds according to the following Formula II:

R₁ Y₁ Asp Cys X₁ Leu Z₁ Arg Pro X₂ X₃ Lys X₄ (SEQ ID NO: 74)

Wherein Y₁ is Val, Ala or an amino acid selected from the group consisting of Ile, Leu, Met, Phe, Thr, Cys, Tyr, dehydroalanine, aminoisobutyric acid, Nva, Nle, tert-butylglycine, phenylglycine, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, and N-methyl-lysine, and/or a D-enantiomer of any of the foregoing; Z₁ is Ser, Ala or an amino acid selected from the group consisting of Asn, Thr, Cys, Gly, Asp, Glu, Lys, Gln, Gly, Pro, His and Arg; and wherein R₁, X₁, X₂, X₃ and X₄ are as described above for Fomula I; provided, however, that Y₁, X₁, Z₁, X₂, X₃ and X₄ are not, respectively, Val, Phe, Ser, Thr, Glu and Thr at the same time and the peptide R₁ Y₁ Asp Cys X₁ Leu Z₁ Arg Pro X₂ X₃ Lys X₄ peptide (SEQ ID NO: 74) is not L-Val L-Asp L-Cys L-Phe L-Leu L-Ser L-Arg L-Pro L-Thr L-Glu L-Lys L-Thr (SEQ ID NO: 45).

The invention also includes compounds of Formula II wherein Y₁ is Val, and one or more of X₁, Z₁, X₂, X₃ and/or X₄ are not, respectively, Phe, Ser, Thr, Glu and Thr. The invention also includes compounds of Formula I wherein X₁ is Phe and one or more of Y₁, Z₁, X₂, X₃ and/or X₄ are not, respectively, Val, Ser, Thr Glu and Thr. The invention also includes compounds of Formula I wherein X₂ is Thr and one or more of Y₁, X₁, Z₁, X₃ and/or X₄ are not, respectively, Val, Phe, Ser, Glu and Thr. The invention also includes compounds of Formula I wherein X₃ is Glu and one or more of Y₁, X₁, Z₁, X₂ and/or X₄ are not, respectively, Val, Phe, Ser, Thr and Thr. The invention also includes compounds of Formula I wherein X₄ is Thr and one or more of Y₁, X₁, Z₁, X₂ and/or X₃ are not, respectively, Val, Phe, Ser, Thr and Glu.

Compounds of the invention, which in a non-limiting preferred embodiment are isolated or substantially pure, also include compounds according to the following Formula III:

R₁ Ile Asp Cys X₁ Ile Ser Arg Pro X₂ X₃ Lys X₄ (SEQ ID NO: 75)

wherein R₁ is absent, or selected from the group consisting of H, acyl groups containing a linear or branched, saturated or unsaturated hydrocarbon chain from 1 to 20 carbon atoms, amides, carbamates, ureas, PEG, or hydroxyalkyl starch; X₁ is Phe, or an amino acid selected from the group consisting of Ala, Ile, Leu, Val, dehydroalanine, aminoisobutyric acid, and/or Trp, Tyr, Met, Ile, Leu, and/or Ser, Asn, Cys, Gln, Thr, Asp, Glu, Arg, His, Lys, Pro, and/or Nva or Nle, and/or tert-butylglycine, phenylglycine, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, and N-methyl-lysine, and/or a D-enantiomer of any of the foregoing; X₂ is Thr, or an amino acid selected from the group consisting of Ser, Asn, Ala, Be, Leu, Val, dehydroalanine, aminoisobutyric acid, Asp, Glu, Lys, Met, His, Pro, Gln, Arg, Ser, Nva or Nle; X₃ is Glu, Ala, dehydroalanine, aminoisobutyric acid, Asp, Lys, Gln, Gly, His, Arg, Asn, Met, Pro, Ser, Thr, Nva or Nle; and X₄ is Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle; provided, however, X₁, X₂, X₃ and X₄ are not, respectively, Phe, Thr, Glu and Thr at the same time and the peptide R₁ Ile Asp Cys X₁ Ile Ser Arg Pro X₂ X₃ Lys X₄ peptide (SEQ ID NO: 75) is not L-Ile L-Asp L-Cys L-Phe L-Ile L-Ser L-Arg L-Pro L-Thr L-Glu L-Lys L-Thr (SEQ ID NO: 43), or any naturally occurring connexin peptide sequence.

The invention also includes compounds of Formula III wherein X₁ is Phe and one or more of X₂, X₃ and/or X₄ are not, respectively, Thr, Glu and Thr. The invention also includes compounds of Formula III wherein X₂ is Thr and one or more of X₁, X₃ and/or X₄ are not, respectively, Phe, Glu and Thr. The invention also includes compounds of Formula III wherein X₃ is Glu and one or more of X₁, X₂ and/or X₄ are not, respectively, Phe, Thr and Thr. The invention also includes compounds of Formula III wherein X₄ is Thr and one or more of X₁, X₂ and/or X₃ are not, respectively, Phe, Thr and Glu.

Compounds of the invention, which in a non-limiting preferred embodiment are isolated or substantially pure, also include compounds according to the following Formula IV:

R₁ Y₁ Asp Cys X₁ ILe Z₁ Arg Pro X₂ X₃ Lys X₄ (SEQ ID NO: 76)

Wherein Y₁ is Be, Val, Leu, Ala, Met, and/or Phe or an amino acid selected from the group consisting of Ile, Leu, Met, Phe, Thr, Cys, Tyr, dehydroalanine, aminoisobutyric acid, Nva, Nle, tert-butylglycine, phenylglycine, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, and N-methyl-lysine, and/or a D-enantiomer of any of the foregoing; Z₁ is Ser, Ala or an amino acid selected from the group consisting of Asn, Thr, Cys, Gly, Asp, Glu, Lys, Gln, Gly, Pro, His and Arg; and wherein R₁, X₁, X₂, X₃ and X₄ are as described above for Fomula III; provided, however, that Y₁, X₁, Z₁, X₂, X₃ and X₄ are not, respectively, Val, Phe, Ser, Thr, Glu and Thr at the same time and the peptide R₁ Y₁ Asp Cys X₁ Ile Z₁ Arg Pro X₂ X₃ Lys X₄ peptide (SEQ ID NO: 76) is not L-Ile L-Asp L-Cys L-Phe L-Ile L-Ser L-Arg L-Pro L-Thr L-Glu L-Lys L-Thr (SEQ ID NO: 43), or any naturally occurring connexin peptide sequence.

Compounds of the invention, which in a non-limiting preferred embodiment are isolated or substantially pure, may also include or exclude 12 amino peptides according to the following Table 1:

TABLE 1

| CONNEXIN | | | | | | | | | | | | | |
|---|---|---|---|---|---|---|---|---|---|---|---|---|---|
| ISOFORM | AMINO ACIDS 1-12 | | | | | | | | | | | | SEQ ID NO: |
| Cx46 | v | d | c | f | i | s | r | p | t | e | k | t | SEQ ID NO: 11 |
| Cx50 | v | d | c | f | v | s | r | p | t | e | k | t | SEQ ID NO: 12 |
| Cx40 | v | n | c | y | v | s | r | p | t | e | k | n | SEQ ID NO: 13 |
| Cx59 | i | d | c | f | v | s | r | p | t | e | k | t | SEQ ID NO: 14 |
| Cx62 | v | d | c | f | v | s | r | p | t | e | k | t | SEQ ID NO: 15 |
| Cx37 | v | d | c | f | v | s | r | p | t | e | k | t | SEQ ID NO: 16 |
| Cx46.6 (47) | v | d | c | f | v | s | r | p | t | e | k | t | SEQ ID NO: 17 |
| Cx30.3 | v | d | c | y | i | s | r | p | t | e | k | k | SEQ ID NO: 18 |
| Cx31.1 | v | d | c | f | i | s | k | p | s | e | k | n | SEQ ID NO: 19 |
| Cx31 | v | d | c | y | i | a | r | p | t | e | k | k | SEQ ID NO: 20 |
| Cx25 | v | d | c | f | i | s | k | p | t | e | k | t | SEQ ID NO: 21 |
| Cx26 | v | d | c | f | v | s | r | p | t | e | k | t | SEQ ID NO: 22 |
| Cx30 | v | d | c | f | i | s | r | p | t | e | k | t | SEQ ID NO: 23 |
| Cx32 | v | d | c | f | v | s | r | p | t | e | k | t | SEQ ID NO: 24 |
| Cx36 | v | e | c | y | v | s | r | p | t | e | k | t | SEQ ID NO: 25 |
| Cx31.9 | v | d | c | f | v | s | r | p | t | e | k | t | SEQ ID NO: 26 |
| Cx40.1 | v | d | c | y | v | s | r | p | t | e | k | s | SEQ ID NO: 27 |
| Cx29 (30.2) | i | t | c | n | l | s | r | p | s | e | k | t | SEQ ID NO: 28 |
| Cx31.3 | i | t | c | n | l | s | r | p | s | e | k | t | SEQ ID NO: 29 |
| Cx58 | i | d | c | f | v | s | r | p | t | e | k | t | SEQ ID NO: 30 |

Other embodiments of the invention, which in a non-limiting preferred embodiment are isolated or substantially pure, also include the amino acid sequence VDCFVSRPTEKT (SEQ ID NO:31), which is useful, for example, as a blocker for gap junction channels and/or hemichannels composed of any of connexins 50, 62, 37, 46.6, 26, 32 or 31.9; the amino acid sequence VDCFISRPTEKT (SEQ ID NO:32), which is useful, for example, as a blocker for gap junction channels and/or hemichannels composed of any of connexins 46 or 30.

Other embodiments of the invention, which in a non-limiting preferred embodiment are isolated or substantially pure, also include the amino acid sequence ITCNLSRPSEKT (SEQ ID NO:33), which is useful, for example, as a blocker for gap junction channels and/or hemichannels composed of any of connexins 29 (30.2) or 31.3; the amino acid sequence VDCYVSRPTEKS (SEQ ID NO:34), which is useful, for example, as a blocker for gap junction channels and/or hemichannels composed of connexin40.1; the amino acid sequence VECYVSRPTEKT (SEQ ID NO:35), which is useful, for example, as a blocker for gap junction channels and/or hemichannels composed of connexin36; the amino acid sequence IDCFVSRPTEKT (SEQ ID NO:36), which is useful, for example, as a blocker for gap junction channels and/or hemichannels composed of connexin 59; the amino acid sequence VDCYISRPTEKK (SEQ ID NO:37), which is useful, for example, as a blocker for gap junction channels and/or hemichannels composed of connexin 30.3; the amino acid sequence VDCFISKPSEKN (SEQ ID NO:38), which is useful, for example, as a blocker for gap junction channels and/or hemichannels composed of connexin 31.1; the amino acid sequence VDCYIARPTEKK (SEQ ID NO:39), which is useful, for example, as a blocker for gap junction channels and/or hemichannels composed of connexin 31; the amino acid sequence VDCFISKPTEKT (SEQ ID NO:40), which is useful, for example, as a blocker for gap junction channels and/or hemichannels composed of connexin 25; and, the amino acid sequence IDCFVSRPTEKT (SEQ ID NO:41), which is useful, for example, as a blocker for gap junction channels and/or hemichannels composed of any of connexin58 and connexin59.

Other embodiments of the invention, which in a non-limiting preferred embodiment are isolated or substantially pure, also include the amino acidsequence VNCYVSRPTEKN (SEQ ID NO:42) will be isoform specific and useful, for example, as a blocker for gap junction channels and/or as a hemichannel blocker for hemichannels composed of Connexin40. The sequence and active analogs thereof, including those with groups such as R₁ described above, and others useful for example to increase molecular mass and shield them from proteolytic enzymes, will be particularly important for addressing vascular diseases, disorders and conditions, including those characterized at least in part by microvessel leakage, and as cancer treatments.

Other embodiments of the invention, which in a non-limiting preferred embodiment are isolated or substantially pure, also include the amino acidsequence IDCFISRPTEKT (SEQ ID NO:43) will be isoform specific and useful, for example, as a blocker for gap junction channels and/or as a hemichannel blocker for hemichannels composed of Connexin 45. The sequence and active analogs thereof, including those with groups such as R₁ described above, and others useful for example to increase molecular mass and shield them from proteolytic enzymes, will be particularly important for addressing cardiac diseases, disorders and conditions, including those characterized at least in part by vessel damage and/or leakage, and as treatments to address vessel dieback from myocardial infarction and heart attack.

Other embodiments of the invention include combination treatments with peptides matching regions of both connexin extracellular loops 1 and 2 show increased efficacy (FIG. 3, Table 2), exceeding the efficacy of carbenoxolone commonly used as a gold standard control channel blocker.

Other embodiments of the invention include compositions and treatments comprising a combination of the connexin hemichannel blocker VCYDKSFPISHVR (SEQ ID NO:44) with any one or more of the peptides described herein, including those according to Formula I or Formula II, will also be useful as a connexin43 hemichannel blocking combination.

Other embodiments of the invention include compositions and treatments comprising a combination of the connexin hemichannel blocker VCYDKSFPISHVR (SEQ ID NO:44) with any one or more of the peptides described herein, including those according to Formula III or Formula IV, will also be useful as a connexin hemichannel blocking combination.

These sequences may be delivered separately or together as independent entities or linked. Thus, the invention also includes, for example, the connexin43 hemichannel blocker VCYDKSFPISHVR (SEQ ID NO:44) linked to the connexin43 hemichannel blocker VDCFLSRPTEKT (SEQ ID NO:45) in the form, for example, VDCFLSRPTEKT-X-VCYDKSFPISHVR (SEQ ID NO:46) where X is one or more linking glycines or other non-functional linker with sufficient flexibility for the combination peptide to interact with both of their binding sites respectively on connexin43 hemichannels.

The invention also includes, for example, sequences in combination that are functional against multiple connexin isoforms in a tissue at the same time, and may be delivered together separately or together as independent entities or linked. For example, the connexin43 hemichannel blocker VDCFLSRPTEKT (SEQ ID NO:47) can be linked to the connexin40 hemichannel blocker VNCYVSRPTEKN (SEQ ID NO:48) in the form VDCFLSRPTEKT-X-VNCYVSRPTEKN (SEQ ID NO:49) where X is one or more linking glycines or other non-functional linker enabling the combination peptide to interact with hemichannels composed of either Connexin43 or Connexin40.

The invention also includes, for example, the or four peptide connexin isoform blocker sequences in combination, and which do not form tertiary structures and the linking provides for retention of linear structure. In one embodiment, a peptide for vascular endothelial connexins 40, 43 and 37 is prepared, including: VDCFVSRPTEKN (SEQ ID NO:50); VDCFLSRPTEKN (SEQ ID NO:51); and VDCFVSRPTEKT (SEQ ID NO:52).

Included in the scope of the invention are active analogs and conservative variants of these compounds, including truncations thereof, preferably N-terminal truncations of 1 or 2 amino acids.

In one non-limiting embodiment, one or more of the amino acids of the peptides within the scope of the invention, including SEQ.ID.NOS:1-52 and sequences within Formulae I-II, may be in the L- or D-configuration. In other embodiments, one or more of the amino acids of the peptides within the scope of the invention are naturally-occuring non-genetically coded amino acids. In still other embodiments, one or more of the amino acids of the peptides within the scope of the invention are amino acid analogs or synthetic amino acids.

In another non-limiting embodiment, the N-terminal amino acid may be modified to contain a formyl group, a group comprising a formyl group, an ester of a carboxylic acid (preferably an aldehyde ester, e.g., a carboxyethyl group, a carboxymethyl group, etc.), or a group comprising an ester of a carboxylic acid. Modifications with formyl, carboxyethyl, and carboxymethyl groups are presently preferred.

In another embodiment, one or more the amino acids in compounds within the scope of the invention, including SEQ.ID.NOS:1-52 and sequences within Formulae I-II, are substituted for another amino acid from a similar amino acid class or subclass, based primarily upon the chemical and physical properties of the amino acid side chain. For example, one or more hydrophilic or polar amino acids can be substituted for another hydrophilic or polar amino acid. Likewise, one or more hydrophobic or nonpolar amino acids can be substituted for another hydrophobic or nonpolar amino acid. In making such substitutions, polar amino acids can be further subdivided into amino acids having acidic, basic or hydrophilic side chains and nonpolar amino acids can be further subdivided amino acids having aromatic or hydrophobic side chains. Nonpolar amino acids may be further subdivided to include, among others, aliphatic amino acids.

Also within the scope of the invention are compounds of the invention that have been modified to improve their biopharmaceutical properties. In certain embodiments, the compounds of the invention are modified, for example, to provide increased stability, increased resistance to proteolytic inactivation, decreased to nonexistent immunogenicity, increased circulatory lives, including modified serum half-lives and modified therapeutic half-lives, and low toxicity. Modified forms of compounds of the invention include prodrug forms, representative examples of which are described elsewhere herein. Methods by which the compounds of the invention can be modified also include, for example, by PEGylation, by chemical derivitization, and by fusion or conjugation with peptides or lipids. Modifided compounds include modified peptide hemichannel agents, including, for example, any of modified SEQ ID NOS:1-10, and modified analogs, and variants (e.g., conservative variants) thereof.

Other embodiments include peptides selected from SEQ.ID.NOS:1 to 10, and peptides according to Formula I and/or Formula II, that do not include a C-terminal Threonine. Other embodiments include peptides selected from SEQ.ID.NOS:1 to 10, and peptides according to Formula I and/or Formula II, that do not include an N-terminal Valine. Other embodiments include peptides selected from SEQ.ID.NOS:1 to 10, and peptides according to Formula I and/or Formula II, that do not include an N-terminal Valine or a C-terminal Threonine.

Other embodiments include peptides selected from SEQ.ID.NOS:11 to 52 that have the C-terminal amino acid removed, i.e., a single C-terminal truncation. Other embodiments include peptides selected from SEQ.ID.NOS:11 to 52 that have the first and/or second N-terminal amino acid removed, i.e., a single or double N-terminal truncation.

In some embodiments, present invention provides a pharmaceutical composition comprising one or more of the peptides or peptide mimetics of the present invention, and a pharmaceutically acceptable carrier or excipient. In some embodiments, present invention provides a kit for the prophylaxis or treatment of a mammal for one or more of the diseases, disorders or conditions described or referenced herein, characterized in that said kit comprises one or more gap junction channel and/or hemichannel blockers or inhibitors, optionally with reagents and/or instructions for use, wherein said one or more gap junction channel and/or hemichannel blockers or inhibitors comprise a sequence of at least 10 contiguous amino acids of any of the peptides or peptide mimetics described herein.

The present inventions also include pharmaceutical compositions comprising or consisting essentially of the peptide fragment agent and a pharmaceutically acceptable carrier and one or more of the gap junction channel and/or hemichannel blockers or inhibitor compounds described or referenced herein. In one embodiment, the pharmaceutical composition comprises or consists essentially of any of SEQ ID NOS:1-52. In another embodiment, the pharmaceutical composition comprises or consists essentially of a sequence selected from either of Formula I or Formula II. Included in the scope of the invention are pharmaceutical compositions including one or more active analogs and conservative variants of these compounds, including truncations thereof, preferably 1- or 2-amino acid N-terminal truncations as described, or a 1-amino acid C-terminal truncation.

In another embodiment, the inventions include pharmaceutical compositions comprising or consisting essentially of compounds of the invention, including analogs, variants, truncations, etc., that have been modified to improve their biopharmaceutical properties. In certain embodiments, the compounds of the invention are modified, for example, to provide increased stability, increased resistance to proteolytic inactivation, decreased to nonexistent immunogenicity, increased half-lives or circulatory lives, and low toxicity. Methods by which the compounds of the invention can be modified include, for example, by PEGylation, by chemical derivitization, and by fusion or conjugation with peptides or lipids.

The inventions include a pharmaceutical composition comprising one or more pharmaceutically acceptable gap junction channel and/or hemichannel blockers or inhibitor compounds described or referenced herein for the treatment of a cardiovascular disorder, e.g., an acute coronary syndrome, heart failure, ischemic heart disease, etc., and related cardiovascular diseases, disorders and conditions characterized at least in party by ischemia and/or oxidative stress, and related disorders and conditions. Certain preferred gap junction channel and/or hemichannel blockers or inhibitor compounds are identified herein as SEQ.ID.NOS:1 to 52. SEQ.ID.NOS:1-10 are particularly preferred connexin43 gap junction channel and/or hemichannel blockers or inhibitor compounds. Other particularly preferred gap junction channel and/or hemichannel blockers or inhibitor compounds for other connexins are described.

The inventions include pharmaceutical compositions in a form suitable for, or adapted to, treatment of a subject for a cardiovascular disease, disorder or condition. The inventions include pharmaceutical compositions in a form suitable for, or adapted to, treatment of a subject for an ocular disease, disorder or condition, including retinal diseases, disorders and conditions, and other ocular diseases, disorders and conditions characterized in whole or in part by vasucular damage or leak, including for example diseases, disorders and conditions characterized by choroidal neovascularization, damage, leak, edema and/or inflammation.

In one embodiment, the present invention provides connexin gap junction or connexin hemichannel compounds that act as connexin gap junction or connexin hemichannel antagonists, or blockers. In one embodiment, the present invention provides methods for treating angiogenic eye disorders by sequentially administering multiple doses of a connexin gap junction or connexin hemichannel antagonist or blocker to a patient. The methods of the present invention include the administration of multiple doses of a connexin gap junction or connexin hemichannel antagonist or blocker to a patient at a frequency of once every 6, 7, 8 or more weeks.

The methods of the present invention are useful for the treatment of angiogenic eye disorders such as wet age related macular degeneration, dry age related macular degeneration, diabetic retinopathy, diabetic macular edema, central retinal vein occlusion, branch retinal vein occlusion, and corneal neovascularization.

The present invention provides methods that comprise sequentially administering multiple doses of a connexin gap junction or connexin hemichannel antagonist or blocker to a patient over time. In particular, the methods of the invention comprise sequentially administering to the patient a single initial dose of a connexin gap junction or connexin hemichannel antagonist or blocker, followed by one or more secondary doses of the connexin gap junction or connexin hemichannel antagonist or blocker, followed by one or more tertiary doses of the connexin gap junction or connexin hemichannel antagonist or blocker. Thus, according to the methods of the present invention, each secondary dose of connexin gap junction or connexin hemichannel antagonist or blocker is administered 2 to 4 weeks after the immediately preceding dose, and each tertiary dose is administered at least 8 weeks after the immediately preceding dose. An advantage of such a dosing regimen is that, for most of the course of treatment (i.e., the tertiary doses), it allows for less frequent dosing (e.g., once every 8 weeks) compared to prior administration regimens for angiogenic eye disorders which require monthly administrations throughout the entire course of treatment. See, e.g., prescribing information for Lucentis® [ranibizumab], Genentech, Inc.

The expression “angiogenic eye disorder,” as used herein, means any disease of the eye which is caused by or associated with the growth or proliferation of blood vessels or by blood vessel leakage. Non-limiting examples of angiogenic eye disorders that are treatable using the methods of the present invention include age-related macular degeneration (e.g., wet AMD, exudative AMD, etc.), dry AMD, retinal vein occlusion (RVO), central retinal vein occlusion (CRVO; e.g., macular edema following CRVO), branch retinal vein occlusion (BRVO), diabetic macular edema (DME), choroidal neovascularization (CNV; e.g., myopic CNV), iris neovascularization, neovascular glaucoma, post-surgical fibrosis in glaucoma, proliferative vitreoretinopathy (PVR), optic disc neovascularization, corneal neovascularization, retinal neovascularization, vitreal neovascularization, pannus, pterygium, vascular retinopathy, and diabetic retinopathies.

In one embodiment, the disease, disorder or condition is associated with inflammation, e.g., vascular or microvascular inflammation. In another embodiment, the disease, disorder or condition is associated with ischemia and/or oxidative stress, e.g., vascular or microvascular ischemia and/or oxidative stress. In another embodiment, the disease, disorder or condition is associated with edema, e.g., edema associated with vascular or microvascular damage or leak.

In one embodiment, the disease, disorder or condition is associated with a neoplasm, i.e., an abnormal mass of tissue that results when cells divide more than they should or do not die when they should. As used herein, neoplasms are also referred to as tumors and may be benign (not cancer) or malignant (cancer).

In certain embodiments, the cardiovascular disease, disorder or condition is an acute coronary syndrome. The acute coronary syndrome may, for example, be selected from the group consisting of ST-segment elevation myocardial infarction, non-ST-segment elevation myocardial infarction and unstable angina. In other embodiments, the cardiovascular disease, disorder or condition is ischemic heart disease. In other embodiments, the cardiovascular disease, disorder or condition is heart failure (any form). For example, the heart failure may be systolic or diastolic heart failure. The heart failure may result from left ventricular systolic dysfunction. The heart failure may also be a result of right ventricular infarction, pulmonary hypertension, chronic severe tricuspid regurgitation, or arrhythmogenic right ventricular dysplasia. The heart failure may also be a result of diastolic LV dysfunction. In another embodiment the cardioiovascular disease, disorder or condition is ischemic heart disease. Connexin40, 43 and 45 gap junction channel and/or hemichannel blockers or inhibitor compounds are particularly useful for treatment of a cardiovascular disease, disorder or condition.

In one aspect, the invention includes pharmaceutical compositions useful for preventing and/or treating a subject for one or more of the disease, disorder or condition described or referenced herein a subject, including parenteral delivery forms and formulations, as well as other forms of delivery including forms for delivery by infusion, injection and instillation, and delayed, slow, extended or controlled release compositions, devices and matrices, comprising or consisting essentially of therapeutically effective amounts of a gap junction channel and/or hemichannel blocker or inhibitor compound alone or in combination with another cardiovascular therapeutic agent(s), and a pharmaceutically acceptable carrier. In certain preferred embodiments, the pharmaceutical compositions are formulated for intravenous administration, including by infusion or as a bolus. Other formulations for other routes of administration are also within the scope of the invention, including, for example, formulations for nasal, pulmonary, buccal, rectal, transdermal and oral delivery.

In another aspect, the compositions of the invention comprise about 0.01 to about 100 milligrams, about 100 to about 500 milligrams, or about 500 to about 1000 milligrams or more of a compound of the invention, for example, a gap junction channel and/or hemichannel blocker or inhibitor compound or analog, including one or more of SEQ.ID.NOS:1-52 and peptides according to any of Formulae I to II. Other doses are described herein and include doses ranging from at least about 100 nanograms, including, for example at least about 200 micrograms or milligrams, 600 micrograms or milligrams, 2000 micrograms or milligrams, 6000 micrograms or milligrams and at least about 10,000 micrograms or milligrams. Dose concentrations include concentrations of at least about 0.1 moles per liter, including, for example, at least about 0.3, 1.0, 3.0 and 10.0 Moles/L. Dose concentrations also include concentrations of 0.1 micromoles or millimoles/L, 0.3 micromoles or millimoles/L, 1.0 micromoles or millimoles/L, 3.0 micromoles or millimoles/L and 10.0 micromoles or millimoles/L. Dose concentrations may be equivalent to 0.1, 0.3, 1, 3, and 10 to 100 mg/mL and administrable weight doses of 1-10, 10-20, 20-50, 50-100, 100-500 and 500-1000 milligrams/kg (mg/kg). Also within the invention are other doses ranging from 0.1 to 5.0 μg/kg and 0.1 to 10.0 g/kg. These compositions and amounts may be provided as single or muliple doses.

The gap junction channel and/or hemichannel blocker or inhibitor compound (or pharmaceutical formulation comprising the gap junction channel and/or hemichannel blocker or inhibitor compound) may be administered to the patient by any known delivery system and/or administration method. In certain embodiments, the gap junction channel and/or hemichannel blocker or inhibitor compound is administered to the patient by ocular, intraocular, intravitreal or subconjunctival injection. In other embodiments, the gap junction channel and/or hemichannel blocker or inhibitor compound can be administered to the patient by topical administration, e.g., via eye drops or other liquid, gel, ointment or fluid which contains the gap junction channel and/or hemichannel blocker or inhibitor compound and can be applied directly to the eye. Other possible routes of administration include, e.g., intradermal, intramuscular, intraperitoneal, intravenous, subcutaneous, intranasal, epidural, and oral.

The inventions also include methods of treatment of a subject having or at risk for developing a disease, disorder or condition as referenced herein, comprising administering to the subject a therapeutically effective amount of one or more of the compounds or pharmaceutical compositions described herein. In one non-limiting embodiment, the disease, disorder or condition is associated with vessel damage, vessel leak, edema, inflammation, ischemia and/or oxidative stress. In one embodiment, the disease, disorder or condition is an acute coronary syndrome, e.g., ST-segment elevation myocardial infarction, non-ST-segment elevation myocardial infarction or unstable angina. In another embodiment, the cardiovascular disease, disorder or condition is heart failure. In other embodiments, the cardiovascular disease, disorder or condition is ischemic heart disease. In another embodiment, the cardiovascular disease, disorder or condition is stable angina.

The inventions include methods of treating a subject having or at risk for developing, or at risk for progression of, a disease, disorder or condition described or referenced herein, comprising a therapeutically effective amount of a gap junction channel and/or hemichannel blocker or inhibitor compound and a pharmaceutically acceptable carrier. In one embodiment, the gap junction channel and/or hemichannel blocker or inhibitor compound in the pharmaceutical composition comprises or consists essentially of a sequence selected from SEQ.ID.NOS:1-52. In another embodiment, the gap junction channel and/or hemichannel blocker or inhibitor compound in the pharmaceutical composition comprises or consists essentially of a sequence selected from Formula I or Formula II. Gap junction channel and/or hemichannel blocker or inhibitor compounds also include active analogs, variants, truncations, and modified forms of the gap junction channel and/or hemichannel blocker or inhibitor compounds described herein.

In another aspect, the inventions include methods of treating and/or preventing a cardiovascular or ocular disease, disorder or condition that is associated with inflammation, ischemia and/or oxidative stress in a subject by decreasing gap junction channel and/or hemichannel activity in the subject. This may be accomplished, for example, by administering to the subject a composition comprising a therapeutically effective amount of a gap junction channel and/or hemichannel blocker or inhibitor compound, e.g., a gap junction channel and/or hemichannel blocker or inhibitor compound comprising or consisting essentially of a sequence selected from SEQ.ID.NOS:1-52, or a peptide comprising or consisting essentially of a peptide according to any of Formulae I to II, or an analog, variant, truncation or modification thereof, as described for example. In certain embodiments, about 0.01 to about 100, 500 or 1000 nanograms or milligrams or more (e.g., at least about 100 nanograms or milligrams, at least about 500 nanograms or milligrams, or at least about 1000 nanograms or milligrams) of a gap junction channel and/or hemichannel blocker or inhibitor compound or analog is administered per day in single or divided doses or by continuous or periodic release, for example.

In one embodiment, the gap junction channel and/or hemichannel blocker or inhibitor compound is administered in a single dose. In another embodiment, the gap junction channel and/or hemichannel blocker or inhibitor compound is administered in more than one dose. In yet another embodiment, the gap junction channel and/or hemichannel blocker or inhibitor compound is administered or released continuously over a period of time, for example a predetermined period of time. In still another embodiment, a cardiovascular treatment agent or an ocular medicine (e.g., a VEGF antagonist) is administered or co-administered with the gap junction channel and/or hemichannel blocker or inhibitor compound. Such other cardiovascular treatment agents include nitrates, β-blockers, calcium channel blockers (particularly for stable or unstable angina, but also for heart failure in the case of β-blockers), diuretic agents, vasodilator agents, positive inotropes, ACE inhibitors and aldosterone antagonists, e.g. spironolactone (particularly for heart failure), blood thinning therapeutics (e.g., aspirin, heparins, warfarins) and nitroglycerin (particularly for MI).

The inventions also provide a method for performing angioplasty on a patient in need thereof, comprising administering a gap junction channel and/or hemichannel blocker or inhibitor compound to the patient during the angioplasty procedure. In a further embodiment, the method comprises or further comprises administering a gap junction channel and/or hemichannel blocker or inhibitor compound to the patient prior to the angioplasty procedure. In a further embodiment, the method comprises or further comprises administering a gap junction channel and/or hemichannel blocker or inhibitor compound to the patient following the angioplasty procedure. In other embodiments, a gap junction channel and/or hemichannel blocker or inhibitor compound is administerd to the patient before, during, and/or after the angioplasty procedure, in any combination.

Also provided are methods for increasing the time during which thrombolytic therapy will be effective following the first symptom of cardiac distress, comprising administering a therapeutically effective amount of a gap junction channel and/or hemichannel blocker or inhibitor compound after the onset of one or more of the following symptoms: chest pain lasting longer than 15 minutes, chest pain at rest, chest pain following minimal exertion, nausea, shortness of breath, palpitations, or dizziness.

In another aspect, the treated subject is a mammal, preferably a human. Other mammals include domestic and farm animals, and zoo, sports, or pet animals, such as dogs, horses, and cats.

The inventions also include articles of manufacture comprising package material containing one or more of the compounds or pharmaceutical compositions described herein. Then inventions also include articles of manufacture comprising package material containing one or more of the compounds or pharmaceutical compositions described herein, together with instructions for use in or on a subject in order to prevent and/or treat a disease, disorder or condition as noted herein. In one embodiment, a vascular disease, disorder or condition is referred to in the instructions that is associated with inflammation, angiogeneisis and/or vessel leak, ischemia and/or oxidative stress. In another embodiment the disease, disorder or condition is referred to in the instructions is a cardioiovascular disease, disorder or condition. In one embodiment, the disease, disorder or condition referred to in the instructions is an angiogenic eye disorder. The instructions may be electronic and/or associated with a website.

The inventions also include methods of preparing a medicament for preventing or treating one or more of the diseases, disorders or conditions referenced herein, comprising bringing together a therapeutically effective amount of a compound referenced herein, e.g., a gap junction channel and/or hemichannel blocker or inhibitor compound or analog or variant, and a pharmaceutically acceptable carrier. In one embodiment the gap junction channel and/or hemichannel blocker or inhibitor compound comprises a sequence selected from SEQ.ID.NOS:1 to 52. In another embodiment the gap junction channel and/or hemichannel blocker or inhibitor compound is a compound selected from one or more of Formulae I-II. In one embodiment the medicament is formulated for parenteral administration. In one embodiment the medicament is formulated for ocular administration. In another embodiment the medicament is formulated for topical administration. In another embodiment the medicament is formulated for oral administration.

Treatment of a subject as provided herein with one or more compounds or pharmaceutical compositions as described herein may comprise their simultaneous, separate, sequential or sustained administration.

Pharmaceutical compositions useful for preventing and/or treating a disease, disorder or conditioned referenced or described herein are also provided in the form of a combined preparation, for example, as an admixture of two or more gap junction channel and/or hemichannel blocker or inhibitor compounds, analogs or variants.

The term “a combined preparation” includes not only physical combinations of compounds, but compounds provided as a “kit of parts” in the sense that the combination partners as defined above can be dosed independently or by use of different fixed combinations with distinguished amounts of the combination partners (a) and (b), i.e. simultaneously, separately or sequentially. The parts of the kit can then, for example, be administered simultaneously or chronologically staggered, that is at different time points and with equal or different time intervals for any part of the kit of parts.

In another aspect, the invention includes methods for administering a therapeutically effective amount of a gap junction channel and/or hemichannel blocker or inhibitor compound or analog or variant, alone or in combination with another therapeutic agent, formulated in a delayed release preparation, a slow release preparation, an extended release preparation, a controlled release preparation, and/or in a repeat action preparation to a subject having or at risk for developing a disease, disorder or condition described or referenced herein.

In certain other aspects, the invention also relates to methods of using such compositions to treat subjects suffering from or at risk for said diseases, disorders and conditions.

In other aspects, the inventions include methods and compositions for preventing and/or treating a subject having or suspected of having or predisposed to, or at risk for, any diseases, disorders and/or conditions characterized in whole or in part by vascular damage or leak.

According to one aspect, the present invention is directed to methods of halting or decreasing or providing relief from the symptoms of a cardiovascular or ocular disorder.

The invention includes an article of manufacture comprising packaging material containing one or more dosage forms as described herein, wherein the packaging material has a label that indicates that the dosage form can be used for a subject having or suspected of having or predisposed to any of the diseases, disorders and/or conditions described or referenced herein.

The invention includes methods for the use of a therapeutically effective amount of a gap junction channel and/or hemichannel blocker or inhibitor compound(s) in the manufacture of a dosage form useful for preventing and/or treating a cardiovascular or ocular disorder. Such dosage forms include, for example, oral delivery forms and formulations, well as other forms of delivery including forms for delivery by infusion, injection and instillation, and compositions and devices including slow-release, extended release, and delayed release compositions, depots and matrices, for example. Such dosage forms include those for the treatment of a subject as disclosed herein.

These and other aspects of the present inventions, which are not limited to or by the information in this Brief Summary, are provided below.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1A shows (FIG. 1A) an immunolabel image of Connexin43 (red) in untreated control with DAPI-labelled nuclei (blue) (upper panel). The distribution of connexin43 following 2- (middle panel) or 6-hour (lower panel) exposure to VDCFLSRPTEKT (Peptide5; 500 μM) (SEQ ID NO: 45). White arrows highlight patterns of cytoplasmic streaming that extend from the membrane towards the center of the cell. Fragmented Connexin43 plaques along the cell-to-cell interface following 6-hour exposure to Peptide5. Scale bars=30 μm. FIG. 1B shows quantification of the total area of Connexin43 plaques per cell represented as a percentage of untreated control following 2- or 6-hour treatment with Peptide5 or LaCl₃ or CBX (500 μM). A significant reduction in connexin43 labeling per cell compared to untreated control was evident following 2- and 6-hour treatment with CBX. Values represent mean±S.E.M (One-way ANOVA, Dunnett's post-hoc test). FIG. 1C shows no significant difference in connexin43 labelling was present between untreated control and 6 hour treatment with Peptide5 (50-500 μM) **p=0.0043,***p<0.0001. Bars represent mean±S.E.M (n=3). One-way ANOVA Tukey's multiple-comparisons test. Peptide5 alters the distribution of connexin43 gap junction plaques in ARPE-19 cells.

FIGS. 2A and 2B shows data and microscopy images demonstrating that VDCFLSRPTEKT (Peptide5) (SEQ ID NO: 45) analogs with single alanine substitutions are not significantly different to the native Peptide5 in a scrape loading assay of hCMVEC cells in vitro. FIG. 2A shows positive LY dye transfer between coupled gap junction channels is represented as a percentage of the untreated control. LY dye transfer was significantly reduced compared to the untreated control following 2-hour exposure to CBX. FIG. 2B shows native Peptide5 and its analogues with single alanine substitutions (Ala-1 to Ala-12) also significantly attenuated LY dye transfer compared against untreated control. These analogs were not significantly different to the native Peptide5, and only the CBX control attenuated LY dye transfer significantly more than the native Peptide5. Concentration used for all treatment groups was 100 μM. **p<0.005,****p<0.0001. All values represent mean±standard error of the mean. One-way ANOVA, Dunnett's multiple comparison test against control or native Peptide5.

FIG. 3. Shows a bar chart demonstrating that single substitutions of alanine in VDCFLSRPTEKT (Peptide5) (SEQ ID NO: 45) affect the function of the peptide to protect against ischemic injury-induced ATP release from hCMVEC cells in vitro. Quantification of the total extracellular ATP release is presented as a percentage of the injury control for each treatment group. Treatment with CBX, LaC13 and native Peptide5 (all at 100 μM) significantly reduced ATP release, ****p<0.0001 against injury control. Ala-2, Ala-3, Ala-5, Ala-7, Ala-8 and Ala-11 (all at 100 μM), were significantly different to native Peptide5 (100 μM) but not against injury indicating loss of function with these peptides containing alanine substitutions at sites VDCFLSRPTEKT (SEQ ID NO: 45). *p<0.03, **p<0.01, ***p=0.0002. When compared against native Peptide5, no significant differences were observed with Ala-1, Ala-4, Ala-6, Ala-9, Ala-10, Ala-12. All values represent mean±standard error of the mean. One-way ANOVA, Dunnett's comparison test against Injury or native Peptide5.

FIG. 4. Shows a bar chart demonstrating that truncated sequences of VDCFLSRPTEKT (Peptide5) (SEQ ID NO: 45) show altered efficacy against ischaemic injury induced ATP release from hCMVEC cells in vitro. Quantification of the total extracellular ATP release is presented as a percentage of the injury control for each treatment group. Treatment with native Peptide5 (100 μM) and Mod-1 (SEQ ID NO: 85) significantly reduced ATP release, ****p<0.0001 against injury control. In contrast, significantly greater ATP release was observed following treatment with Mod-2 (SEQ ID NO: 86), Mod-3 (SEQ ID NO: 77), Mod-4 (SEQ ID NO: 87), Mod-5 (SEQ ID NO: 88) and Mod-6 (SEQ ID NO: 89) (all at 100 μM) compared to native Peptide5 (100 μM), which indicates loss of function with these modifications. *p=0.0231 and ****p<0.0001 compared to the native peptide. n=8 wells, 4 independent experiments. One-way ANOVA, Dunnett's multiple comparison test against Injury or native Peptide5.

FIG. 5A shows microscopy images showing the effect of VDCFLSRPTEKT (Peptide5) (SEQ ID NO: 45) and SRPTEKT motif (SEQ ID NO: 77) on Lucifer yellow dye transfer in a scrape loading assay of hCMVEC cells in vitro. Positive LY transfer in the control that is reduced in Peptide5 and SRPTEKT (SEQ ID NO: 77). FIG. 5B shows a bar chart showing positive LY dye transfer between coupled gap junction channels is represented as a percentage of the untreated control. LY dye transfer was significantly reduced compared to the untreated control following 2-hour exposure to CBX. SRPTEKT (SEQ ID NO: 77) significantly reduced LY dye transfer that was comparable to the native Peptide5. Concentration used for all treatment groups was 100 μM. **p=0.0057 (Pep5), **p=0.0028 ****p<0.0001. There was no significant difference with scrambled peptide. All values represent mean±SEM. One-way ANOVA, Dunnett's comparison test against Injury or native Peptide5.

FIGS. 6A and 6B shows a figure and data summarizing a competition assay with synthetic peptides derived from the extracellular loops of connexin43 and VDCFLSRPTEKT (Peptide5) (SEQ ID NO: 45). FIG. 6A show synthetic extracellular loops derived from Connexin43. FIG. 6B shows total extracellular ATP released from hCMVEC cells in response to simulated ischaemic injury is represented as a percentage of the injury control for each treatment group. A significant increase in the total ATP released from hCMVEC cells was present following 2 hours of simulated ischaemic injury. ATP release significantly increased in equimolar concentration of native Peptide5 and EL2c indicating loss of Peptide5 function. CBX, Peptide5 and extracellular loops were used at a concentration of 100 μM. ****p<0.0001. There was a significant difference in the total ATP release between injury and Peptide5 A significant reduction in the total ATP release was present with 100 μM native Peptide5. All values represent mean±standard error of the mean. One-way ANOVA, Dunnett's comparison test against Injury or native Peptide5.

### DETAILED DESCRIPTION

Practice of the present inventions may include or employ various conventional techniques of molecular biology (including recombinant techniques), microbiology, cell biology, biochemistry, nucleic acid chemistry, and immunology, which are within the skill of the art. Such techniques are explained fully in the literature, and include but are not limited to, by way of example only, Molecular Cloning: A Laboratory Manual, second edition (Sambrook et al., 1989) and Molecular Cloning: A Laboratory Manual, third edition (Sambrook and Russel, 2001), jointly and individually referred to herein as “Sambrook”; Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987, including supplements through 2001); PCR: The Polymerase Chain Reaction, (Mullis et al., eds., 1994); Current Protocols in Immunology (J. E. Coligan et al., eds., 1991); The Immunoassay Handbook (D. Wild, ed., Stockton Press NY, 1994); Bioconjugate Techniques (Greg T. Hermanson, ed., Academic Press, 1996); Methods of Immunological Analysis (R. Masseyeff, W. H. Albert, and N. A. Staines, eds., Weinheim: VCH Verlags gesellschaft mbH, 1993), Harlow and Lane (1988) Antibodies, A Laboratory Manual, Cold Spring Harbor Publications, New York, and Harlow and Lane (1999) Using Antibodies: A Laboratory Manual Cold Spring Harbor Laboratory Press, Cold Spring Harbor, N.Y. (jointly and individually referred to herein as Harlow and Lane), Beaucage et al. eds., Current Protocols in Nucleic Acid Chemistry John Wiley & Sons, Inc., New York, (2000); and Agrawal, ed., Protocols for Oligonucleotides and Analogs, Synthesis and Properties Humana Press Inc., New Jersey, (1993).

It is to be understood that the inventions are not limited to the particular methodology, protocols, constructs, and reagents described herein and as such may vary. It is also to be understood that the terminology used herein is for the purpose of describing particular embodiments only, and is not intended to limit the scope of the present invention, which will be limited only by the appended claims. As used herein and in the appended claims, the singular forms “a,” “an,” and “the” include plural reference unless the context clearly indicates otherwise. Thus, for example, reference to a “ap junction channel and/or hemichannel blocker or inhibitor compounds” is a reference to one or more such peptides and includes equivalents thereof now known or later developed. Unless defined otherwise, all technical and scientific terms used herein have the same meaning as commonly understood to one of ordinary skill in the art to which the inventions belong. Although any methods, devices, and materials similar or equivalent to those described herein can be used in the practice or testing of the invention, the preferred methods, devices and materials are now described. It is intended that reference to a range of numbers disclosed herein (for example 1 to 12) also incorporates reference to all related numbers within that range (for example, 1, 1.1, 2, 3, 3.9, 4, 5, 6, 6.5, 7, 8, 9.5, 10, 11 and 12) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner. The following terms have the following meanings when used herein.

The inventions described and claimed herein relate to connexin gap junctions and connexin hemichannels, including connexin25, connexin26, connexin29 (connexin30.2), connexin30, connexin30.3, connexin31, connexin31.1, connexin31.3, connexin31.9, connexin32, connexin36, connexin37, connexin40, connexin40.1, connexin43, connexin45 connexin46, connexin46.6 (connexin47), connexin50, connexin58, connexin59, connexin62, and others.

In one embodiment, the peptides and peptide mimetics described herein are gap junction channel inhibitors. In one embodiment, the gap junction comprises a connexin43 protein. In one embodiment, the gap junction comprises a connexin45 protein. In another embodiment, the peptides and peptide mimetics described herein are inhibitors of gap junction hemichannel opening. In one embodiment, the gap junction hemichannel comprises a connexin43 protein. In one embodiment, the gap junction hemichannel comprises a connexin45 protein. In other embodiments, the gap junctions and hemichannels comprise connexin25, connexin26, connexin29 (connexin30.2), connexin30, connexin30.3, connexin31, connexin31.1, connexin31.3, connexin31.9, connexin32, connexin36, connexin37, connexin40, connexin40.1, connexin46, connexin46.6 (connexin47), connexin50, connexin58, connexin59, connexin62 proteins, and the peptides and peptide mimetics described herein are inhibitors of one or more of these gap junction channels and corresponding hemichannels.

Using a various connexin mimetic peptides, as shown in the Examples, peptide sequence analogs of the extracellular loops of the human connexin proteins have been shown to prevent endothelial cell loss and reduces vascular permeability in preclinical models, including human microvascular endothelial cells, where it was demonstrated that peptide compounds of the invention can inhibit hemichannel-mediated ATP release from endothelial cells. Peptide sequence specificity and sensitivity to inhibition of hemichannel-mediated ATP release and the uncoupling of gap junctions was also demonstrated. The SRPTEKT motif (SEQ ID NO: 77) is central to function but on its own is not sufficient to inhibit hemichannels at the concentrations used. However, working at high concentrations, SRPTEKT motif (SEQ ID NO: 77) alone reduced gap junction communication to an extent that was comparable to VDCFLSRPTEKT (SEQ ID NO: 45). An understanding of the dose dependent mode of action of the peptides also supports their development as treatment for diseases, disorders and conditions, including, for example, retinal injury and disease, cardiovascular disease, and neoplastic cancers.

Amino acids used in compounds provided herein (e.g. peptides and proteins) can be genetically encoded amino acids, naturally occurring non-genetically encoded amino acids, or synthetic amino acids. Both L-and D-enantiomers of any of the above can be utilized in the compounds. The following abbreviations may be used herein for the following genetically encoded amino acids (and residues thereof): alanine (Ala, A); arginine (Arg, R); asparagine (Asn, N); aspartic acid (Asp, D); cyteine (Cys, C); glycine (Gly, G); glutamic acid (Glu, E); glutamine (Gln, Q); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V).

Certain commonly encountered amino acids that are not genetically encoded and that can be present in active compounds of the invention include, but are not limited to, β-alanine (b-Ala) and other omega-amino acids such as 3-aminopropionic acid (Dap), 2,3-diaminopropionic acid (Dpr, Z), 4-aminobutyric acid and so forth; α-aminoisobutyric acid (Aib); ε-aminohexanoic acid (Aha); δ-aminovaleric acid (Ava); methylglycine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle, J); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); beta.-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH2)); N-methyl valine (MeVal); homocysteine (hCys); 3-benzothiazol-2-yl-alanine (BztAla, B); and homoserine (hSer). Additional amino acid analogs contemplated include phosphoserine, phosphothreonine, phosphotyrosine, hydroxyproline, gamma-carboxyglutamate, hippuric acid, octahydroindole-2-carboxylic acid, statine, α-methyl-alanine, para-benzoyl-phenylalanine, propargylglycine, and sarcosine. Peptides that are encompassed within the scope of the invention can have any of the foregoing amino acids in the L- or D-configuration, or any other amino acid described herein or known in the art, whether currently or in the future, whilst retaining a biological activity.

Amino acids that are substitutable for each other generally reside within similar classes or subclasses. As known to one of skill in the art, amino acids can be placed into different classes depending primarily upon the chemical and physical properties of the amino acid side chain. For example, some amino acids are generally considered to be hydrophilic or polar amino acids and others are considered to be hydrophobic or nonpolar amino acids. Polar amino acids include amino acids having acidic, basic or hydrophilic side chains and nonpolar amino acids include amino acids having aromatic or hydrophobic side chains. Nonpolar amino acids may be further subdivided to include, among others, aliphatic amino acids. The definitions of the classes of amino acids as used herein are as follows:

“Nonpolar Amino Acid” refers to an amino acid having a side chain that is uncharged at physiological pH, that is not polar and that is generally repelled by aqueous solution. Examples of genetically encoded hydrophobic amino acids include Ala, Ile, Leu, Met, Trp, Tyr and Val. Examples of non-genetically encoded nonpolar amino acids include t-BuA, Cha and Nle.

“Aromatic Amino Acid” refers to a nonpolar amino acid having a side chain containing at least one ring having a conjugated π-electron system (aromatic group). The aromatic group may be further substituted with substituent groups such as alkyl, alkenyl, alkynyl, hydroxyl, sulfonyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, β-2-thienylalanine, 3-benzothiazol-2-yl-alanine, 1,2,3,4-tetrahydroisoquinoline-3-carboxylic acid, 4-chlorophenylalanine, 2-fluorophenylalanine, 3-fluorophenylalanine and 4-fluorophenylalanine.

“Aliphatic Amino Acid” refers to a nonpolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Polar Amino Acid” refers to a hydrophilic amino acid having a side chain that is charged or uncharged at physiological pH and that has a bond in which the pair of electrons shared in common by two atoms is held more closely by one of the atoms. Polar amino acids are generally hydrophilic, meaning that they have an amino acid having a side chain that is attracted by aqueous solution. Examples of genetically encoded polar amino acids include asparagine, cysteine, glutamine, lysine and serine. Examples of non-genetically encoded polar amino acids include citrulline, homocysteine, N-acetyl lysine and methionine sulfoxide.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include ornithine, 2,3-diaminopropionic acid, 2,4-diaminobutyric acid and homoarginine.

“Ionizable Amino Acid” refers to an amino acid that can be charged at a physiological pH. Such ionizable amino acids include acidic and basic amino acids, for example, D-aspartic acid, D-glutamic acid, D-histidine, D-arginine, D-lysine, D-hydroxylysine, D-ornithine, L-aspartic acid, L-glutamic acid, L-histidine, L-arginine, L-lysine, L-hydroxylysine or L-ornithine.

As will be appreciated by those having skill in the art, the above classifications are not absolute. Several amino acids exhibit more than one characteristic property, and can therefore be included in more than one category. For example, tyrosine has both a nonpolar aromatic ring and a polar hydroxyl group. Thus, tyrosine has several characteristics that could be described as nonpolar, aromatic and polar. However, the nonpolar ring is dominant and so tyrosine is generally considered to be nonpolar. Similarly, in addition to being able to form disulfide linkages, cysteine also has nonpolar character. Thus, while not strictly classified as a hydrophobic or nonpolar amino acid, in many instances cysteine can be used to confer hydrophobicity or nonpolarity to a peptide.

In some embodiments, polar amino acids contemplated by the present invention include, for example, arginine, asparagine, aspartic acid, cysteine, glutamic acid, glutamine, histidine, homocysteine, lysine, hydroxylysine, ornithine, serine, threonine, and structurally related amino acids. In one embodiment the polar amino is an ionizable amino acid such as arginine, aspartic acid, glutamic acid, histidine, hydroxylysine, lysine, or ornithine.

Examples of polar or nonpolar amino acid residues that can be utilized include, for example, alanine, valine, leucine, methionine, isoleucine, phenylalanine, tryptophan, tyrosine and the like.

As used herein, a “cardiovascular disorder” is any cardiovascular disease, disorder or condition that involves or may be characterized at least in part by oxidative stress and/or ischemia.

During physiological processes molecules undergo chemical changes involving reducing and oxidizing reactions. A molecule with an unpaired electron can combine with a molecule capable of donating an electron. The donation of an electron is termed as oxidation whereas the gaining of an electron is called reduction. Reduction and oxidation can render the reduced molecule unstable and make it free to react with other molecules to cause damage to cellular and sub-cellular components such as membranes, proteins and DNA. As used herein, “oxidative stress” refers in one example to excessive production of reactive oxidant species (ROS) resulting in oxidative stress/nitrosative stress, a process that is an important mediator of cell damage. Important aspects of redox imbalance that triggers the activity of a number of signaling pathways including transcription factors activity, a process that is ubiquitous in cardiovascular disease related to ischemia/reperfusion injury, for example. Reactive oxidant species can originate from a variety of sources such as nitric oxide (NO) synthase (NOS), xanthine oxidases (XO), the cyclooxygenases, nicotinamide adenine dinucleotide phosphate (NAD(P)H) oxidase isoforms and metal-catalyzed reactions. These include free radicals such as superoxide anion (O₂⁻), hydroxyl radical (HO.), lipid radicals (ROO⁻) and nitric oxide (NO). Other reactive oxygen species, for example, hydrogen peroxide (H₂O₂), peroxynitrite (ONOO⁻) and hypochlorous acid (HOCl), although are not free radicals but have oxidizing effects that contribute to oxidative stress.

“Ischemia” is a condition that occurs when blood flow and oxygen are diminished in a particular part of the body. Cardiac or retinal ischemia is the name for this condition when the heart or retina is the body part targeted, for example.

Ocular disorders include, for example, angiogenic eye disorders, e.g., any disease, disorder or condition of the eye which is caused by or associated with the growth or proliferation of blood vessels or by blood vessel leakage. Non-limiting examples that are treatable using the methods of the present invention include age-related macular degeneration (e.g., wet AMD, exudative AMD, etc.), dry AMD, retinal vein occlusion (RVO), central retinal vein occlusion (CRVO; e.g., macular edema following CRVO), branch retinal vein occlusion (BRVO), diabetic macular edema (DME), choroidal neovascularization (CNV; e.g., myopic CNV), iris neovascularization, neovascular glaucoma, post-surgical fibrosis in glaucoma, proliferative vitreoretinopathy (PVR), optic disc neovascularization, corneal neovascularization, retinal neovascularization, vitreal neovascularization, pannus, pterygium, vascular retinopathy, and diabetic retinopathies.

The art is familiar with modification of peptides, for example, by polymer conjugation or glycosylation. The terms “gap junction channel blocker or inhibitor compound” and “hemichannel blocker or inhibitor compound” includes modified peptides including peptides conjugated to a polymer such as PEG, and may be comprised of one or more additional derivitizations of cysteine, lysine, or other residues. In addition, the hemichannel peptide agent agent may comprise a linker or polymer, wherein the amino acid to which the linker or polymer is conjugated may be a non-natural amino acid according to the present invention, or may be conjugated to a naturally encoded amino acid utilizing techniques known in the art such as coupling to lysine or cysteine.

Substitutions, deletions, modifications or additions of amino acids described herein in reference to compounds of the invention, for example, SEQ ID NO: 1-52, or other peptides as defined, for example, in Formula I and II, are intended to also refer to substitutions, deletions, modifications or additions in corresponding positions in fusions, variants, fragments, conjugations, etc.

The terms “gap junction channel blocker or inhibitor compound” and “hemichannel blocker or inhibitor compound” also encompass homodimers, heterodimers, homomultimers, and heteromultimers that are linked, including but not limited to those linked directly via non-naturally encoded amino acid side chains, either to the same or different non-naturally encoded amino acid side chains, to naturally-encoded amino acid side chains, or indirectly via a linker. Exemplary linkers include small organic compounds, water soluble polymers of a variety of lengths such as poly(ethylene glycol) or polydextran or polypeptides of various lengths.

The term “linker” is used herein to refer to groups or bonds that normally are formed as the result of a chemical reaction and typically are covalent linkages. Hydrolytically stable linkages means that the linkages are substantially stable in water and do not react with water at useful Ph values, including but not limited to, under physiological conditions for an extended period of time, perhaps even indefinitely. Hydrolytically unstable or degradable linkages mean that the linkages are degradable in water or in aqueous solutions, including for example, blood. Enzymatically unstable or degradable linkages mean that the linkage can be degraded by one or more enzymes. As understood in the art, PEG and related polymers may include degradable linkages in the polymer backbone or in the linker group between the polymer backbone and one or more of the terminal functional groups of the polymer molecule. For example, ester linkages formed by the reaction of PEG carboxylic acids or activated PEG carboxylic acids with alcohol groups on a biologically active agent generally hydrolyze under physiological conditions to release the agent. Other hydrolytically degradable linkages include, but are not limited to, carbonate linkages; imine linkages resulted from reaction of an amine and an aldehyde; phosphate ester linkages formed by reacting an alcohol with a phosphate group; hydrozone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

The term “active” or “biologically active” when used herein means any substance which can affect any physical or biochemical properties of a biological system, pathway, molecule, or interaction relating to an organism, including but not limited to, viruses, bacteria, bacteriophage, transposons, prions, insects, fungi, plants, animals, and humans. In particular, as used herein, biologically active molecules include, but are not limited to, any substance intended for cure, mitigation, treatment, or prevention of the diseases, disorders and conditions described or referenced herein in humans or other animals.

As used herein, the term “water soluble polymer” refers to any polymer that is soluble in aqueous solvents. Linkage of water soluble polymers to a gap junction channel blocker or inhibitor compound or a hemichannel blocker or inhibitor compound can result in changes including, but not limited to, increased or modulated serum half-life, or increased or modulated therapeutic half-life relative to the unmodified form, modulated immunogenicity, modulated physical association characteristics such as aggregation and multimer formation, altered receptor binding, and altered receptor dimerization or multimerization. The water soluble polymer may or may not have its own biological activity, and may be utilized as a linker for attaching a gap junction channel or hemichannel blocker or inhibitor compound to other substances. Suitable polymers include, but are not limited to, polyethylene glycol, polyethylene glycol propionaldehyde, mono C1-C10 alkoxy or aryloxy derivatives thereof (described in U.S. Pat. No. 5,252,714), monomethoxy-polyethylene glycol, polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polypropylene oxide/ethylene oxide copolymer, polyoxyethylated polyol, heparin, heparin fragments, polysaccharides, oligosaccharides, glycans, cellulose and cellulose derivatives, including but not limited to methylcellulose and carboxymethyl cellulose, starch and starch derivatives, polypeptides, polyalkylene glycol and derivatives thereof, copolymers of polyalkylene glycols and derivatives thereof, polyvinyl ethyl ethers, and alpha-beta-poly[(2-hydroxyethyl)-DL-aspartamide, and the like, or mixtures thereof. Examples of such water soluble polymers include, but are not limited to, polyethylene glycol and serum albumin.

As used herein, the term “polyalkylene glycol” or “poly(alkene glycol)” refers to polyethylene glycol (poly(ethylene glycol)), polypropylene glycol, polybutylene glycol, and derivatives thereof. The term “polyalkylene glycol” and/or “polyethylene glycol” encompasses both linear and branched polymers and average molecular weights of between 0.1 kDa and 100 kDa or more. Other exemplary embodiments are listed, for example, in commercial supplier catalogs, such as Shearwater Corporation's catalog “Polyethylene Glycol and Derivatives for Biomedical Applications” (2001).

As used herein, the term “modified serum half-life” means an increased circulating half-life of a modified gap junction channel or hemichannel blocker or inhibitor compound relative to its non-modified form. Serum half-life is measured by taking blood samples at various time points after administration of a gap junction channel or hemichannel blocker or inhibitor compound and determining the concentration of that molecule in each sample. Correlation of the serum concentration with time allows calculation of the serum half-life. Increased serum half-life desirably has at least about two-fold, but a smaller increase may be useful, for example where it enables a satisfactory dosing regimen or avoids a toxic effect. In some embodiments, the increase is at least about three-fold, at least about five-fold, or at least about ten-fold or more.

The term “modified therapeutic half-life” as used herein means an increase in the half-life of the therapeutically effective amount of a modified a gap junction channel or hemichannel blocker or inhibitor compound relative to its non-modified form. Therapeutic half-life is measured by measuring pharmacokinetic and/or pharmacodynamic properties of the molecule at various time points after administration. Increased therapeutic half-life desirably enables a particular beneficial dosing regimen, a particular beneficial total dose, or avoids an undesired effect. In some embodiments, the increased therapeutic half-life results from increased potency, increased or decreased binding of the modified molecule to its target, increased or decreased breakdown of the molecule by enzymes such as proteases, or an increase or decrease in another parameter or mechanism of action of the non-modified molecule.

The term “isolated,” when applied to a peptide, denotes that the peptide is free of at least some of the cellular or other biological components with which it is associated in the natural state, or that the peptide has been concentrated to a level greater than the concentration of its in vivo or in vitro production. It can be in a homogeneous or substantially homogenous state. Isolated substances can be in either a dry or semi-dry state, or in solution, including but not limited to, an aqueous solution. It can be a component of a pharmaceutical composition that comprises additional pharmaceutically acceptable carriers and/or excipients. Purity and homogeneity are typically determined using analytical chemistry techniques such as polyacrylamide gel electrophoresis or high performance liquid chromatography, for example.

By “substantially pure” is meant a degree of purity of a gap junction channel or hemichannel blocker or inhibitor compound where there is at least 70% of a gap junction channel or hemichannel blocker or inhibitor compound, more preferably at least 80%, and even more preferably increasing to at least 90%, 95% or 99%. A particularly preferred purity is at least 95%. By “essentially pure” is meant that the composition is at least 90% or more pure for the desired gap junction channel or hemichannel blocker or inhibitor compound. A peptide which is the predominant species present in a preparation is also substantially purified.

The term “effective amount” as used herein refers to that amount of a gap junction channel or hemichannel blocker or inhibitor compound being administered that will relieve to some extent one or more of the symptoms of the disease, condition or disorder being treated. Compositions containing the gap junction channel or hemichannel blockers or inhibitor compounds described herein can be administered for prophylactic, enhancing, and/or therapeutic treatments.

As used herein, “subject” refers to any mammal, including humans, domestic and farm animals, and zoo, sports, or pet animals, such as dogs, horses, cats, sheep, pigs, cows, etc. The preferred mammal herein is a human, including adults, children, and the elderly. Preferred sports animals are horses and dogs. Preferred pet animals are dogs and cats.

As used herein, “preventing” means preventing in whole or in part, ameliorating or controlling, reducing, lessening, or decreasing, or retarding or halting.

As used herein, a “therapeutically effective amount” in reference to the compounds or compositions of the instant invention refers to the amount sufficient to induce a desired biological, pharmaceutical, or therapeutic result. That result can be alleviation of one or more of the signs, symptoms, or causes of a disease or disorder or condition, or any other desired alteration of a biological system. In the present invention, by way of example. the result will involve the treatment, prevention and/or reduction of one or more of symptoms of a cardiovascular or ocular disorder, including, for example, an angiogenic, inflammatory or edematous ocular condition, or an acute coronary syndrome, e.g., an ischemic heart disease, and any ocular cardiovascular disorder, disease, or condition, for example, that involves ischemia and/or oxidative stress.

As used herein, the terms “treating” and “treatment” refer to both therapeutic treatment and prophylactic or preventative measures.

“Analogs” or “peptide analogs” refer to the compounds with properties analogous to those of the template peptide and may be non-peptide drugs. “Peptidomimetics” (also known as “mimetic peptides”), which include peptide-based compounds, also include such non-peptide based compounds such as peptide analogs. Peptidomimetics that are structurally similar to therapeutically useful peptides may be used to produce an equivalent or enhanced therapeutic or prophylactic effect. Generally, peptidomimetics are structurally identical or similar to a paradigm polypeptide (i.e., a polypeptide that has a biological or pharmacological function or activity), but can also have one or more peptide linkages optionally replaced by a linkage selected from the group consisting of, for example, —CH2NH—, —CH2S—, —CH2-CH2—, —CH═CH— (cis and trans), —COCH2-, —CH(OH)CH2-, and —CH2SO—. The mimetic can be either entirely composed of natural amino acids, or non-natural analogues of amino acids, or, is a chimeric molecule of partly natural peptide amino acids and partly non-natural analogs of amino acids. The mimetic can also comprise any amount of natural amino acid conservative substitutions as long as such substitutions also do not substantially alter mimetic activity.

In general, the term “peptide” refers to any polymer of two or more individual amino acids (whether or not naturally occurring) linked via peptide bonds, as occur when the carboxyl carbon atom of the carboxylic acid group bonded to the alpha-carbon of one amino acid (or amino acid residue) becomes covalently bound to the amino nitrogen atom of the amino group bonded to the alpha-carbon of an adjacent amino acid. These peptide bond linkages, and the atoms comprising them (i.e., alpha-carbon atoms, carboxyl carbon atoms (and their substituent oxygen atoms), and amino nitrogen atoms (and their substituent hydrogen atoms)) form the “polypeptide backbone” of the protein. In addition, as used herein, the terms “polypeptide” and “peptide” may be used interchangeably. Similarly, protein fragments, analogs, derivatives, and variants are may be referred to herein as “peptides” or “peptide agents.”. The term “fragment” of a peptide refers to a polypeptide comprising fewer than all of the amino acid residues of the peptide.

As used herein, “simultaneously” is used to mean that the one or more agents of the invention are administered concurrently, whereas the term “in combination” is used to mean they are administered, if not simultaneously or in physical combination, then “sequentially” within a timeframe that they both are available to act therapeutically. Thus, administration “sequentially” may permit one agent to be administered within minutes (for example, 1, 2, 3, 4, 5, 10, 15, 20, 25, 30) minutes or a matter of hours, days, weeks or months after the other provided that both the gap junction channel or hemichannel blocker or inhibitor compound and another ocular or cardiovascular therapeutic agent, for example, are concurrently present in effective amounts. The time delay between administration or administrations of the components will vary depending on the exact nature of the components, the interaction therebetween, and their respective half-lives.

### Hemichannel Peptide Agents

Gap junction channel and hemichannel blockers or inhibitor compounds of the invention described herein are capable of modulating one or more of the symtoms of a microvascular disorder. Preferably, the microvascular disorder is angiogenic eye disorder or ocular injury or cardiovascular disorder, but others are intended as described herein, including those characterized by inflammation, edema, and/or vessel damage or leak.

The present invention provides for the production of peptides and peptide mimetics having pharmacokinetic and pharmacodynamic properties useful for modulating gap junction communication and hemichannel opening and other activities described herein, and their use in the treatment of diseases, disorders and conditions where modulation of gap junction activity and/or hemichannel opening and hemichannel activity is therapeutically beneficial.

The inventions described and claimed herein relate to peptides and peptidomimetics that bind to gap junction and gap junction hemichannel proteins. Applicants' inventions also relate to pharmaceuticals, compositions and methods useful for treating, preventing and ameliorating the effects of vascular diseases, disorders and conditions, as well as articles and kits comprising such compounds and compositions. In one aspect, the inventions relate to compounds for controlling gap junction protein channel activity. In another aspect, the inventions relate to compounds to control gap junction hemichannel activity.

In one embodiment, the peptides and peptide mimetics described herein are gap junction channel inhibitors. In one embodiment, the gap junction comprises a connexin43 protein. In one embodiment, the gap junction comprises a connexin45 protein. In another embodiment, the peptides and peptide mimetics described herein are inhibitors of gap junction hemichannel opening. In one embodiment, the gap junction hemichannel comprises a connexin43 protein. In one embodiment, the gap junction hemichannel comprises a connexin45 protein. In other embodiments, the gap junctions and hemichannels comprise connexin25, connexin26, connexin29 (connexin30.2), connexin30, connexin30.3, connexin31, connexin31.1, connexin31.3, connexin31.9, connexin32, connexin36, connexin37, connexin40, connexin40.1, connexin46, connexin46.6 (connexin47), connexin50, connexin58, connexin59, connexin62 proteins, and the peptides and peptide mimetics described herein are inhibitors of one or more of these gap junction channels and corresponding hemichannels.

In another aspect, the inventions relate to compounds to control the opening of surface-exposed gap junction hemichannels. In another aspect, the inventions relate to compounds for sustained control of hemichannel opening. In another aspect, the inventions relate to compounds for closing hemichannels. In yet another aspect, the inventions relate to compounds for modulating hemichannel opening.

In one aspect, the inventions provided herein include compounds. In another aspect, the inventions include compositions comprising or consisting essentially of one or more of those compounds, the basic and novel characteristic of the inventions being the ability to modulate gap junction and/or hemichannel function or activity as described herein. The compounds are useful for the treatment of gap junction- and/or hemichannel-related disorders.

Compounds of the invention, which in a non-limiting preferred embodiment are isolated or substantially pure, include ADCFLSRPTEKT (SEQ.ID.NO:1), VDCALSRPTEKT (SEQ.ID.NO:2), VDCFLARPTEKT (SEQ.ID.NO:3), VDCFLSRPAEKT (SEQ.ID.NO:4), VDCFLSRPTEKA (SEQ.ID.NO:5), CFLSRPTAKT (SEQ.ID.NO:6), CALSRPTEKT (SEQ.ID.NO:7), CFLARPTEKT (SEQ.ID.NO:8), CFLSRPAEKT (SEQ.ID.NO:9), and CFLSRPTEKA (SEQ.ID.NO:10). In any and all of the above peptides shown as SEQ.ID.NO:1-10, separately or together in any combination, Valine (V) can be substituted with Alanine (A), Isoleucine (I), Leucine (L), Methionine (M) and Phenylalanine (F); Aspartic Acid (Asp) can be substituted with Glutamic Acid (E); Cysteine (C) can be substituted with Serine (S); Phenylalanine (P) can be substituted with Alanine (A), Tyrosine (Y), Tryptophan (W), Leucine (L), Isoleucine (I) and Valine (V); Leucine (L) can be substituted with Isoleucine (I), Methionine (M) and Valine (V); Serine (S) can be substituted with Alanine (A), Isoleucine (I), Leucine (L) and Valine (V), preferably Alanine; Arginine (R) can be substituted with Lysine (L) or Glutamine (Q); Proline (P) cannot be substituted; Threonine (T) can be substituted with Alanine (A), Asparagine (N), Serine (S) and Glysine (G); Glutamic Acid (E) can be substituted with Alanine (A), Glutamine (Q) and Asparagine (N); Lysine (K) can be substituted with Arginine (R), all of which are SEQ.ID.NO:1-8 analogs. In any and all of the above peptides shown as SEQ.ID.NO:1-8, separately or together in any combination, any of the amino acids can be L-amino acids or D-amino acids, provided, however, that the peptide is not L-Val L-Asp L-Cys L-Phe L-Leu L-Ser L-Arg L-Pro L-Thr L-Glu L-Lys L-Thr (SEQ ID NO: 45). Any of the above peptides shown as SEQ.ID.NO:1-8 that contain D-amino acids are also SEQ.ID.NO:1-8 analogs.

Compounds of the invention, which in a non-limiting preferred embodiment are isolated or substantially pure, also include compounds according to the following Formula I:

R₁ Val Asp Cys X₁ Leu Ser Arg Pro X₂ X₃ Lys X₄ (SEQ ID NO: 73)

wherein R₁ is absent, or selected from the group consisting of H, acyl groups containing a linear or branched, saturated or unsaturated hydrocarbon chain from 1 to 20 carbon atoms, amides, carbamates, ureas, PEG, or hydroxyalkyl starch; Xi is Phe, or an amino acid selected from the group consisting of Ala, Ile, Leu, Val, dehydroalanine, aminoisobutyric acid, and/or Trp, Tyr, Met, Ile, Leu, and/or Ser, Asn, Cys, Gln, Thr, Asp, Glu, Arg, His, Lys, Pro, and/or Nva or Nle, and/or tert-butylglycine, phenylglycine, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, and N-methyl-lysine, and/or a D-enantiomer of any of the foregoing; X₂ is Thr, or an amino acid selected from the group consisting of Ser, Asn, Ala, Be, Leu, Val, dehydroalanine, aminoisobutyric acid, Asp, Glu, Lys, Met, His, Pro, Gln, Arg, Ser, Nva or Nle; X₃ is Glu, Ala, dehydroalanine, aminoisobutyric acid, Asp, Lys, Gln, Gly, His, Arg, Asn, Met, Pro, Ser, Thr, Nva or Nle; and X₄ is Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle; provided, however, X₁, X₂, X₃ and X₄ are not, respectively, Phe, Thr, Glu and Thr at the same time and the peptide R₁ Val Asp Cys X₁ Leu Ser Arg Pro X₂ X₃ Lys X₄ peptide (SEQ ID NO: 73) is not L-Val L-Asp L-Cys L-Phe L-Leu L-Ser L-Arg L-Pro L-Thr L-Glu L-Lys L-Thr (SEQ ID NO: 45).

The invention also includes compounds of Formula I wherein X₁ is Phe and one or more of X₂, X₃ and/or X₄ are not, respectively, Thr, Glu and Thr. The invention also includes compounds of Formula I wherein X₂ is Thr and one or more of X₁, X₃ and/or X₄ are not, respectively, Phe, Glu and Thr. The invention also includes compounds of Formula I wherein X₃ is Glu and one or more of X₁, X₂ and/or X₄ are not, respectively, Phe, Thr and Thr. The invention also includes compounds of Formula I wherein X₄ is Thr and one or more of X₁, X₂ and/or X₃ are not, respectively, Phe, Thr and Glu.

Compounds of the invention, which in a non-limiting preferred embodiment are isolated or substantially pure, also include compounds according to the following Formula II:

R₁ Y₁ Asp Cys X₁ Leu Z₁ Arg Pro X₂ X₃ Lys X₄ (SEQ ID NO: 74)

Wherein Y₁ is Val, Ala or an amino acid selected from the group consisting of Ile, Leu, Met, Phe, Thr, Cys, Tyr, dehydroalanine, aminoisobutyric acid, Nva, Nle, tert-butylglycine, phenylglycine, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, and N-methyl-lysine, and/or a D-enantiomer of any of the foregoing; Z₁ is Ser, Ala or an amino acid selected from the group consisting of Asn, Thr, Cys, Gly, Asp, Glu, Lys, Gln, Gly, Pro, His and Arg; and wherein R₁, X₁, X₂, X₃ and X₄ are as described above for Fomula I; provided, however, that Y₁, X₁, Z₁, X₂, X₃ and X₄ are not, respectively, Val, Phe, Ser, Thr, Glu and Thr at the same time and the peptide R₁ Y₁ Asp Cys X₁ Leu Z₁ Arg Pro X₂ X₃ Lys X₄ peptide (SEQ ID NO: 74) is not L-Val L-Asp L-Cys L-Phe L-Leu L-Ser L-Arg L-Pro L-Thr L-Glu L-Lys L-Thr (SEQ ID NO: 45). The invention also includes compounds of Formula II wherein Y₁ is Val, and one or more of X₁, Z₁, X₂, X₃ and/or X₄ are not, respectively, Phe, Ser, Thr, Glu and Thr. The invention also includes compounds of Formula I wherein X₁ is Phe and one or more of Y₁, Z₁, X₂, X₃ and/or X₄ are not, respectively, Val, Ser, Thr Glu and Thr. The invention also includes compounds of Formula I wherein X₂ is Thr and one or more of Y₁, X₁, Z₁, X₃ and/or X₄ are not, respectively, Val, Phe, Ser, Glu and Thr. The invention also includes compounds of Formula I wherein X₃ is Glu and one or more of Y₁, X₁, Z₁, X₂ and/or X₄ are not, respectively, Val, Phe, Ser, Thr and Thr. The invention also includes compounds of Formula I wherein X₄ is Thr and one or more of Y₁, X₁, Z₁, X₂ and/or X₃ are not, respectively, Val, Phe, Ser, Thr and Glu.

Other embodiments of the invention, which in a non-limiting preferred embodiment are isolated or substantially pure, also include the amino acid sequence IDCFISRPTEKT (SEQ ID NO:43) will be isoform specific and useful, for example, as a blocker for gap junction channels and/or as a hemichannel blocker for hemichannels composed of Connexin 45. The sequence and active analogs thereof, including those with groups such as R₁ described above, and others useful for example to increase molecular mass and shield them from proteolytic enzymes, will be particularly important for addressing cardiac diseases, disorders and conditions, including those characterized at least in part by vessel damage and/or leakage, and as treatments to address vessel dieback from myocardial infarction and heart attack.

Compounds of the invention, which in a non-limiting preferred embodiment are isolated or substantially pure, also include compounds according to the following Formula III:

R₁ Ile Asp Cys X₁ Ile Ser Arg Pro X₂ X₃ Lys X₄ (SEQ ID NO: 75)

wherein R₁ is absent, or selected from the group consisting of H, acyl groups containing a linear or branched, saturated or unsaturated hydrocarbon chain from 1 to 20 carbon atoms, amides, carbamates, ureas, PEG, or hydroxyalkyl starch; X₁ is Phe, or an amino acid selected from the group consisting of Ala, Ile, Leu, Val, dehydroalanine, aminoisobutyric acid, and/or Trp, Tyr, Met, Ile, Leu, and/or Ser, Asn, Cys, Gln, Thr, Asp, Glu, Arg, His, Lys, Pro, and/or Nva or Nle, and/or tert-butylglycine, phenylglycine, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, and N-methyl-lysine, and/or a D-enantiomer of any of the foregoing; X₂ is Thr, or an amino acid selected from the group consisting of Ser, Asn, Ala, Be, Leu, Val, dehydroalanine, aminoisobutyric acid, Asp, Glu, Lys, Met, His, Pro, Gln, Arg, Ser, Nva or Nle; X₃ is Glu, Ala, dehydroalanine, aminoisobutyric acid, Asp, Lys, Gln, Gly, His, Arg, Asn, Met, Pro, Ser, Thr, Nva or Nle; and X₄ is Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle; provided, however, X₁, X₂, X₃ and X₄ are not, respectively, Phe, Thr, Glu and Thr at the same time and the peptide R₁ Ile Asp Cys X₁ Ile Ser Arg Pro X₂ X₃ Lys X₄ peptide (SEQ ID NO: 75) is not L-Ile L-Asp L-Cys L-Phe L-Ile L-Ser L-Arg L-Pro L-Thr L-Glu L-Lys L-Thr(SEQ ID NO: 43), or any naturally occurring connexin peptide sequence.

The invention also includes compounds of Formula III wherein X₁ is Phe and one or more of X₂, X₃ and/or X₄ are not, respectively, Thr, Glu and Thr. The invention also includes compounds of Formula III wherein X₂ is Thr and one or more of X₁, X₃ and/or X₄ are not, respectively, Phe, Glu and Thr. The invention also includes compounds of Formula III wherein X₃ is Glu and one or more of X₁, X₂ and/or X₄ are not, respectively, Phe, Thr and Thr. The invention also includes compounds of Formula III wherein X₄ is Thr and one or more of X₁, X₂ and/or X₃ are not, respectively, Phe, Thr and Glu.

Compounds of the invention, which in a non-limiting preferred embodiment are isolated or substantially pure, also include compounds according to the following Formula IV:

R₁ Y₁ Asp Cys X₁ ILe Z₁ Arg Pro X₂ X₃ Lys X₄ (SEQ ID NO: 76)

Wherein Y₁ is Be, Val, Leu, Ala, Met, and/or Phe or an amino acid selected from the group consisting of Ile, Leu, Met, Phe, Thr, Cys, Tyr, dehydroalanine, aminoisobutyric acid, Nva, Nle, tert-butylglycine, phenylglycine, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, and N-methyl-lysine, and/or a D-enantiomer of any of the foregoing; Z₁ is Ser, Ala or an amino acid selected from the group consisting of Asn, Thr, Cys, Gly, Asp, Glu, Lys, Gln, Gly, Pro, His and Arg; and wherein R₁, X₁, X₂, X₃ and X₄ are as described above for Fomula III; provided, however, that Y₁, X₁, Z₁, X₂, X₃ and X₄ are not, respectively, Val, Phe, Ser, Thr, Glu and Thr at the same time and the peptide R₁ Y₁ Asp Cys X₁ Ile Z₁ Arg Pro X₂ X₃ Lys X₄ peptide (SEQ ID NO: 76) is not L-Ile L-Asp L-Cys L-Phe L-Ile L-Ser L-Arg L-Pro L-Thr L-Glu L-Lys L-Thr (SEQ ID NO: 43), or any naturally occurring connexin peptide sequence.

Compounds of the invention, which in a non-limiting preferred embodiment are isolated or substantially pure, may also include or exclude 12 amino peptides according to Table 1.

Other embodiments of the invention, which in a non-limiting preferred embodiment are isolated or substantially pure, also include the amino acid sequence VDCFVSRPTEKT (SEQ ID NO:31), which is useful, for example, as a blocker for gap junction channels and/or hemichannels composed of any of connexins 50, 62, 37, 46.6, 26, 32 or 31.9; the amino acid sequence VDCFISRPTEKT (SEQ ID NO:32), which is useful, for example, as a blocker for gap junction channels and/or hemichannels composed of any of connexins 46 or 30.

Other embodiments of the invention, which in a non-limiting preferred embodiment are isolated or substantially pure, also include the amino acid sequence ITCNLSRPSEKT (SEQ ID NO:33), which is useful, for example, as a blocker for gap junction channels and/or hemichannels composed of any of connexins 29 (30.2) or 31.3; the amino acid sequence VDCYVSRPTEKS (SEQ ID NO:34), which is useful, for example, as a blocker for gap junction channels and/or hemichannels composed of connexin40.1; the amino acid sequence VECYVSRPTEKT (SEQ ID NO:35), which is useful, for example, as a blocker for gap junction channels and/or hemichannels composed of connexin36; the amino acid sequence IDCFVSRPTEKT (SEQ ID NO:36), which is useful, for example, as a blocker for gap junction channels and/or hemichannels composed of connexin 59; the amino acid sequence VDCYISRPTEKK (SEQ ID NO:37), which is useful, for example, as a blocker for gap junction channels and/or hemichannels composed of connexin 30.3; the amino acid sequence VDCFISKPSEKN (SEQ ID NO:38), which is useful, for example, as a blocker for gap junction channels and/or hemichannels composed of connexin 31.1; the amino acid sequence VDCYIARPTEKK (SEQ ID NO:39), which is useful, for example, as a blocker for gap junction channels and/or hemichannels composed of connexin 31; the amino acid sequence VDCFISKPTEKT (SEQ ID NO:40), which is useful, for example, as a blocker for gap junction channels and/or hemichannels composed of connexin 25; and, the amino acid sequence IDCFVSRPTEKT (SEQ ID NO:41), which is useful, for example, as a blocker for gap junction channels and/or hemichannels composed of any of connexin58 and connexin59.

Other embodiments of the invention, which in a non-limiting preferred embodiment are isolated or substantially pure, also include the amino acid sequence VNCYVSRPTEKN (SEQ ID NO:42) will be isoform specific and useful, for example, as a blocker for gap junction channels and/or as a hemichannel blocker for hemichannels composed of Connexin40. The sequence and active analogs thereof, including those with groups such as Ri described above, and others useful for example to increase molecular mass and shield them from proteolytic enzymes, will be particularly important for addressing vascular diseases, disorders and conditions, including those characterized at least in part by microvessel leakage, and as cancer treatments.

In certain embodiments, Ri can be a cellular internalization transporter. The cellular internalization transporter of the present invention may be any internalization sequence known or newly discovered in the art, or conservative variants thereof. Non-limiting examples of cellular internalization transporters and sequences include Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (BisGuanidinium-Tren-Cholesterol).

The sequences of exemplary cellular internalization peptides are provided in Table 4 below.

TABLE 4

| SEQ ID NO. | Identifier | Sequence |
|---|---|---|
| SEQ ID NO: 54 | ANTP | RQPKIWFPNRRKPWKK |
| SEQ ID NO: 55 | HIV-TAT | GRKKRRQRPPQ |
| SEQ ID NO: 56 | Transportan | GWTLNSAGYLLGKINKALAALAKKIL |
| SEQ ID NO: 57 | Buforin II | TRSSRAGLQFPVGRVHRLLRK |
| SEQ ID NO: 58 | Tat | RKKRRQRRR |
| SEQ ID NO: 59 | Penetratin | RQIKIWFQNRRMKWKK |
| SEQ ID NO: 60 | MAP | KLALKLALKALKAALKLA |
| SEQ ID NO: 61 | K-FGF | AAVALLPAVLLALLAP |
| SEQ ID NO: 62 | Ku70 | VPMLKPMLKE |
| SEQ ID NO: 63 | Prion | MANLGYWLLALFVTMWTDVGLCKKRPKP |
| SEQ ID NO: 64 | pVEC | LLIILRRRIRKQAHAHSK |
| SEQ ID NO: 65 | Pep-1 | KETWWETWWTEWSQPKKKRRV |
| SEQ ID NO: 66 | SynB1 | RGGRLSYSRRRFSTSTGR |
| SEQ ID NO: 67 | Pep-7 | SDLWEMMMVSLACQY |
| SEQ ID NO: 68 | HN-1 | TSPLNIHNGQKL |
| SEQ ID NO: 69 | plsl | RVIRVWFQNKRCKDKK |
| SEQ ID NO: 70 | MGB Peptide P- | GALFLGFLGAAGSTMGAWSQPKKKRKV |
| | beta | |
| SEQ ID NO: 71 | MGB Peptide P- | GALFLAFLAAALSLMGLWSQPKKKRRV |
| | alpha | |

Table 4 lists sequences of exemplary cellular internalization transporters.

In some embodiments, the connexin, pannexin and/or cadherin modulator peptide is fused to a transport peptide to increase the penetration into the target cell. In some embodiments, the transport peptide can be part of a viral coating for cell penetration. In some embodiments, the transport peptide can be fused to the connexin and/or cadherin modulator peptide at the carboxy or amino terminus. The transport peptide can be selected from one of the following peptides: ANTP, HIV-TAT, Transportan, Buforin II, Tat, Penetratin, MAP, K-FGF, Ku70, Prion, pVEC, Pep-1, SynB1, Pep-7, RGD, or HN-1.

In one embodiment of the present invention, the amino acid sequence of the connexin 43 modulator peptides can be selected from the group consisting of any peptide SEQ ID listed herein, or a conservative variant thereof. In a further embodiment of the present invention, the connexin 43 modulator peptides can comprise the amino acid sequence of SEQ ID NO: 54-71. In another embodiment of the present invention, the connexin 43 modulator peptide further comprises a cellular internalization transporter. In a further embodiment, the connexin 43 modulator peptide can be linked at the amino terminus to the cellular internalization transporter.

Other embodiments of the invention include combination treatments with peptides matching regions of both connexin extracellular loops 1 and 2 show increased efficacy (FIG. 3, Table 2), exceeding the efficacy of carbenoxolone commonly used as a gold standard control channel blocker.

Other embodiments of the invention include compositions and treatments comprising a combination of the connexin hemichannel blocker VCYDKSFPISHVR (SEQ ID NO:44) with any one or more of the peptides described herein, including those according to Formula I or Formula II, will also be useful as a connexin43 hemichannel blocking combination.

These sequences may be delivered separately or together as independent entities or linked. Thus, the invention also includes, for example, the connexin43 hemichannel blocker VCYDKSFPISHVR (SEQ ID NO:44) linked to the connexin43 hemichannel blocker VDCFLSRPTEKT (SEQ ID NO:45) in the form, for example, VDCFLSRPTEKT-X-VCYDKSFPISHVR (SEQ ID NO:46) where X is one or more linking glycines or other non-functional linker with sufficient flexibility for the combination peptide to interact with both of their binding sites respectively on connexin43 hemichannels.

The invention also includes, for example, sequences in combination that are functional against multiple connexin isoforms in a tissue at the same time, and may be delivered together separately or together as independent entities or linked. For example, the connexin43 hemichannel blocker VDCFLSRPTEKT (SEQ ID NO:47) can be linked to the connexin40 hemichannel blocker VNCYVSRPTEKN (SEQ ID NO:48) in the form VDCFLSRPTEKT-X-VNCYVSRPTEKN (SEQ ID NO:49) where X is one or more linking glycines or other non-functional linker enabling the combination peptide to interact with hemichannels composed of Connexin43, Connexin40, or Connexin45.

The invention also includes, for example, the or four peptide connexin isoform blocker sequences in combination, and which do not form tertiary structures and the linking provides for retention of linear structure. In one embodiment, a peptide for vascular endothelial connexins 40, 43, 45 and 37 is prepared, including: VDCFVSRPTEKN (SEQ ID NO:50); VDCFLSRPTEKN (SEQ ID NO:51); and VDCFVSRPTEKT (SEQ ID NO:52).

Included in the scope of the invention are active analogs and conservative variants of these compounds, including truncations thereof, preferably N-terminal truncations of 1 or 2 amino acids.

In one non-limiting embodiment, one or more of the amino acids of the peptides within the scope of the invention, including SEQ.ID.NOS:1-52 and sequences within Formulae I-IV, may be in the L- or D-configuration. In other embodiments, one or more of the amino acids of the peptides within the scope of the invention are naturally-occuring non-genetically coded amino acids. In still other embodiments, one or more of the amino acids of the peptides within the scope of the invention are amino acid analogs or synthetic amino acids.

In another non-limiting embodiment, the N-terminal amino acid may be modified to contain a formyl group, a group comprising a formyl group, an ester of a carboxylic acid (preferably an aldehyde ester, e.g., a carboxyethyl group, a carboxymethyl group, etc.), or a group comprising an ester of a carboxylic acid. Modifications with formyl, carboxyethyl, and carboxymethyl groups are presently preferred.

In another embodiment, one or more the amino acids in compounds within the scope of the invention, including SEQ.ID.NOS:1-52 and sequences within Formulae I-IV, are substituted for another amino acid from a similar amino acid class or subclass, based primarily upon the chemical and physical properties of the amino acid side chain. For example, one or more hydrophilic or polar amino acids can be substituted for another hydrophilic or polar amino acid. Likewise, one or more hydrophobic or nonpolar amino acids can be substituted for another hydrophobic or nonpolar amino acid. In making such substitutions, polar amino acids can be further subdivided into amino acids having acidic, basic or hydrophilic side chains and nonpolar amino acids can be further subdivided amino acids having aromatic or hydrophobic side chains. Nonpolar amino acids may be further subdivided to include, among others, aliphatic amino acids.

Also within the scope of the invention are compounds of the invention that have been modified to improve their biopharmaceutical properties. In certain embodiments, the compounds of the invention are modified, for example, to provide increased stability, increased resistance to proteolytic inactivation, decreased to nonexistent immunogenicity, increased circulatory lives, including modified serum half-lives and modified therapeutic half-lives, and low toxicity. Modified forms of compounds of the invention include prodrug forms, representative examples of which are described elsewhere herein. Methods by which the compounds of the invention can be modified also include, for example, by PEGylation, by chemical derivitization, and by fusion or conjugation with peptides or lipids. Modifided compounds include modified peptide hemichannel agents, including, for example, any of modified SEQ ID NOS:1-10, and modified analogs, and variants (e.g., conservative variants) thereof.

Other embodiments include peptides selected from SEQ.ID.NOS:1 to 10, and peptides according to any of Formula I, Formula II, Formula III and/or Formula IV, that do not include a C-terminal Threonine. Other embodiments include peptides selected from SEQ.ID.NOS:1 to 10, and peptides according to Formula I and/or Formula II, that do not include an N-terminal Valine. Other embodiments include peptides according to Formula III and/or Formula IV, that do not include an N-terminal Isoleucine. Other embodiments include peptides selected from SEQ.ID.NOS:1 to 10, and peptides according to Formula I and/or Formula II, that do not include an N-terminal Valine or a C-terminal Threonine. Other embodiments include peptides according to Formula III and/or Formula IV, that do not include an N-terminal Isoleucine or a C-terminal Threonine.

Other embodiments include peptides selected from SEQ.ID.NOS:11 to 52 that have the C-terminal amino acid removed, i.e., a single C-terminal truncation. Other embodiments include peptides selected from SEQ.ID.NOS:11 to 52 that have the first and/or second N-terminal amino acid removed, i.e., a single or double N-terminal truncation.

In some embodiments, present invention provides a pharmaceutical composition comprising one or more of the peptides or peptide mimetics of the present invention, and a pharmaceutically acceptable carrier or excipient. In some embodiments, present invention provides a kit for the prophylaxis or treatment of a mammal for one or more of the diseases, disorders or conditions described or referenced herein, characterized in that said kit comprises one or more gap junction channel and/or hemichannel blockers or inhibitors, optionally with reagents and/or instructions for use, wherein said one or more gap junction channel and/or hemichannel blockers or inhibitors comprise a sequence of at least 10 contiguous amino acids of any of the peptides or peptide mimetics described herein.

The present inventions also include pharmaceutical compositions comprising or consisting essentially of the peptide fragment agent and a pharmaceutically acceptable carrier and one or more of the gap junction channel and/or hemichannel blockers or inhibitor compounds described or referenced herein. In one embodiment, the pharmaceutical composition comprises or consists essentially of any of SEQ ID NOS:1-52. In another embodiment, the pharmaceutical composition comprises or consists essentially of a sequence selected from either of Formula I or Formula II. Included in the scope of the invention are pharmaceutical compositions including one or more active analogs and conservative variants of these compounds, including truncations thereof, preferably 1- or 2-amino acid N-terminal truncations as described, or a 1-amino acid C-terminal truncation.

In another embodiment, the inventions include pharmaceutical compositions comprising or consisting essentially of compounds of the invention, including analogs, variants, truncations, etc., that have been modified to improve their biopharmaceutical properties. In certain embodiments, the compounds of the invention are modified, for example, to provide increased stability, increased resistance to proteolytic inactivation, decreased to nonexistent immunogenicity, increased half-lives or circulatory lives, and low toxicity. Methods by which the compounds of the invention can be modified include, for example, by PEGylation, by chemical derivitization, and by fusion or conjugation with peptides or lipids.

The inventions include a pharmaceutical composition comprising one or more pharmaceutically acceptable gap junction channel and/or hemichannel blockers or inhibitor compounds described or referenced herein for the treatment of a cardiovascular disorder, e.g., an acute coronary syndrome, heart failure, ischemic heart disease, etc., and related cardiovascular diseases, disorders and conditions characterized at least in party by ischemia and/or oxidative stress, and related disorders and conditions. Certain preferred gap junction channel and/or hemichannel blockers or inhibitor compounds are identified herein as SEQ.ID.NOS:1 to 52. SEQ.ID.NOS:1-10 are particularly preferred connexin43 gap junction channel and/or hemichannel blockers or inhibitor compounds. Other particularly preferred gap junction channel and/or hemichannel blockers or inhibitor compounds for other connexins are described.

The inventions include pharmaceutical compositions in a form suitable for, or adapted to, treatment of a subject for a cardiovascular disease, disorder or condition. The inventions include pharmaceutical compositions in a form suitable for, or adapted to, treatment of a subject for an ocular disease, disorder or condition, including retinal diseases, disorders and conditions, and other ocular diseases, disorders and conditions characterized in whole or in part by vasucular damage or leak, including for example diseases, disorders and conditions characterized by choroidal neovascularization, damage, leak, edema and/or inflammation.

In one embodiment, the present invention provides connexin gap junction or connexin hemichannel compounds that act as connexin gap junction or connexin hemichannel antagonists, or blockers. In one embodiment, the present invention provides methods for treating angiogenic eye disorders by sequentially administering multiple doses of a connexin gap junction or connexin hemichannel antagonist or blocker to a patient. The methods of the present invention include the administration of multiple doses of a connexin gap junction or connexin hemichannel antagonist or blocker to a patient at a frequency of once every 6, 7, 8 or more weeks.

The methods of the present invention are useful for the treatment of angiogenic eye disorders such as age related macular degeneration, diabetic retinopathy, diabetic macular edema, central retinal vein occlusion, branch retinal vein occlusion, and corneal neovascularization.

The present invention provides methods that comprise sequentially administering multiple doses of a connexin gap junction or connexin hemichannel antagonist or blocker to a patient over time. In particular, the methods of the invention comprise sequentially administering to the patient a single initial dose of a connexin gap junction or connexin hemichannel antagonist or blocker, followed by one or more secondary doses of the connexin gap junction or connexin hemichannel antagonist or blocker, followed by one or more tertiary doses of the connexin gap junction or connexin hemichannel antagonist or blocker. Thus, according to the methods of the present invention, each secondary dose of connexin gap junction or connexin hemichannel antagonist or blocker is administered 2 to 4 weeks after the immediately preceding dose, and each tertiary dose is administered at least 8 weeks after the immediately preceding dose. An advantage of such a dosing regimen is that, for most of the course of treatment (i.e., the tertiary doses), it allows for less frequent dosing (e.g., once every 8 weeks) compared to prior administration regimens for angiogenic eye disorders which require monthly administrations throughout the entire course of treatment. See, e.g., prescribing information for Lucentis® [ranibizumab], Genentech, Inc.

The expression “angiogenic eye disorder,” as used herein, means any disease of the eye which is caused by or associated with the growth or proliferation of blood vessels or by blood vessel leakage. Non-limiting examples of angiogenic eye disorders that are treatable using the methods of the present invention include age-related macular degeneration (e.g., wet AMD, exudative AMD, etc.), retinal vein occlusion (RVO), central retinal vein occlusion (CRVO; e.g., macular edema following CRVO), branch retinal vein occlusion (BRVO), diabetic macular edema (DME), choroidal neovascularization (CNV; e.g., myopic CNV), iris neovascularization, neovascular glaucoma, post-surgical fibrosis in glaucoma, proliferative vitreoretinopathy (PVR), optic disc neovascularization, corneal neovascularization, retinal neovascularization, vitreal neovascularization, pannus, pterygium, vascular retinopathy, and diabetic retinopathies.

In one embodiment, the disease, disorder or condition is associated with inflammation, e.g., vascular or microvascular inflammation. In another embodiment, the disease, disorder or condition is associated with ischemia and/or oxidative stress, e.g., vascular or microvascular ischemia and/or oxidative stress. In another embodiment, the disease, disorder or condition is associated with edema, e.g., edema associated with vascular or microvascular damage or leak.

In one embodiment, the disease, disorder or condition is associated with a neoplasm, i.e., an abnormal mass of tissue that results when cells divide more than they should or do not die when they should. As used herein, neoplasms are also referred to as tumors and may be benign (not cancer) malignant (cancer).

In certain embodiments, the cardiovascular disease, disorder or condition is an acute coronary syndrome. The acute coronary syndrome may, for example, be selected from the group consisting of ST-segment elevation myocardial infarction, non-ST-segment elevation myocardial infarction and unstable angina. In other embodiments, the cardiovascular disease, disorder or condition is ischemic heart disease. In other embodiments, the cardiovascular disease, disorder or condition is heart failure (any form). For example, the heart failure may be systolic or diastolic heart failure. The heart failure may result from left ventricular systolic dysfunction. The heart failure may also be a result of right ventricular infarction, pulmonary hypertension, chronic severe tricuspid regurgitation, or arrhythmogenic right ventricular dysplasia. The heart failure may also be a result of diastolic LV dysfunction. In another embodiment the cardioiovascular disease, disorder or condition is ischemic heart disease. Connexin40, 43 and 45 gap junction channel and/or hemichannel blockers or inhibitor compounds are particularly useful for treatment of a cardiovascular disease, disorder or condition.

In one aspect, the invention includes pharmaceutical compositions useful for preventing and/or treating a subject for one or more of the disease, disorder or condition described or referenced herein a subject, including parenteral delivery forms and formulations, as well as other forms of delivery including forms for delivery by infusion, injection and instillation, and delayed, slow, extended or controlled release compositions, devices and matrices, comprising or consisting essentially of therapeutically effective amounts of a gap junction channel and/or hemichannel blocker or inhibitor compound alone or in combination with another cardiovascular therapeutic agent(s), and a pharmaceutically acceptable carrier. In certain preferred embodiments, the pharmaceutical compositions are formulated for intravenous administration, including by infusion or as a bolus. Other formulations for other routes of administration are also within the scope of the invention, including, for example, formulations for nasal, pulmonary, buccal, rectal, transdermal and oral delivery.

In another aspect, the compositions of the invention comprise about 0.01 to about 100 milligrams, about 100 to about 500 milligrams, or about 500 to about 1000 milligrams or more of a compound of the invention, for example, a gap junction channel and/or hemichannel blocker or inhibitor compound or analog, including one or more of SEQ.ID.NOS:1-52 and peptides according to any of Formulae I to II. Other doses are described herein and include doses ranging from at least about 100 nanograms, including, for example at least about 200 micrograms or milligrams, 600 micrograms or milligrams, 2000 micrograms or milligrams, 6000 micrograms or milligrams and at least about 10,000 micrograms or milligrams. Dose concentrations include concentrations of at least about 0.1 moles per liter, including, for example, at least about 0.3, 1.0, 3.0 and 10.0 Moles/L. Dose concentrations also include concentrations of 0.1 micromoles or millimoles/L, 0.3 micromoles or millimoles/L, 1.0 micromoles or millimoles/L, 3.0 micromoles or millimoles/L and 10.0 micromoles or millimoles/L. Dose concentrations may be equivalent to 0.1, 0.3, 1, 3, and 10 to 100 mg/mL and administrable weight doses of 1-10, 10-20, 20-50, 50-100, 100-500 and 500-1000 milligrams/kg (mg/kg). Also within the invention are other doses ranging from 0.1 to 5.0 μg/kg and 0.1 to 10.0 g/kg. These compositions and amounts may be provided as single or muliple doses.

The gap junction channel and/or hemichannel blocker or inhibitor compound (or pharmaceutical formulation comprising the gap junction channel and/or hemichannel blocker or inhibitor compound) may be administered to the patient by any known delivery system and/or administration method. In certain embodiments, the gap junction channel and/or hemichannel blocker or inhibitor compound is administered to the patient by ocular, intraocular, intravitreal or subconjunctival injection. In other embodiments, the gap junction channel and/or hemichannel blocker or inhibitor compound can be administered to the patient by topical administration, e.g., via eye drops or other liquid, gel, ointment or fluid which contains the gap junction channel and/or hemichannel blocker or inhibitor compound and can be applied directly to the eye. Other possible routes of administration include, e.g., intradermal, intramuscular, intraperitoneal, intravenous, subcutaneous, intranasal, epidural, and oral.

The inventions also include methods of treatment of a subject having or at risk for developing a disease, disorder or condition as referenced herein, comprising administering to the subject a therapeutically effective amount of one or more of the compounds or pharmaceutical compositions described herein. In one non-limiting embodiment, the disease, disorder or condition is associated with vessel damage, vessel leak, edema, inflammation, ischemia and/or oxidative stress. In one embodiment, the disease, disorder or condition is an acute coronary syndrome, e.g., ST-segment elevation myocardial infarction, non-ST-segment elevation myocardial infarction or unstable angina. In another embodiment, the cardiovascular disease, disorder or condition is heart failure. In other embodiments, the cardiovascular disease, disorder or condition is ischemic heart disease. In another embodiment, the cardiovascular disease, disorder or condition is stable angina.

The inventions include methods of treating a subject having or at risk for developing, or at risk for progression of, a disease, disorder or condition described or referenced herein, comprising a therapeutically effective amount of a gap junction channel and/or hemichannel blocker or inhibitor compound and a pharmaceutically acceptable carrier. In one embodiment, the gap junction channel and/or hemichannel blocker or inhibitor compound in the pharmaceutical composition comprises or consists essentially of a sequence selected from SEQ.ID.NOS:1-52. In another embodiment, the gap junction channel and/or hemichannel blocker or inhibitor compound in the pharmaceutical composition comprises or consists essentially of a sequence selected from Formula I or Formula II. Gap junction channel and/or hemichannel blocker or inhibitor compounds also include active analogs, variants, truncations, and modified forms of the gap junction channel and/or hemichannel blocker or inhibitor compounds described herein.

In another aspect, the inventions include methods of treating and/or preventing a cardiovascular or ocular disease, disorder or condition that is associated with inflammation, ischemia and/or oxidative stress in a subject by decreasing gap junction channel and/or hemichannel activity in the subject. This may be accomplished, for example, by administering to the subject a composition comprising a therapeutically effective amount of a gap junction channel and/or hemichannel blocker or inhibitor compound, e.g., a gap junction channel and/or hemichannel blocker or inhibitor compound comprising or consisting essentially of a sequence selected from SEQ.ID.NOS:1-52, or a peptide comprising or consisting essentially of a peptide according to any of Formulae I to II, or an analog, variant, truncation or modification thereof, as described for example. In certain embodiments, about 0.01 to about 100, 500 or 1000 nanograms or milligrams or more (e.g., at least about 100 nanograms or milligrams, at least about 500 nanograms or milligrams, or at least about 1000 nanograms or milligrams) of a gap junction channel and/or hemichannel blocker or inhibitor compound or analog is administered per day in single or divided doses or by continuous or periodic release, for example.

In one embodiment, the gap junction channel and/or hemichannel blocker or inhibitor compound is administered in a single dose. In another embodiment, the gap junction channel and/or hemichannel blocker or inhibitor compound is administered in more than one dose. In yet another embodiment, the gap junction channel and/or hemichannel blocker or inhibitor compound is administered or released continuously over a period of time, for example a predetermined period of time. In still another embodiment, a cardiovascular treatment agent or an ocular medicine (e.g., a VEGF antagonist) is administered or co-administered with the gap junction channel and/or hemichannel blocker or inhibitor compound. Such other cardiovascular treatment agents include nitrates, β-blockers, calcium channel blockers (particularly for stable or unstable angina, but also for heart failure in the case of β-blockers), diuretic agents, vasodilator agents, positive inotropes, ACE inhibitors and aldosterone antagonists, e.g. spironolactone (particularly for heart failure), blood thinning therapeutics (e.g., aspirin, heparins, warfarins) and nitroglycerin (particularly for MI).

The inventions also provide a method for performing angioplasty on a patient in need thereof, comprising administering a gap junction channel and/or hemichannel blocker or inhibitor compound to the patient during the angioplasty procedure. In a further embodiment, the method comprises or further comprises administering a gap junction channel and/or hemichannel blocker or inhibitor compound to the patient prior to the angioplasty procedure. In a further embodiment, the method comprises or further comprises administering a gap junction channel and/or hemichannel blocker or inhibitor compound to the patient following the angioplasty procedure. In other embodiments, a gap junction channel and/or hemichannel blocker or inhibitor compound is administerd to the patient before, during, and/or after the angioplasty procedure, in any combination.

Also provided are methods for increasing the time during which thrombolytic therapy will be effective following the first symptom of cardiac distress, comprising administering a therapeutically effective amount of a gap junction channel and/or hemichannel blocker or inhibitor compound after the onset of one or more of the following symptoms: chest pain lasting longer than 15 minutes, chest pain at rest, chest pain following minimal exertion, nausea, shortness of breath, palpitations, or dizziness.

In another aspect, the treated subject is a mammal, preferably a human. Other mammals include domestic and farm animals, and zoo, sports, or pet animals, such as dogs, horses, and cats.

The inventions also include articles of manufacture comprising package material containing one or more of the compounds or pharmaceutical compositions described herein. Then inventions also include articles of manufacture comprising package material containing one or more of the compounds or pharmaceutical compositions described herein, together with instructions for use in or on a subject in order to prevent and/or treat a disease, disorder or condition as noted herein. In one embodiment, a vascular disease, disorder or condition is referred to in the instructions that is associated with inflammation, angiogeneisis and/or vessel leak, ischemia and/or oxidative stress. In another embodiment the disease, disorder or condition is referred to in the instructions is a cardioiovascular disease, disorder or condition. In one embodiment, the disease, disorder or condition referred to in the instructions is an angiogenic eye disorder. The instructions may be electronic and/or associated with a website.

The inventions also include methods of preparing a medicament for preventing or treating one or more of the diseases, disorders or conditions referenced herein, comprising bringing together a therapeutically effective amount of a compound referenced herein, e.g., a gap junction channel and/or hemichannel blocker or inhibitor compound or analog or variant, and a pharmaceutically acceptable carrier. In one embodiment the gap junction channel and/or hemichannel blocker or inhibitor compound comprises a sequence selected from SEQ.ID.NOS:1 to 52. In another embodiment the gap junction channel and/or hemichannel blocker or inhibitor compound is a compound selected from one or more of Formulae I-II. In one embodiment the medicament is formulated for parenteral administration. In one embodiment the medicament is formulated for ocular administration. In another embodiment the medicament is formulated for topical administration. In another embodiment the medicament is formulated for oral administration.

Treatment of a subject as provided herein with one or more compounds or pharmaceutical compositions as described herein may comprise their simultaneous, separate, sequential or sustained administration.

Pharmaceutical compositions useful for preventing and/or treating a disease, disorder or conditioned referenced or described herein are also provided in the form of a combined preparation, for example, as an admixture of two or more gap junction channel and/or hemichannel blocker or inhibitor compounds, analogs or variants.

The term “a combined preparation” includes not only physical combinations of compounds, but compounds provided as a “kit of parts” in the sense that the combination partners as defined above can be dosed independently or by use of different fixed combinations with distinguished amounts of the combination partners (a) and (b), i.e. simultaneously, separately or sequentially. The parts of the kit can then, for example, be administered simultaneously or chronologically staggered, that is at different time points and with equal or different time intervals for any part of the kit of parts.

In another aspect, the invention includes methods for administering a therapeutically effective amount of a gap junction channel and/or hemichannel blocker or inhibitor compound or analog or variant, alone or in combination with another therapeutic agent, formulated in a delayed release preparation, a slow release preparation, an extended release preparation, a controlled release preparation, and/or in a repeat action preparation to a subject having or at risk for developing a disease, disorder or condition described or referenced herein.

In certain other aspects, the invention also relates to methods of using such compositions to treat subjects suffering from or at risk for said diseases, disorders and conditions.

In other aspects, the inventions include methods and compositions for preventing and/or treating a subject having or suspected of having or predisposed to, or at risk for, any diseases, disorders and/or conditions characterized in whole or in part by vascular damage or leak.

According to one aspect, the present invention is directed to methods of halting or decreasing or providing relief from the symptoms of a cardiovascular or ocular disorder.

The invention includes an article of manufacture comprising packaging material containing one or more dosage forms as described herein, wherein the packaging material has a label that indicates that the dosage form can be used for a subject having or suspected of having or predisposed to any of the diseases, disorders and/or conditions described or referenced herein.

The invention includes methods for the use of a therapeutically effective amount of a gap junction channel and/or hemichannel blocker or inhibitor compound(s) in the manufacture of a dosage form useful for preventing and/or treating a cardiovascular or ocular disorder. Such dosage forms include, for example, oral delivery forms and formulations, well as other forms of delivery including forms for delivery by infusion, injection and instillation, and compositions and devices including slow-release, extended release, and delayed release compositions, depots and matrices, for example. Such dosage forms include those for the treatment of a subject as disclosed herein.

Also within the scope of the invention are compounds of the invention that have been modified to improve their biopharmaceutical properties. In certain embodiments, the compounds of the invention are modified, for example, to provide increased stability, increased resistance to proteolytic inactivation, decreased to nonexistent immunogenicity, increased circulatory lives, including modified serum half-lives and modified therapeutic half-lives, and low toxicity. Methods by which the compounds of the invention can be modified include, for example, by PEGylation, by chemical derivitization, and by fusion or conjugation with peptides or lipids. Modifided compounds include modified gap junction channel and/or hemichannel blocker or inhibitor compounds.

This invention envisions prodrug forms of the therapeutic peptides of the invention. A “prodrug” is a modified form of a therapeutic peptide that includes a reversible chemical modification that can reliably removed to convert the prodrug to the parent peptide through either an enzymatic or nonenzymatic catalytic reaction under physiological conditions following delivery to a patient. Such modifications can enhance chemical stability, alter aqueous solubility, extend biological half-life, broaden therapeutic indices, improve pharmacodynamics, and/or improve bioavailability, for example, while preserving the pharmacological properties of the parent therapeutic peptide. Such modifications can also allow the parent peptide to be released after it reaches the biological compartment where it can exert the desired effect. A “prodrug” is a compound that may include one or more specialized non-toxic protective groups used in a transient manner to alter or to eliminate certain limiting properties in the parent peptide, which protective group(s) can be removed by enzymatic or chemical cleavage. Any suitable protective group(s) can be employed to generate a peptide prodrug of the invention. Such specialized modifications include inclusion of one or more amino acid residues at either or both the amino- and/or carboxy-terminus of the parent peptide. Cleavage sites that allow for the efficient in vivo removal of additional N- or C-terminal amino acids or amino acid sequences are preferably included in such prodrug molecules. Modifications other than the addition of one or more N- and/or C-terminal amino acid residues are also envisioned. For example, diketopiperazine and diketomorpholine (DKP and DMP) strategies for prodrug conversion may be used (see, e.g., Application of Peptide-Badsed Prodrug Chemistry in Drug Development, Springer, Ed. De, Arnab (2012)), where prodrugs slowly convert to the parent drug at physiological conditions driven by the compounds' inherent chemical instability, without the need of any enzymatic cleavage. To improve stability, parent peptides of the invention can be protected against exopeptidase-mediated hydrolysis by bioreversibly masking N- and/or C-terminal amino acids.

Examples of prodrugs of the invention are those wherein the parent peptide includes one or more additional amino acid residues appended to the N- and/or C-terminus of the parent peptide. The compounds of the invention also include prodrug forms of the agents of the invention. For example, prodrug forms include those having one to 16 amino acid residues appended to the N-terminus of, for example, peptides including SEQ.ID.Nos:1-10 or peptides of any of Formulae I-IV.

Further examples of a prodrug according to the invention include those wherein an amino group of parent peptide is acylated, alkylated, phosphorylated, eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated, and the like; a compound wherein a hydroxy group of the parent peptide is acylated, alkylated, phosphorylated, acetylated, palmitoylated, propanoylated, pivaloylated, succinylated, fumarylated, alanylated or dimethylaminomethylcarbonylated, and the like; and a compound wherein a carboxy group of the parent peptide is esterified or amidated (e.g., ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-exo-1,3-dioxolen-4-yl)methyl esterified, cyclohexyloxycarbonylethyl esterified or methylamidated, and the like) and the like.

Other prodrugs forms are also envisioned, including those containing chemical modifications to one or more amino acids residues that are not positioned at the N- or C-terminus of the parent peptide. As those in the art will appreciate, any suitable chemical modification that can be removed under physiological conditions to yield a pharmaceutically active form of a compound of the invention can be utilized.

Other embodiments include peptidiomimetics of compounds of the invention.

Illustrations of cardioprotective activities of gap junction channel and/or hemichannel blocker or inhibitor compounds are provided in the Examples.

Synthesis of Gap junction channel and/or hemichannel blocker or inhibitor compounds, as well as modifed Gap junction channel and/or hemichannel blocker or inhibitor compounds, is carried out using methods known in the art. Compounds of the inventions that are peptides, such as SEQ.ID.NOS:1-52, can be made by solid-state chemical peptide synthesis. Other compounds, such as fusion peptides, can also be made by conventional recombinant techniques using standard procedures described in, for example, Sambrook & Maniaitis. The peptides and other compounds of the invention may be chemically modified. This may enhance their resistance to peptidases and other enzymes, restrictclearance by the kidney, etc. Methods of preparing such modified compounds are known in the art.

The precise sequence of the gap junction channel and/or hemichannel blocker or inhibitor compounds used will depend upon its ability to ameliorate on or more of the symtoms or effects of a cardiovascular disorder. Means for determining such effects are provided in the Examples.

Suitable gap junction channel and/or hemichannel blocker or inhibitor compounds for the preparation of the pharmaceutical compositions of the invention include SEQ.ID.NO:1-52. Other suitable gap junction channel and/or hemichannel blocker or inhibitor compounds for the preparation of the pharmaceutical compositions of the invention include peptides within Formulae I-IV. Other suitable gap junction channel and/or hemichannel blocker or inhibitor compounds for the preparation of the pharmaceutical compositions are described herein, and include, for example, analogs, variation, truncations and modifications (including fusions) of the foregoing compounds.

Gap junction channel and/or hemichannel blocker or inhibitor compounds activity can be selected in terms of their sequence and desired activity by any convenient, and conventional, approach including, for example, as described in the Examples.

### Therapeutic Agents

Compositions and methods of the invention for the prevention and/or treatment of a disease, disorder or condition described or referenced herein, e.g., an acute coronary syndrome or angiogenic ocular disorder, and related diseases, disorders and conditions involving inflammation, ischemia and/or oxidative stress, also comprise administration of a gap junction channel and/or hemichannel blocker or inhibitor compound in series or in combination with (e.g., in physical combination, provided as a combined preparation) one or more other treatment agents. Cardiovascular therapeutic agents include nitrates, β-blockers, calcium channel blockers (particularly for stable or unstable angina, but also for heart failure in the case of β-blockers), diuretic agents, vasodilator agents, positive inotropes, ACE inhibitors and aldosterone antagonists, e.g. spironolactone (particularly for heart failure), blood thinning therapeutics (e.g., aspirin, heparins, warfarins) and nitroglycerin (particularly for MI). Ocular treatment agents include anti-VEGF compounds.

Treatment of a subject as provided herein with one or more compounds or pharmaceutical compositions as described herein may comprise their acute or sustained administration and, in the case of combinations, their simultaneous, separate, or sequential administration, as further described herein.

The agents of the invention of the may be administered to a subject in need of treatment, such as a subject with any of the diseases, disorders or conditions mentioned herein. The condition of the subject can thus be improved. The agents may be used in the manufacture of a medicament to treat any of the diseases, disorders or condtions mentioned herein. Thus, in accordance with the invention, there are provided formulations by which cardiovascular disorders can be treated.

The agents of the invention of the may be administered to a subject in need of treatment, such as a subject with an acute coronary syndrome or any of the diseases or conditions mentioned herein. The condition of the subject can thus be improved. The compounds may thus be used in the treatment of the subject's body by therapy. They may be used in the manufacture of a medicament to treat any of the conditions mentioned herein.

### Dosage Forms and Formulations and Administration

The compounds of the invention may be present in an isolated or substantially or essentially pure form. It will be understood that the product may be mixed with carriers or diluents which will not interfere with the intended purpose of the product and still be regarded as isolated or substantially pure. A product of the invention may also be in a substantially or essentialy purified form, preferably comprising or consisting essentially of about 80%, 85%, or 90%, e.g. at least about 95%, at least about 98% or at least about 99% of the compound or dry mass of the preparation.

Depending on the intended route of administration, the pharmaceutical products, pharmaceutical compositions, combined preparations and medicaments of the invention may, for example, take the form of solutions, suspensions, instillations, sustained release formulations, or powders, and typically contain about 0.1%-95% of active ingredient(s), preferably about 0.2%-70%. Other suitable formulations include injection- and infusion-based formulations. Other useful formulations include sustained release preparations, including, for example, controlled, slow or delayed release preparations.

Aspects of the invention include controlled or other doses, dosage forms, formulations, compositions and/or devices containing one or more gap junction channel and/or hemichannel blocker or inhibitor compounds. The present invention includes, for example, doses and dosage forms for at least oral administration, transdermal delivery, topical application, ocular delivery suppository delivery, transmucosal delivery, injection (including subcutaneous administration, subdermal administration, intramuscular administration, depot administration, and intravenous administration, including delivery via bolus, slow intravenous injection, and intravenous drip), infusion devices (including implantable infusion devices, both active and passive), administration by inhalation or insufflation, buccal administration and sublingual administration. It will be appreciated that any of the dosage forms, compositions, formulations or devices described herein particularly for intravenous administration may be utilized, where applicable or desirable, in a dosage form, composition, formulation or device for administration by any of the other routes herein contemplated or commonly employed. For example, a dose or doses could be given parenterally using a dosage form suitable for parenteral administration which may incorporate features or compositions described in respect of dosage forms suitable for oral administration, or be delivered in a sustained dosage form, such as a modified release, extended release, delayed release, slow release or repeat action dosage form.

Preferably the gap junction channel and/or hemichannel blocker or inhibitor compounds of the invention are combined with a pharmaceutically acceptable carrier or diluent to produce a pharmaceutical composition. Suitable carriers and diluents include isotonic saline solutions, for example phosphate-buffered saline. Suitable diluents and excipients also include, for example, water, saline, dextrose, glycerol, or the like, and combinations thereof. In addition, if desired substances such as wetting or emulsifying agents, stabilizing or pH buffering agents may also be present.

The term “pharmaceutically acceptable carrier” refers to any useful carriers, excipients, or stabilizers which are nontoxic to the cell or mammal being exposed thereto at the dosages and concentrations employed, and include pharmaceutical carriers that do not induce the production of antibodies harmful to the individual receiving the composition. Suitable carriers can be large, slowly metabolized macromolecules such as proteins, polysaccharides, polylactic acids, polyglycolic acids, polymeric amino acids, and amino acid copolymers. Often the physiologically acceptable carrier is an aqueous pH buffered solution. Other examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, polyethylene glycol (PEG), and Pluronics.

Pharmaceutically acceptable salts can also be present, e.g., mineral acid salts such as hydrochlorides, hydrobromides, phosphates, sulfates, and the like; and the salts of organic acids such as acetates, propionates, malonates, benzoates, and the like.

Suitable carrier materials include any carrier or vehicle commonly used as a base for creams, lotions, gels, emulsions, or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

An auxiliary agent such as casein, gelatin, albumin, glue, sodium alginate, carboxymethylcellulose, methylcellulose, hydroxyethylcellulose or polyvinyl alcohol may also be included in the formulation of the invention.

The dosage forms, formulations, devices and/or compositions of the invention may be formulated to optimize bioavailability and to maintain plasma concentrations within the therapeutic range, including for extended periods. Sustained delivery preparations, e.g., controlled delivery preparations, also optimize the drug concentration at the site of action and minimize periods of under and over medication, for example.

The dosage forms, devices and/or compositions useful in the invention may be provided for periodic administration, including once daily administration, for low dose controlled and/or low dose long-lasting in vivo release of a gap junction channel and/or hemichannel blocker or inhibitor compound.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of a gap junction channel and/or hemichannel blocker or inhibitor compounds.

Examples of dosage forms suitable for transdermal administration include, but are not limited to, transdermal patches, transdermal bandages, and the like. Examples of dosage forms suitable for topical administration of the compounds and formulations useful in the invention are any lotion, stick, spray, ointment, paste, cream, gel, etc., whether applied directly to the skin or via an intermed.

Examples of dosage forms suitable for suppository administration of the compounds and formulations useful in the invention include any solid dosage form inserted into a bodily orifice particularly those inserted rectally, vaginally and urethrally.

Examples of dosage forms suitable for transmucosal delivery of the compounds and formulations useful in the invention include depositories solutions for enemas, pessaries, tampons, creams, gels, pastes, foams, nebulised solutions, powders and similar formulations containing in addition to the active ingredients such carriers as are known in the art to be appropriate.

Examples of dosage of forms suitable for injection of the compounds and formulations useful in the invention include delivery via bolus such as single or multiple administrations by intravenous injection, subcutaneous, subdermal, and intramuscular administration or oral administration.

Examples of dosage forms suitable for depot administration of the compounds and formulations useful in the invention include pellets or small cylinders of active agent or solid forms wherein the active agent is entrapped in a matrix of biodegradable polymers, microemulsions, liposomes or is microencapsulated.

Examples of infusion devices for compounds and formulations useful in the invention include infusion pumps containing one or more gap junction channel and/or hemichannel blocker or inhibitor compounds and/or pre-complexed gap junction channel and/or hemichannel blocker or inhibitor compounds, at a desired amount for a desired number of doses or steady state administration, and include implantable drug pumps.

Examples of implantable infusion devices for compounds and formulations useful in the invention include any solid form in which the active agent is encapsulated within or dispersed throughout a biodegradable polymer or synthetic, polymer such as silicone, silicone rubber, silastic or similar polymer.

Examples of dosage forms suitable for inhalation or insufflation of compounds and formulations useful in the invention include compositions comprising solutions and/or suspensions in pharmaceutically acceptable, aqueous, or organic solvents, or mixture thereof and/or powders.

Examples of dosage forms suitable for buccal administration of the compounds and formulations useful in the invention include lozenges, tablets and the like, compositions comprising solutions and/or suspensions in pharmaceutically acceptable, aqueous, or organic solvents, or mixtures thereof and/or powders.

Examples of dosage forms suitable for sublingual administration of the compounds and formulations useful in the invention include lozenges, tablets and the like, compositions comprising solutions and/or suspensions in pharmaceutically acceptable, aqueous, or organic solvents, or mixtures thereof and/or powders.

Examples of controlled drug formulations for delivery of the compounds and formulations useful in the invention are found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C., Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. Further guidance concerning the analysis of extended release dosage forms has been provided by the FDA. See Guidance for Industry. Extended release oral dosage forms: development, evaluation, and application of in vitro/in vivo correlations. Rockville, MD: Center for Drug Evaluation and Research, Food and Drug Administration (1997).

Further examples of dosage forms useful in the methods of the invention include, but are not limited to, modified-release (MR) dosage forms including delayed-release (DR) forms; prolonged-action (PA) forms; controlled-release (CR) forms; extended-release (ER) forms; timed-release (TR) forms; and long-acting (LA) forms. For the most part, these terms are used to describe orally administered dosage forms, however these terms may be applicable to any of the dosage forms, formulations, compositions and/or devices described herein. These formulations effect delayed total drug release for some time after drug administration, and/or drug release in small aliquots intermittently after administration, and/or drug release slowly at a controlled rate governed by the delivery system, and/or drug release at a constant rate that does not vary, and/or drug release for a significantly longer period than usual formulations.

Modified-release dosage forms of the invention include dosage forms having drug release features based on time, course, and/or location which are designed to accomplish therapeutic or convenience objectives not offered by conventional or immediate-release forms. See, for example, Bogner, R. H. U.S. Pharmacist 22 (Suppl.):3-12 (1997); Scale-up of oral extended-release drug delivery systems: part I, an overview, Pharmaceutical Manufacturing 2:23-27 (1985). Extended-release dosage forms of the invention include, for example, as defined by The United States Food and Drug Administration (FDA), a dosage form that allows a reduction in dosing frequency to that presented by a conventional dosage form, e.g., a solution or an immediate-release dosage form. See, for example, Bogner, R. H. (1997) supra. Repeat action dosage forms of the invention include, for example, forms that contain two single doses of medication, one for immediate release and the second for delayed release. Bi-layered tablets, for example, may be prepared with one layer of drug for immediate release with the second layer designed to release drug later as either a second dose or in an extended-release manner. Targeted-release dosage forms of the invention include, for example, formulations that facilitate drug release and which are directed towards isolating or concentrating a drug in a body region, tissue, or site for absorption or for drug action.

Also useful in the invention are coated beads, granules or microspheres containing one or more gap junction channel and/or hemichannel blocker or inhibitor compounds and/or pre-complexed gap junction channel and/or hemichannel blocker or inhibitor compounds, which may be used to achieve modified release of one or more gap junction channel and/or hemichannel blocker or inhibitor compounds and/or pre-complexed gap junction channel and/or hemichannel blocker or inhibitor compounds by incorporation of the drug into coated beads, granules, or microspheres. In such systems, the gap junction channel and/or hemichannel blocker or inhibitor compound and/or pre-complexed gap junction channel and/or hemichannel blocker or inhibitor compound is distributed onto beads, pellets, granules or other particulate systems. See Ansel, H. C., Allen, L. V. and Popovich, N. G., Pharmaceutical Dosage Forms and Drug Delivery Systems, 7th Ed., Lippincott 1999, p. 232.

Methods for manufacture of microspheres suitable for drug delivery have been described. See, for example, Arshady, R. Polymer Eng Sci 30:1746-1758 (1989); see also, Arshady, R., Polymer Eng Sci 30:905-914 (1990); see also: Arshady R., Polymer Eng Sci 30:915-924 (1990). Various coating systems are commercially available. E.g., Aquacoat™ [FMC Corporation, Philadelphia] and Surerelease™ [Colorcon]; Aquacoat aqueous polymeric dispersion. Philadelphia: FMC Corporation, 1991; Surerelease aqueous controlled release coating system. West Point, Pa.: Colorcon, 1990; Butler, J., et al., Pharm Tech 22:122-138 (1998); Yazici, E., et al., Pharmaceut Dev Technol 1:175-183 (1996).

Variation in the thickness of the coats and in the type of coating materials used affects the rate at which the body fluids are capable of penetrating the coating to dissolve the gap junction channel and/or hemichannel blocker or inhibitor compound. Generally, the thicker the coat, the more resistant to penetration and the more delayed will be gap junction channel and/or hemichannel blocker or inhibitor compound release and dissolution. See Madan, P. L. U.S. Pharmacist 15:39-50 (1990). This provides the different desired sustained or extended release rates and the targeting of the coated beads to the desired segments of the gastrointestinal tract. Examples of film-forming polymers which can be used in water-insoluble release-slowing intermediate layer(s) (to be applied to a pellet, spheroid or tablet core) include ethylcellulose, polyvinyl acetate, Eudragit® RS, Eudragit® RL, etc. (Each of Eudragit® RS and Eudragit® RL is an ammonio methacrylate copolymer. The release rate can be controlled not only by incorporating therein suitable water-soluble pore formers, such as lactose, mannitol, sorbitol, etc., but also by the thickness of the coating layer applied. Multi-tablets may be formulated which include small spheroid-shaped compressed mini-tablets that may have a diameter of between 3 to 4 mm and can be placed in a gelatin capsule shell to provide the desired pattern of gap junction channel and/or hemichannel blocker or inhibitor compound release. Each capsule may contain 8-10 minitablets, some uncoated for immediate release and others coated for extended release of the gap junction channel and/or hemichannel blocker or inhibitor compound.

A number of methods may be employed to generate modified-release dosage forms of one or more gap junction channel and/or hemichannel blocker or inhibitor compounds suitable for oral administration to humans and other mammals. Two basic mechanisms available to achieve modified release drug delivery include altered dissolution or diffusion of drugs and excipients. Within this context, for example, four processes may be employed, either simultaneously or consecutively. These are as follows: (i) hydration of the device (e.g., swelling of the matrix); (ii) diffusion of water into the device; (iii) controlled or delayed dissolution of the drug; and (iv) controlled or delayed diffusion of dissolved or solubilized drug out of the device.

In order to formulate a range of dosage values, cell culture assays and animal studies can be used. The dosage of such compounds preferably lies within the dose that is therapeutically effective for at least 50% of the population, and that exhibits little or no toxicity at this level.

The effective dosage of each of the gap junction channel and/or hemichannel blocker or inhibitor compounds employed in the methods and compositions of the invention may vary depending on a number of factors including the particular gap junction channel and/or hemichannel blocker or inhibitor compound(s) employed, the cardiovascular therapeutic combinational partner if present, the mode of administration, the frequency of administration, the condition being treated, the severity of the condition being treated, the route of administration, the needs of a patient sub-population to be treated or the needs of the individual patient which different needs can be due to age, sex, body weight, relevant medical condition specific to the patient.

A suitable dose may be from about 0.001 to about 1 or to about 10 mg/kg body weight such as about 0.01 to about 0.5 mg/kg body weight. A suitable dose may however be from about 0.001 to about 0.1 mg/kg body weight such as about 0.01 to about 0.05 mg/kg body weight. Doses from about 1 to 100, 100-200, 200-300, 300-400, and 400-500 miligrams are appropriate, as are doses of about 500-750 micrograms and about 750-1000 micrograms. Other useful doses include from about 300 to about 1000 picomoles per dose, and about 0.05 to about 0.2 nanomoles per dose. Still other doses are within the following claims.

For example, in certain embodiments, the gap junction channel and/or hemichannel blocker or inhibitor compounds composition may be administered at about 0.01 nanomolar (mM) or 0.05 nM to about 200 nM final concentration. Preferably, the gap junction channel and/or hemichannel blocker or inhibitor compound composition is administered at about 0.1 nM to about 150 nM final concentration, more preferably, the gap junction channel and/or hemichannel blocker or inhibitor compound composition is applied at about 1 nM to about 100 nM final concentration, and more preferably, the gap junction channel and/or hemichannel blocker or inhibitor compound composition is administered at about 10-20 nM to about 100-150 nM final concentration. Additionally, gap junction channel and/or hemichannel blocker or inhibitor compound dose amounts include, for example, about 0.1-1, 1-2, 2-3, 3-4, or 4-5 milligrams (mg), from about 5 to about 10 mg, from about 10 to about 15 mg, from about 15 to about 20 mg, from about 20 to about 30 mg, from about 30 to about 40 mg, from about 40 to about 50 mg, from about 50 to about 75 mg, from about 75 to about 100 mg, from about 100 mg to about 250 mg, and from 250 mg to about 500 mg. Dose amounts from 500 to about 1000 milligrams or more or also provided, as noted above. Other doses include doses ranging from at least about 100 nanograms, including, for example at least about 200 nanograms, 600 nanograms, 2000 nanograms, 6000 nanograms and at least about 10,000 nanograms or more. Dose concentrations include concentrations of at least about 0.1 moles per liter, including, for example, at least about 0.3, 1.0, 3.0 and 10.0 nMoles/L. Dose concentrations also include concentrations of 0.1 nMoles/L, 0.3 nMoles/L, 1.0 nMoles/L, 3.0 nMoles/L and 10.0 nMoles/L. These dose concentrations are equivalent to 0.1, 0.3, 1, 3, 11 μg/L and administrable weight doses of 0.4, 1.0, 4.0, 10 and 39 micrograms/kg (μg/kg). Also within the invention are other doses ranging from 0.1 to 5.0 μg/kg and 0.1 to 10.0 μg/kg. Additionally, doses of about 0.4, 1.0, 4.0, 10 and 39 μg/kg are within the invention. Doses of at least about 0.4, 1.0, 4.0, 10 and 39 μg/kg are also within the invention.

Still other dosage levels between about 1 nanogram (ng)/kg and about 1 mg/kg body weight per day of each of the agents described herein. In certain embodiments, the dosage of each of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 0.01 mg per kg body weight, about 0.01 mg to about 0.1 mg per kg body weight, or about 0.1 mg to about 1 mg per kg body weight. In certain embodiments, the dosage of each of the subject compounds will generally be in the range of about 0.001 mg to about 0.01 mg/kg body weight, about 0.01 mg to about 0.1 mg/kg body weight, about 0.1 mg to about 1 mg/kg body weight. If more than one gap junction channel and/or hemichannel blocker or inhibitor compound is used, the dosage of each gap junction channel and/or hemichannel blocker or inhibitor compound need not be in the same range as the other.

Conveniently, if infused, the gap junction channel and/or hemichannel blocker or inhibitor compound is administered for at least about 0.5 to 1 hour, at least about 1-2 hours, at least about 2-4 hours, at least about 4-6 hours, at least about 6-8 hours, at least about 8-10 hours, at least about 12 hours, or at least about 24 hours.

As noted herein, the doses of a gap junction channel and/or hemichannel blocker or inhibitor compounds, for example, administered in combination, or other therapeutic agents administered in combination with either or both, can be adjusted down from the doses administered when given alone.

The combined use of several agents may reduce the required dosage for any individual agent because the onset and duration of effect of the different agents may be complementary. In a preferred embodiment, the combined use of two or more gap junction channel and/or hemichannel blocker or inhibitor compounds has an additive, synergistic or super-additive effect.

In some cases, the combination of a gap junction channel and/or hemichannel blocker or inhibitor compound and another therapeutic agent, or other agents administered in combination with either or both, have an additive effect. In other cases, the combination can have greater-than-additive effect. Such an effect is referred to herein as a “supra-additive” effect, and may be due to synergistic or potentiated interaction.

In another preferred embodiment, the combined use of a gap junction channel and/or hemichannel blocker or inhibitor compound and another therapeutic agent, reduces the frequency in which said agent is administered compared to the frequency when said agent is administered alone. Thus, these combinations allow the use of lower and/or fewer doses of each agent than previously required to achieve desired therapeutic goals.

Doses may be administered in single or divided applications. The doses may be administered once, or application may be repeated. Typically, administration can be by infusion in addition to or instead of multiple single adminstrations.

One or more gap junction channel and/or hemichannel blocker or inhibitor compounds and another cardiovascular therapeutic agent, if desired, may be administered by the same or different routes. The various agents of the invention can be administered separately at different times during the course of therapy, or concurrently in divided or single combination forms.

In one aspect of the invention a gap junction channel and/or hemichannel blocker or inhibitor compound is administered in one composition and another cardiovascular therapeutic agent is administered in a second composition. In one embodiment the first composition comprising a gap junction channel and/or hemichannel blocker or inhibitor compound is administered before the second composition comprising another therapeutic agent. In one embodiment the first composition comprising a gap junction channel and/or hemichannel blocker or inhibitor compound is administered after the second composition comprising another cardiovascular therapeutic agent. In one preferred embodiment the first composition comprising a gap junction channel and/or hemichannel blocker or inhibitor compound is administered before and after the second composition comprising another therapeutic agent. In one embodiment the second composition comprising another therapeutic agent is administered before and after the first composition comprising the gap junction channel and/or hemichannel blocker or inhibitor compound agent. In one embodiment the first composition comprising a gap junction channel and/or hemichannel blocker or inhibitor compound is administered about the same time as the second composition comprising another therapeutic agent.

The delivery of a formulation comprising a gap junction channel and/or hemichannel blocker or inhibitor compound, alone or together with another therapeutic agent, over a period of time, in some instances for about 1-2 hours, about 2-4 hours, about 4-6 hours, about 6-8, or about 24 hours or longer, may also be accomplished using slow release or depot formulations, for example, as well as transdermal formulations and devices.

Strategies to improve the oral bioavailability of proteins and peptides have ranged from changing their physicochemical properties by modification of their lipophilicity and enzyme susceptibility, to adding novel functionality using transport-carrier molecules that are recognized by endogenous transport-carrier systems in the gastrointestinal tract and/or to their inclusion in specially adapted drug carrier systems. Marketed polymeric-based systems have attracted considerable attention in the controlled release in targeting particular organs/tissues, and in their ability to deliver proteins and peptides. They can effectively deliver the proteins to a target site and thus increase the therapeutic benefit, while minimizing side effects. Protein association with polymer-based carriers, such as polymeric microparticles, nanoparticles, hydrogels or patches is a useful approach to improve oral protein bioavailability. Polymer-based carriers can protect proteins from the gastrointestinal environment and allow the modulation of physicochemical and protein release properties and consequently the biological behavior. Also, from the perspective of improving oral absorption, the major effect of carriers is to increase epithelial membrane permeability, thereby leading to higher bioavailability.

Dosage forms of the compounds and formulations of the invention, extended gap junction channel and/or hemichannel blocker or inhibitor compound action may be achieved by affecting the rate at which the gap junction channel and/or hemichannel blocker or inhibitor compound is released from the dosage form and/or by slowing the transit time of the dosage form through the gastrointestinal tract (see Bogner, R. H., US Pharmacist 22 (Suppl.):3-12 (1997)). The rate of drug release from solid dosage forms may be modified by the technologies described below which, in general, are based on the following: 1) modifying drug dissolution by controlling access of biologic fluids to the drug through the use of barrier coatings; 2) controlling drug diffusion rates from dosage forms; and 3) chemically reacting or interacting between the drug substance or its pharmaceutical barrier and site-specific biological fluids. Systems by which these objectives are achieved are also provided herein. In one approach, employing digestion as the release mechanism, the gap junction channel and/or hemichannel blocker or inhibitor compound is either coated or entrapped in a substance that is slowly digested or dispersed into the intestinal tract. The rate of availability of the gap junction channel and/or hemichannel blocker or inhibitor compound is a function of the rate of digestion of the dispersible material. Therefore, the release rate, and thus the effectiveness of the gap junction channel and/or hemichannel blocker or inhibitor compound varies from subject to subject depending upon the ability of the subject to digest the material.

A further form of slow release dosage form of the compounds and formulations of the invention is any suitable osmotic system where semi-permeable membranes of for example cellulose acetate, cellulose acetate butyrate, cellulose acetate propionate, is used to control the release of gap junction channel and/or hemichannel blocker or inhibitor compound. These can be coated with aqueous dispersions of enteric lacquers without changing release rate. An example of such an osmotic system is an osmotic pump device, such as the Oros™ device developed by Alza Inc. (U.S.A.).

Other devices useful in the methods of the invention utilize monolithic matrices including, for example, slowly eroding or hydrophilic polymer matrices, in which one or more gap junction channel and/or hemichannel blocker or inhibitor compounds are compressed or embedded.

Monolithic matrix devices comprising compounds and formulations useful in the invention include those formed using, for example, gap junction channel and/or hemichannel blocker or inhibitor compounds dispersed in a soluble matrix, which become increasingly available as the matrix dissolves or swells; examples include hydrophilic colloid matrices, such as hydroxypropylcellulose (BP) or hydroxypropyl cellulose (USP); hydroxypropyl methylcellulose (HPMC; BP, USP); methylcellulose (MC; BP, USP); calcium carboxymethylcellulose (Calcium CMC; BP, USP); acrylic acid polymer or carboxy polymethylene (Carbopol) or Carbomer (BP, USP); or linear glycuronan polymers such as alginic acid (BP, USP), for example those formulated into microparticles from alginic acid (alginate)-gelatin hydrocolloid coacervate systems, or those in which liposomes have been encapsulated by coatings of alginic acid with poly-L-lysine membranes. Gap junction channel and/or hemichannel blocker or inhibitor compound release occurs as the polymer swells, forming a matrix layer that controls the diffusion of aqueous fluid into the core and thus the rate of diffusion of gap junction channel and/or hemichannel blocker or inhibitor compound from the system.

In such systems, the rate of gap junction channel and/or hemichannel blocker or inhibitor compound release depends upon the tortuous nature of the channels within the gel, and the viscosity of the entrapped fluid, such that different release kinetics can be achieved, for example, zero-order, or first-order combined with pulsatile release. Where such gels are not cross-linked, there is a weaker, non-permanent association between the polymer chains, which relies on secondary bonding. With such devices, high loading of the gap junction channel and/or hemichannel blocker or inhibitor compound is achievable, and effective blending is frequent. Devices may contain 20-80% of gap junction channel and/or hemichannel blocker or inhibitor compound (w/w), along with gel modifiers that can enhance gap junction channel and/or hemichannel blocker or inhibitor compound diffusion; examples of such modifiers include sugars that can enhance the rate of hydration, ions that can influence the content of cross-links, and pH buffers that affect the level of polymer ionization. Hydrophilic matrix devices may also contain one or more pH buffers, surfactants, counter-ions, lubricants such as magnesium stearate (BP, USP) and a glidant such as colloidal silicon dioxide (USP; colloidal anhydrous silica, BP) in addition to gap junction channel and/or hemichannel blocker or inhibitor compound and hydrophilic matrix.

Monolithic matrix devices comprising compounds and formulations useful in the invention also include those formed using, for example, gap junction channel and/or hemichannel blocker or inhibitor compound particles are dissolved in an insoluble matrix, from which Gap junction channel and/or hemichannel blocker or inhibitor compound becomes available as solvent enters the matrix, often through channels, and dissolves the Gap junction channel and/or hemichannel blocker or inhibitor compound particles. Examples include systems formed with a lipid matrix, or insoluble polymer matrix, including preparations formed from Carnauba wax (BP; USP); medium-chain triglyceride such as fractionated coconut oil (BP) or triglycerida saturata media (PhEur); or cellulose ethyl ether or ethylcellulose (BP, USP). Lipid matrices are simple and easy to manufacture, and incorporate the following blend of powdered components: lipids (20-40% hydrophobic solids w/w) which remain intact during the release process; Gap junction channel and/or hemichannel blocker or inhibitor compound, e.g., channeling agent, such as sodium chloride or sugars, which leaches from the formulation, forming aqueous micro-channels (capillaries) through which solvent enters, and through which Gap junction channel and/or hemichannel blocker or inhibitor compound is released. In this system, the Gap junction channel and/or hemichannel blocker or inhibitor compound is embedded in an inert insoluble polymer and is released by leaching of aqueous fluid, which diffuses into the core of the device through capillaries formed between particles, and from which the Gap junction channel and/or hemichannel blocker or inhibitor compound diffuses out of the device. The rate of release is controlled by the degree of compression, particle size, and the nature and relative content (w/w) of excipients. An example of such a device is that of Ferrous Gradumet (Martindale 33rd Ed., 1360.3). A further example of a suitable insoluble matrix is an inert plastic matrix. By this method, Gap junction channel and/or hemichannel blocker or inhibitor compounds are granulated with an inert plastic material such as polyethylene, polyvinyl acetate, or polymethacrylate, and the granulated mixture is then compressed into tablets. Once ingested, the Gap junction channel and/or hemichannel blocker or inhibitor compound is slowly released from the inert plastic matrix by diffusion. See, for example, Bodmeier, R. & Paeratakul, O., J Pharm Sci 79:32-26 (1990); Laghoueg, N., et al., Int J Pharm 50:133-139 (1989); Buckton, G., et al., Int J Pharm 74:153-158 (1991). The compression of the tablet creates the matrix or plastic form that retains its shape during the leaching of the Gap junction channel and/or hemichannel blocker or inhibitor compound and through its passage through the gastrointestinal tract. An immediate-release portion of Gap junction channel and/or hemichannel blocker or inhibitor compound may be compressed onto the surface of the tablet. The inert tablet matrix, expended of Gap junction channel and/or hemichannel blocker or inhibitor compound, is excreted with the feces. An example of a successful dosage form of this type is Gradumet (Abbott; see, for example, Ferro-Gradumet, Martindale 33rd Ed., p. 1860.4).

Further examples of monolithic matrix devices useful in the methods of the invention include compositions and formulations of the invention incorporated in pendent attachments to a polymer matrix. See, for example, Scholsky, K. M. and Fitch, R. M., J Controlled Release 3:87-108 (1986). In these devices, Gap junction channel and/or hemichannel blocker or inhibitor compounds may be attached by means of an ester linkage to poly(acrylate) ester latex particles prepared by aqueous emulsion polymerization. Still further examples of monolithic matrix devices of the invention incorporate dosage forms in which the Gap junction channel and/or hemichannel blocker or inhibitor compound is bound to a biocompatible polymer by a labile chemical bond, e.g., polyanhydrides prepared from a substituted anhydride (itself prepared by reacting an acid chloride with the drug: methacryloyl chloride and the sodium salt of methoxy benzoic acid) have been used to form a matrix with a second polymer (Eudragit RL) which releases drug on hydrolysis in gastric fluid. See Chafi, N., et al., Int J Pharm 67:265-274 (1992).

Modified release forms of one or more Gap junction channel and/or hemichannel blocker or inhibitor compounds may also be prepared by microencapsulation. Microencapsulation is a process by which solids, liquids, or even gasses may be encapsulated into microscopic size particles through the formation of thin coatings of “wall” material around the substance being encapsulated such as disclosed in U.S. Pat. Nos. 3,488,418; 3,391,416 and 3,155,590. Gelatin (BP, USP) is commonly employed as a wall-forming material in microencapsulated preparations, but synthetic polymers such as polyvinyl alcohol (USP), ethylcellulose (BP, USP), polyvinyl chloride, and other materials may also be used. See, for example, Zentner, G. M., et al., J Controlled Release 2:217-229 (1985); Fites, A. L., et al., J Pharm Sci 59:610-613 (1970); Samuelov, Y., et al., J Pharm Sci 68:325-329 (1979). Different rates of Gap junction channel and/or hemichannel blocker or inhibitor compound release may be obtained by changing the core-to-wall ratio, the polymer used for the coating, or the method of microencapsulation. See, for example: Yazici, E., Oner, et al., Pharmaceut Dev Technol; 1:175-183 (1996).

Other useful approaches include those in which the Gap junction channel and/or hemichannel blocker or inhibitor compound is incorporated into polymeric colloidal particles or microencapsulates (microparticles, microspheres or nanoparticles) in the form or reservoir and matrix devices. See: Douglas, S. J., et al., C.R. C. Crit Rev Therap Drug Carrier Syst 3:233-261 (1987); Oppenheim, R. C., Int J Pharm 8:217-234 (1981); Higuchi, T., J Pharm Sci 52:1145-1149 (1963).

Formulations of drugs suitable for transdermal delivery are known to those skilled in the art, and are described in references such as Ansel et al., (supra). Methods known to enhance the delivery of drugs by the percutaneous route include chemical skin penetration enhancers, which increase skin permeability by reversibly damaging or otherwise altering the physicochemical nature of the stratum corneum to decrease its resistance to drug diffusion. See Shah, V., Peck, C. C., and Williams, R. L., Skin penetration enhancement: clinical pharmacological and regulatory considerations, In: Walters, K. A. and Hadgraft, J. (Eds.) Pharmaceutical skin penetration enhancement. New York: Dekker, (1993). Skin penetration enhancers suitable for formulation with Gap junction channel and/or hemichannel blocker or inhibitor compounds in transdermal drug delivery systems may be chosen from the following list: acetone, laurocapram, dimethylacetamide, dimethylformamide, dimethylsulphoxide, ethanol, oleic acid, polyethylene glycol, propylene glycol and sodium lauryl sulphate. Further skin penetration enhancers may be found in publications known to those skilled in the art. See, for example, Osborne, D. W., & Henke, J. J., Pharm Tech 21:50-66 (1997); Rolf, D., “Pharm Tech 12:130-139 (1988). In addition to chemical means, there are physical methods that enhance transdermal drug delivery and penetration of the compounds and formulations of the invention. These include iontophoresis and sonophoresis. Formulations suitable for administration by iontophoresis or sonophoresis may be in the form of gels, creams, or lotions.

Transdermal delivery, methods or formulations of the invention, may utilize, among others, monolithic delivery systems, drug-impregnated adhesive delivery systems (e.g., the Latitude™ drug-in-adhesive system from 3M), active transport devices and membrane-controlled systems. Transdermal delivery dosage forms of the invention include those which substitute the Gap junction channel and/or hemichannel blocker or inhibitor compound, for the diclofenic or other pharmaceutically acceptable salt thereof referred to in the transdermal delivery systems disclosed in, by way of example, U.S. Pat. Nos. 6,193,996, and 6,262,121.

Other dosage forms include variants of the oral dosage forms adapted for suppository or other parenteral use. When rectally administered in the form of suppositories, for example, these compositions may be prepared by mixing one or more compounds and formulations of the invention with a suitable non-irritating excipient, such as cocoa butter, synthetic glyceride esters or polyethylene glycols, which are solid at ordinary temperatures, but liquify and/or dissolve in the rectal cavity to release the Gap junction channel and/or hemichannel blocker or inhibitor compound. Suppositories are generally solid dosage forms intended for insertion into body orifices including rectal, vaginal and occasionally urethrally and can be long acting or slow release. Suppositories include a base that can include, but is not limited to, materials such as alginic acid, which will prolong the release of the pharmaceutically acceptable active ingredient over several hours (5-7).

Transmucosal administration of the compounds and formulations useful in the invention may utilize any mucosal membrane but commonly utilizes the nasal, buccal, vaginal and rectal tissues. Formulations suitable for nasal administration of the compounds and formulations of the invention may be administered in a liquid form, for example, nasal spray, nasal drops, or by aerosol administration by nebulizer, including aqueous or oily solutions of the Gap junction channel and/or hemichannel blocker or inhibitor compound. Formulations for nasal administration, wherein the carrier is a solid, include a coarse powder having a particle size, for example, of less than about 100 microns, preferably less, most preferably one or two times per day than about 50 microns, which is administered in the manner in which snuff is taken, i.e., by rapid inhalation through the nasal passage from a container of the powder held close up to the nose. Compositions in solution may be nebulized by the use of inert gases and such nebulized solutions may be breathed directly from the nebulizing device or the nebulizing device may be attached to a facemask, tent or intermittent Gap junction channel and/or hemichannel blocker or inhibitor compounds may be administered orally or nasally from devices that deliver the formulation in an appropriate manner. Formulations may be prepared as aqueous solutions for example in saline, solutions employing benzyl alcohol or other suitable preservatives, absorption promoters to enhance bio-availability, fluorocarbons, and/or other solubilising or dispersing agents known in the art.

Compositions may be prepared according to conventional methods by dissolving or suspending an amount of a Gap junction channel and/or hemichannel blocker or inhibitor compound(s) (s) ingredient in a diluent. The amount of Gap junction channel and/or hemichannel blocker or inhibitor compound is from between 0.1 mg to 1000 mg per ml of diluent. In some embodiments, dosage forms of 100 mg and 200 mg of a Gap junction channel and/or hemichannel blocker or inhibitor compound are provided. By way of example only, the amount of Gap junction channel and/or hemichannel blocker or inhibitor compound may range from about 1 mg to about 750 mg or more (for example, about 1 mg, about 10 mg, about 25 mg, about 50 mg, about 100 mg, about 150 mg, about 200 mg, about 250 mg, about 400 mg, about 500 mg, about 600 mg, about 750 mg, about 800 mg, about 1000 mg, and about 1200 mg). Other doses include doses ranging from at least about 100 nanograms, including, for example at least about 200 nanograms, 600 nanograms, 2000 nanograms, 6000 nanograms and at least about 10,000 nanograms or more. Dose concentrations include concentrations of at least about 0.1 moles per liter, including, for example, at least about 0.3, 1.0, 3.0 and 10.0 nMoles/L. Dose concentrations also include concentrations of 0.1 nMoles/L, 0.3 nMoles/L, 1.0 nMoles/L, 3.0 nMoles/L and 10.0 nMoles/L. These dose concentrations are equivalent to 0.1, 0.3, 1, 3, 11 μg/L and administrable weight doses of 0.4, 1.0, 4.0, 10 and 39 micrograms/kg (μg/kg). Also within the invention are other doses ranging from 0.1 to 5.0 μg/kg and 0.1 to 10.0 μg/kg. Additionally, doses of about 0.4, 1.0, 4.0, 10 and 39 μg/kg are within the invention. Doses of at least about 0.4, 1.0, 4.0, 10 and 39 μg/kg are also within the invention. Other amounts within these ranges may also be used and are specifically contemplated though each number in between is not expressly set out.

Gap junction channel and/or hemichannel blocker or inhibitor compounds can be provided and administered in forms suitable for once-a-day dosing. An acetate, phosphate, citrate or glutamate buffer may be added allowing a pH of the final composition to be from about 5.0 to about 9.5; optionally a carbohydrate or polyhydric alcohol tonicifier and, a preservative selected from the group consisting of m-cresol, benzyl alcohol, methyl, ethyl, propyl and butyl parabens and phenol may also be added. Water for injection, tonicifying agents such as sodium chloride, as well as other excipients, may also be present, if desired. For parenteral administration, formulations are isotonic or substantially isotonic to avoid irritation and pain at the site of administration.

The terms buffer, buffer solution and buffered solution, when used with reference to hydrogen-ion concentration or pH, refer to the ability of a system, particularly an aqueous solution, to resist a change of pH on adding acid or alkali, or on dilution with a solvent. Characteristic of buffered solutions, which undergo small changes of pH on addition of acid or base, is the presence either of a weak acid and a salt of the weak acid, or a weak base and a salt of the weak base. An example of the former system is acetic acid and sodium acetate. The change of pH is slight as long as the amount of hydroxyl ion added does not exceed the capacity of the buffer system to neutralize it.

Maintaining the pH of the formulation in the range of approximately 5.0 to about 9.5 can enhance the stability of the parenteral formulation of the present invention. Other pH ranges, for example, include, about 5.5 to about 9.0, or about 6.0 to about 8.5, or about 6.5 to about 8.0, or, preferably, about 7.0 to about 7.5.

The buffer used may be selected from any of the following, for example, an acetate buffer, a phosphate buffer or glutamate buffer, the most preferred buffer being a phosphate buffer. Carriers or excipients can also be used to facilitate administration of the compositions and formulations of the invention. Examples of carriers and excipients include calcium carbonate, calcium phosphate, various sugars such as lactose, glucose, or sucrose, or types of starch, cellulose derivatives, gelatin, polyethylene glycols and physiologically compatible solvents. A stabilizer may be included, but will generally not be needed. If included, however, an example of a stabilizer useful in the practice of the invention is a carbohydrate or a polyhydric alcohol. The polyhydric alcohols include such compounds as sorbitol, mannitol, glycerol, xylitol, and polypropylene/ethylene glycol copolymer, as well as various polyethylene glycols (PEG) of molecular weight 200, 400, 1450, 3350, 4000, 6000, and 8000). The carbohydrates include, for example, mannose, ribose, trehalose, maltose, inositol, lactose, galactose, arabinose, or lactose.

Isotonicity agents, or agents to maintain isotonicity, may also be used or included.

The United States Pharmacopeia (USP) states that anti-microbial agents in bacteriostatic or fungistatic concentrations must be added to preparations contained in multiple dose containers. They must be present in adequate concentration at the time of use to prevent the multiplication of microorganisms inadvertently introduced into the preparation while withdrawing a portion of the contents with a hypodermic needle and syringe, or using other invasive means for delivery, such as pen injectors. Antimicrobial agents should be evaluated to ensure compatibility with all other components of the formula, and their activity should be evaluated in the total formula to ensure that a particular agent that is effective in one formulation is not ineffective in another. It is not uncommon to find that a particular agent will be effective in one formulation but not effective in another formulation. While the preservative for use in the practice of the invention can range from 0.005 to 1.0% (w/v), the preferred range for each preservative, alone or in combination with others, is: benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens are lower alkyl esters of para-hydroxybenzoic acid. A detailed description of each preservative is set forth in “Remington's Pharmaceutical Sciences” as well as Pharmaceutical Dosage Forms: Parenteral Medications, Vol. 1, 1992, Avis et al. For these purposes, the Gap junction channel and/or hemichannel blocker or inhibitor compound may be administered parenterally (including subcutaneous injections, intravenous, intramuscular, intradermal injection or infusion techniques) or by inhalation spray in dosage unit formulations containing conventional non-toxic pharmaceutically acceptable carriers, adjuvants and vehicles.

If desired, the parenteral formulation may be thickened with a thickening agent such as a methylcellulose. The formulation may be prepared in an emulsified form, either water in oil or oil in water. Any of a wide variety of pharmaceutically acceptable emulsifying agents may be employed including, for example, acacia powder, a non-ionic surfactant or an ionic surfactant. It may also be desirable to add suitable dispersing or suspending agents to the pharmaceutical formulation. These may include, for example, aqueous suspensions such as synthetic and natural gums, e.g., tragacanth, acacia, alginate, dextran, sodium carboxymethylcellulose, methylcellulose, polyvinyl-pyrrolidone or gelatin.

It is possible that other ingredients may be present in a parenteral pharmaceutical formulation useful the invention. Such additional ingredients may include wetting agents, oils (e.g., a vegetable oil such as sesame, peanut or olive), analgesic agents, emulsifiers, antioxidants, bulking agents, tonicity modifiers, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention. Regarding pharmaceutical formulations, see also, Pharmaceutical Dosage Forms: Parenteral Medications, Vol. 1, 2nd ed., Avis et al., Eds., Mercel Dekker, New York, N.Y. 1992.

Suitable routes of parenteral administration include intramuscular, intravenous, subcutaneous, intraperitoneal, subdermal, intradermal, intraarticular, intrathecal and the like. Mucosal delivery is also permissible. The dose and dosage regimen will depend upon the weight and health of the subject.

In addition to the above means of achieving extended drug action, the rate and duration of Gap junction channel and/or hemichannel blocker or inhibitor compound delivery may be controlled by, for example by using mechanically controlled drug infusion pumps.

The Gap junction channel and/or hemichannel blocker or inhibitor compound(s) can be administered in the form of a depot injection that may be formulated in such a manner as to permit a sustained release of the Gap junction channel and/or hemichannel blocker or inhibitor compound. The Gap junction channel and/or hemichannel blocker or inhibitor compound can be compressed into pellets or small cylinders and implanted subcutaneously or intramuscularly. The pellets or cylinders may additionally be coated with a suitable biodegradable polymer chosen so as to provide a desired release profile. The Gap junction channel and/or hemichannel blocker or inhibitor compound may alternatively be micropelleted. The Gap junction channel and/or hemichannel blocker or inhibitor compound micropellets using bioacceptable polymers can be designed to allow release rates to be manipulated to provide a desired release profile. Alternatively, injectable depot forms can be made by forming microencapsulated matrices of the Gap junction channel and/or hemichannel blocker or inhibitor compound in biodegradable polymers such as polylactide-polyglycolide. Depending on the ratio of Gap junction channel and/or hemichannel blocker or inhibitor compound to polymer, and the nature of the particular polymer employed, the rate of Gap junction channel and/or hemichannel blocker or inhibitor compound release can be controlled. Depot injectable formulations can also be prepared by entrapping the Gap junction channel and/or hemichannel blocker or inhibitor compound in liposomes, examples of which include unilamellar vesicles, large unilamellar vesicles and multilamellar vesicles. Liposomes can be formed from a variety of phospholipids, such as cholesterol, stearyl amine or phosphatidylcholines. Depot injectable formulations can also be prepared by entrapping the Gap junction channel and/or hemichannel blocker or inhibitor compound in microemulsions that are compatible with body tissue. By way of example, reference is made to U.S. Pat. Nos. 6,410,041 and 6,362,190.

Implantable infusion devices may employ inert material such as biodegradable polymers listed above or synthetic silicones, for example, cylastic, silicone rubber or other polymers manufactured by the Dow-Corning Corporation. The polymer may be loaded with Gap junction channel and/or hemichannel blocker or inhibitor compound and any excipients. Implantable infusion devices may also comprise a coating of, or a portion of, a medical device wherein the coating comprises the polymer loaded with Gap junction channel and/or hemichannel blocker or inhibitor compound and any excipient. Such an implantable infusion device may be prepared as disclosed in U.S. Pat. No. 6,309,380 by coating the device with an in vivo biocompatible and biodegradable or bioabsorbable or bioerodibleerodible liquid or gel solution containing a polymer with the solution comprising a desired dosage amount of Gap junction channel and/or hemichannel blocker or inhibitor compound and any excipients. The solution is converted to a film adhering to the medical device thereby forming the implantable Gap junction channel and/or hemichannel blocker or inhibitor compound-deliverable medical device. An implantable infusion device may also be prepared by the in situ formation of a Gap junction channel and/or hemichannel blocker or inhibitor compound containing solid matrix as disclosed in U.S. Pat. No. 6,120,789. Implantable infusion devices may be passive or active, as known in the art.

Also useful in methods of the invention are microemulsions, i.e., such as fluid and stable homogeneous solutions composed of a hydrophilic phase, a lipophilic phase, at least one surfactant (SA) and at least one cosurfactant (CoSA). Examples of suitable surfactants include mono-, di- and triglycerides and polyethylene glycol (PEG) mono- and diesters. A cosurfactant, also sometimes known as “co-surface-active agentm,” is a chemical compound having hydrophobic character, intended to cause the mutual solubilization of the aqueous and oily phases in a microemulsion. Examples of suitable co-surfactants include ethyl diglycol, lauric esters of propylene glycol, oleic esters of polyglycerol, and related compounds.

Gap junction channel and/or hemichannel blocker or inhibitor compounds may also be delivered using various polymers to enhance bioavailability by increasing adhesion to mucosal surfaces, by decreasing the rate of degradation by hydrolysis or enzymatic degradation of the Gap junction channel and/or hemichannel blocker or inhibitor compound, and by increasing the surface area of the Gap junction channel and/or hemichannel blocker or inhibitor compound relative to the size of the particle. Suitable polymers can be natural or synthetic, and can be biodegradable or non-biodegradable. Delivery of low molecular weight active agents, such as for example Gap junction channel and/or hemichannel blocker or inhibitor compounds, may occur by either diffusion or degredation of the polymeric system. Representative natural polymers include proteins such as zein, modified zein, casein, gelatin, gluten, serum albumin, and collagen, polysaccharides such as cellulose, dextrans, and polyhyaluronic acid. Synthetic polymers are generally preferred due to the better characterization of degradation and release profiles. Representative synthetic polymers include polyphosphazenes, poly(vinyl alcohols), polyamides, polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate). Synthetically modified natural polymers include cellulose derivatives such as alkyl celluloses, hydroxyalkyl celluloses, cellulose ethers, cellulose esters, and nitrocelluloses. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. Each of the polymers described above can be obtained from commercial sources such as Sigma Chemical Co., St. Louis, Mo., Polysciences, Warrenton, Pa., Aldrich Chemical Co., Milwaukee, Wis., Fluka, Ronkonkoma, N.Y., and BioRad, Richmond, Calif. or can be synthesized from monomers obtained from these suppliers using standard techniques.

The polymers described above can be separately characterized as biodegradable, non-biodegradable, and bioadhesive polymers. Representative synthetic degradable polymers include polyhydroxy acids such as polylactides, polyglycolides and copolymers thereof, poly(ethylene terephthalate), poly(butic acid), poly(valeric acid), poly(lactide-co-caprolactone), polyanhydrides, polyorthoesters and blends and copolymers thereof. Representative natural biodegradable polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers. Examples of non-biodegradable polymers include ethylene vinyl acetate, poly(meth)acrylic acid, polyamides, polyethylene, polypropylene, polystyrene, polyvinyl chloride, polyvinylphenol, and copolymers and mixtures thereof. Hydrophilic polymers and hydrogels tend to have bioadhesive properties. Hydrophilic polymers that contain carboxylic groups (e.g., poly[acrylic acid]) tend to exhibit the best bioadhesive properties. Polymers with the highest concentrations of carboxylic groups are preferred when bioadhesiveness on soft tissues is desired. Various cellulose derivatives, such as sodium alginate, carboxymethylcellulose, hydroxymethylcellulose and methylcellulose also have bioadhesive properties. Some of these bioadhesive materials are water-soluble, while others are hydrogels. Polymers such as hydroxypropylmethylcellulose acetate succinate (HPMCAS), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP) may be utilized to enhance the bioavailability of Gap junction channel and/or hemichannel blocker or inhibitor compounds with which they are complexed. Rapidly bioerodible polymers such as poly(lactide-co-glycolide), polyanhydrides, and polyorthoesters, whose carboxylic groups are exposed on the external surface as their smooth surface erodes, can also be used for bioadhesive Gap junction channel and/or hemichannel blocker or inhibitor compound delivery systems. In addition, polymers containing labile bonds, such as polyanhydrides and polyesters, are well known for their hydrolytic reactivity. Their hydrolytic degradation rates can generally be altered by simple changes in the polymer backbone. Upon degradation, these materials also expose carboxylic groups on their external surface, and can also be used as B natriuretic signal peptide fragment agent delivery systems.

Other agents that may enhance bioavailability or absorption of one or more Gap junction channel and/or hemichannel blocker or inhibitor compounds can act by facilitating or inhibiting transport across the intestinal mucosa. For example, agents that increase blood flow, such as vasodilators, may increase the rate of absorption of orally administered Gap junction channel and/or hemichannel blocker or inhibitor compound by increasing the blood flow to the gastrointestinal tract. Vasodilators constitute another class of agents that may enhance the bioavailability of Gap junction channel and/or hemichannel blocker or inhibitor compounds.

Other mechanisms of enhancing bioavailability of the compositions and formulations useful in the invention include the inhibition of reverse active transport mechanisms. For example, it is now thought that one of the active transport mechanisms present in the intestinal epithelial cells is p-glycoprotein transport mechanism which facilitates the reverse transport of substances, which have diffused or have been transported inside the epithelial cell, back into the lumen of the intestine. Inhibition of this p-glycoprotein mediated active transport system will cause less drug to be transported back into the lumen and will thus increase the net drug transport across the gut epithelium and will increase the amount of drug ultimately available in the blood. Various p-glycoprotein inhibitors are well known and appreciated in the art. These include, water soluble vitamin E; polyethylene glycol; poloxamers including Pluronic F-68; Polyethylene oxide; polyoxyethylene castor oil derivatives including Cremophor EL and Cremophor RH 40; Chrysin, (+)-Taxifolin; Naringenin; Diosmin; Quercetin; and the like.

Thus, while the delivery period will be dependent upon both the condition and the agent and the therapeutic effect which is desired, continuous or slow-release delivery for about 0.5-1 hour, about 1-2 hours, about 2-4 hours, about 4-6 hours, about 6-8, or about 24 hours or longer is provided. In accordance with the present invention, this is achieved by inclusion of a Gap junction channel and/or hemichannel blocker or inhibitor compound, alone or toether with another cardiovascular therapeutic agent, in a formulation together with a pharmaceutically acceptable carrier or vehicle, particularly in the form of a formulation for continuous or slow-release administration.

As noted, the one or more agents of the invention may be administered before, during, immediately following a procedure in or on a subject, for example an angioplasty procedure or other physical intervention, such as stenting. They are preferably administered, for example, before and/or during a procedure or within about 24, about 12, about 10, about 9, about 8, about 7, about 6, about 5, about 4, about 3, about 2 hours or within about 60, about 45, about 30, about 15, about 10, about 5, about 4, about 3, about 2, about 1 minute following a procedure, for example.

The routes of administration and dosages described herein are intended only as a guide since a skilled physician will consider the optimum route of administration and dosage for any particular patient and condition.

Any of the methods of treating a subject having or at risk for a cardiovascular disorder may utilize the administration of any of the doses, dosage forms, formulations, and/or compositions herein described.

### Pharmaceutical Compositions

The present invention is directed to pharmaceutical compositions and their methods of use for preventing and/or treating a cardiovascular disorder wherein the composition comprises a therapeutically effective amount of a Gap junction channel and/or hemichannel blocker or inhibitor compound, alone or together with another cardiovascular therapeutic agent.

Accordingly, in one aspect, the invention provides compositions for use in preventing and/or treating a cardiovascular disorder, which comprises or consists essentially of at least one Gap junction channel and/or hemichannel blocker or inhibitor compound, alone or together with another cardiovascular therapeutic agent. In a preferred embodiment, the composition further comprises a pharmaceutically acceptable carrier or vehicle.

### Kits, Medicaments and Articles of Manufacture

A Gap junction channel and/or hemichannel blocker or inhibitor compound may also be used in the manufacture of the medicament for preventing and/or treating a cardiovascular disorder and related disorders and conditions.

In one aspect, the invention provides a kit for preventing and/or treating a cardiovascular disorder comprising one or more compositions or formulations described. For example, the invention includes a kit comprising a composition comprising a therapeutically effective amount of a Gap junction channel and/or hemichannel blocker or inhibitor compound, alone or in combination with one or more cardiovascular therapeutic agents. For example, the kit may include a composition comprising an effective amount of a Gap junction channel and/or hemichannel blocker or inhibitor compound and or more of the following: nitrates, β-blockers, calcium channel blockers (particularly for stable or unstable angina, but also for heart failure in the case of β-blockers); diuretic agents, vasodilator agents, positive inotropes, ACE inhibitors and aldosterone antagonists, e.g. spironolactone (particularly for heart failure); blood thinning therapeutics (e.g., aspirin, heparins, warfarins) and nitroglycerin (particularly for MI). Kits may also include compositions comprising or consisting essentially of a Gap junction channel and/or hemichannel blocker or inhibitor compound in alone or in combination with (e.g., in physical combination, provided as a combined preparation) one or more anti-thrombolytic therapies (e.g., streptokinase inhibitors, anti-platelet thereapetuics, such as, for example, clopidogrel). Kits may also include a gap junction channel and/or hemichannel blocker or inhibitor compound alone or in combination with (e.g., in physical combination, provided as a combined preparation).

Articles of manufacture are also provided comprising a vessel containing a composition or formulation of the invention (in any dose or dose form or device) as described herein and instructions for use for the treatment of a subject. For example, in another aspect, the invention includes an article of manufacture comprising a vessel containing a therapeutically effective amount of a Gap junction channel and/or hemichannel blocker or inhibitor compound, alone or in combination with one or more other cardiovascular therapeutic agents.

### Manufacture and Stability

The polypeptides of this invention can be manufactured using chemistries for known in the art. In one aspect, the formulations of this invention will comprise a salt of the polypeptides of this invention, such as the sodium salt of the polypeptides of this invention. In one embodiment the formulation may comprise the sodium salt of a polypeptides having any one of SEQ.ID.NO:1-52, for example.

In some embodiments, the formulations of this invention are substantially pure. By substantially pure is meant that the formulations comprise less than about 10%, 5%, or 1%, and preferably less than about 0.1%, of any amino acid or non-amino acid impurity. In some embodiments the total impurities, including metabolities of the connexin 43 modulating polypeptide, will be not more than 15%. In some embodiments the total impurities, including metabolities of the connexin 43 modulating modulating polypeptide, will be not more than 12%. In some embodiments the total impurities, including metabolities of the connexin 43 modulating modulating polypeptide, will be not more than 11%. In other embodiments the total impurities, including metabolities of the connexin 43 modulating polypeptide, will be not more than 10%.

In some embodiments, the purity of the formulations of this invention may be measured using a method selected from anion exchange HPLC (AEX-HPLC) or mass spectrometry. Mass spectrometry may include LC/MS, or LC/MS/MS. The assay may in some embodiments comprise both AEX-HPLC and LC/MS.

Sterile compositions comprising the connexin modulating polypeptide of this invention prepared using aseptic processing by dissolving the anti-connexin modulating polypeptide in the formulation vehicle. In one embodiment, the formulation may also be sterilized by filtration. Excipients used in the manufacture of of the formulations of this invention are widely used in pharmaceutical products and released to pharmacopeial standards. In one embodiment, the pharmaceutical compositions of this disclosure comprise a compound described herein and a pharmaceutically acceptable carrier that comprises a sterile excipient. In one embodiment, the pharmaceutical composition comprises a compound selected from SEQ ID NOS: 1-10, 21, 25, 27-30, 46, 49, 50, and 51; and the pharmaceutically acceptable carrier comprises a sterile excipient. In one embodiment, the sterile excipient is a sterilized form of the excipients described herein.

In one aspect the invention is directed to sustained administration of a Gap junction channel and/or hemichannel blocker or inhibitor compound and, optionally, antoher cardiovascular therapeutic agent. In one embodiment, the agent(s) are administered for at least about 0.5 hours, about 1-24 hours, at least about 2, hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours or at least about 24 hours.

Any of the methods of treating a subject having or suspected of having or predisposed to a disease, disorder, and/or condition referenced or described herein may utilize the administration of any of the doses, dosage forms, formulations, compositions and/or devices herein described.

A better understanding of the invention will be gained by reference to the following non-limiting experimental section which is illustrative and is not intended to limit the invention or the claims in any way. The data support the use of the compounds and compositions described herein for treatment of cardiovascular diseases, disorders and conditions, as described.

### EXAMPLES

### Methods

Cell Culture

Human cerebral microvascular endothelial (hCMVEC) cells (ABMGood, (O'Carroll et al., 2015, Journal of neuroinflammation 12:131) were grown in M199 media (Gibco), supplemented with 10% FCS 1 μg/mL hydrocortisone (Sigma), 3 μg/mL human FGF (Peprotech), 10 μg/mL human EGF (Peprotech), 10 μg/mL heparin (Sigma), 2 mM Glutamax (Gibco) and 80 μM butyryl cAMP (Sigma), and cultured in 1 μg/cm² collagen I (Gibco) coated T25 or T75 flasks. ARPE-19 cells were cultured in DMEM/F:12, 10% FCS and 1% Antibiotic-antimycotic (Thermo fisher scientific). All cells were grown to 80-90% confluence; trypsinised using TrypLE Express (Life technologies); split at 1:3-1:5 ratio, and cultured at 37° C., 95% O₂ and 5% CO₂ unless stated otherwise.

Peptides and Chemical Reagents

For cell scrape load—dye coupling assays of gap junctions, induced protein distribution changes and ATP release studies of hemichannel block, Connexin43 mimetic peptide (Peptide5, sequence VDCFLSRPTEKT (SEQ ID NO: 45)) (Auspep, Australia) was synthesized at purity >95% and dissolved in miliQ H₂O at a stock concentration of 10 mM. For competition assays extracellular loop matching peptides were custom synthesised and dissolved in miliQ H₂O at a stock concentration of 1 mM according to purity supplied by the manufacturer (Auspep, Australia). For Peptide5 sequence substitution studies, peptides were generated in the School of Chemical Sciences, University of Auckland. The peptides were prepared using Fmoc solid phase synthesis. Fmoc-Thr(tBu)-OH or Fmoc-Ala-OH pre-loaded to the HMPP linker and attached to in-house prepared aminomethyl PS resin 2.1 (using DIC in DMF). This was followed by alternating de-protection and coupling reactions using 5% piperazine containing 0.1 M 6-Cl-HOBt and HBTU and NMM respectively via automated Tribute Fmoc-SPPS. Resin cleavage using TFA afforded crude products, which underwent purification via semi-preparative RP-HPLC. Peptides were obtained in high purity (>95%) and with good yields (17%-46%). All alanine substituted analogues were miliQ H₂O soluble (Table 5).

TABLE 5
Single alanine substituted peptides of Connexin43
to investigate Peptide5 sequence specificty.
Alanine substitutions are marked in underline.

| Single alanine | | |
|---|---|---|
| substituted peptides | Sequence | SEQ ID NO: |
| Ala-1 | ADCFLSRPTEKT | 1 |
| Ala-2 | VACFLSRPTEKT | 78 |
| Ala-3 | VDAFLSRPTEKT | 79 |
| Ala-4 | VDCALSRPTEKT | 2 |
| Ala-5 | VDCFASRPTEKT | 80 |
| Ala-6 | VDCFLARPTEKT | 3 |
| Ala-7 | VDCFLSAPTEKT | 81 |
| Ala-8 | VDCFLSRATEKT | 82 |
| Ala-9 | VDCFLSRPAEKT | 4 |
| Ala-10 | VDCFLSRPTAKT | 83 |
| Ala-11 | VDCFLSRPTEAT | 84 |
| Ala-12 | VDCFLSRPTEKA | 5 |

### Example 1

### Synthesis of Truncated Peptide5 Motifs

In order to determine motifs of Peptide5 that are essential for function, A further six truncated peptides (Table 2) were synthesized at a purity of 95% and dissolved in miilQ H₂O at a stock concentration of 10 mM (ChinaPeptide Co Ltd, Shanghai, China; www.chinapeptide.org).

TABLE 2
Truncated Peptides of Connexin43 peptides

| Truncated peptides | Sequence | SEQ ID NO: |
|---|---|---|
| Modification 1 | CFLSRPTEKT | 85 |
| Modification 2 | LSRPTEKT | 86 |
| Modification 3 | SRPTEKT | 77 |
| Modification 4 | VDCFLSRPTE | 87 |
| Modification 5 | VDCFLSRP | 88 |
| Modification 6 | VDCFLS | 89 |

A scrambled sequence of Peptide5 (RFKPSLCTTDEV (SEQ ID NO: 90)) was used as peptide control (Auspep, Australia) (although this sequence has homology with a cytoskeleton protein and could have non-specific effects). A non-specific hemichannel inhibitor, Lanthanum chloride (LaCl₃, Sigma) (Mylvaganam et al., 2014, Frontiers in physiology 5:172) was dissolved in miliQ H₂O at a stock concentration of 1 mM. Carbenoxolone (CBX, Sigma), a non-specific inhibitor of connexin channels, and also a broad-spectrum inhibitor of gap junction channels (Salameh and Dhein, 2005, Biochim Biophys Acta 1719:36-58) was dissolved in miliQ H₂O at a stock concentration of 10 mM.

### Example 2

### Determination of Motifs of Peptide5 for Function

In order to determine motifs of Peptide5 that are essential for function, the six truncated peptides (Table 2) were synthesized at a purity of 95% and dissolved in miilQ H₂O at a stock concentration of 10 mM (ChinaPeptide Co Ltd, Shanghai, China; www.chinapeptide.org).

A scrambled sequence of Peptide5 (RFKPSLCTTDEV (SEQ ID NO: 90)) was used as peptide control (Auspep, Australia) (although this sequence has homology with a cytoskeleton protein and could have non-specific effects). A non-specific hemichannel inhibitor, Lanthanum chloride (LaCl₃, Sigma) (Mylvaganam et al., 2014) was dissolved in miliQ H₂O at a stock concentration of 1 mM. Carbenoxolone (CBX, Sigma), a non-specific inhibitor of connexin channels, and also a broad-spectrum inhibitor of gap junction channels (Salameh and Dhein, 2005) was dissolved in miliQ H₂O at a stock concentration of 10 mM.

Scrape Loading Assay

The hCMVEC cells were plated in 1 μg/cm² collagen I (Gibco) 12-well plate at a density of 0.4×10⁶ cells per well, and cultured overnight. A confluent monolayer of hCMVECs was pre-incubated in Peptide5 (5-100 μM) dissolved in culture medium for 2 hours or 24 hours. CBX at a concentration of 100 μM was used as a positive control. Cells were washed three times with phosphate buffered saline (PBS) without Ca²⁺ or Mg²⁺. The cells were then incubated in 0.05% Lucifer Yellow (LY) (Sigma), a fluorescent dye that is transferred through coupled gap junction channels (el-Fouly et al., 1987, Experimental cell research 168:422-430), +/−Peptide5 dissolved in PBS, and scrape-wounded with a size 10 carbon steel surgical blade (Swann-Morton, England). Following 5-minute incubation at 37° C. in 95% O₂ and 5% CO₂ without light, the 0.05% LY solution was removed. Cells were rinsed four times with PBS with Ca²⁺ or Mg²⁺, and then fixed in 4% paraformaldehyde (PFA) in PBS at pH 7.4 for 10 minutes at room temperature. Cells were then washed 3 times in PBS to remove PFA before fluorescent imaging. Fluorescent images were visualised using a Nikon TE2000E inverted fluorescent microscope (10× magnification, 0.3 numerical aperture), and captured using a Digital Sight CCD camera and Eclipse Net software (Nikon). Three images within each well from three independent experiments were taken for analysis and the total number of cells showing dye uptake from those that had been loaded were counted manually by masked observers.

Immunocytochemistry and Quantification of Cx43 Plaques

ARPE-19 cells were grown until confluent in 8-well glass chamber-slides (BD Falcon). Confluent monolayers of ARPE-19 cells were incubated at a final concentration of 50 to 500 μM Peptide5, 500 μM LaCl₃ and 500 μM CBX in culture medium for 2 or 6 hours. Cells were fixed in 4% PFA (ProSciTech) at pH 7.4 for 10 min at room temperature, permeabilized with 0.05% Triton-X100 in PBS, and incubated in 10% normal goat serum to block non-specific labelling. Cells were rinsed three times with PBS containing 0.1 mM CaCl₂ between each fixation, permeabilization, and blocking steps. Connexin43 polyclonal rabbit antibody (C6219, Sigma, 1:2000) was applied overnight, followed by a goat anti-rabbit Alexa Fluor® 568 secondary antibody (Invitrogen, 1:200) for 45 minutes. Nuclei were counterstained with DAPI (Invitrogen) at 10,000-fold dilution for 5 mins, and mounted with Citifluor™ mounting medium. Images were visualised and captured using an oil immersion lens (60× magnification, 1.35 numerical aperture) on an Olympus FV1000 upright confocal laser scanning microscope and Olympus FV10-ASW 4.0 software. The Transfluor feature in MetaXpress® Image acquisition and analysis software (Version 5.3.0.1, Molecular Devices) was used to quantify the total area of Connexin43 plaques per image. Results represent mean ±standard error and statistical tests were conducted using one-way ANOVA and Tukey's multiple comparisons test.

In Vitro Ischemia-Reperfusion ATP Assay

The human cerebral endothelial cells (hCMVEC) were plated at a density of 0.025×10⁶ cells per well in a collagen-coated 12-well plate (1 μg/cm²) a day prior to the experiment. Cells were incubated in culture medium overnight. Hypoxic, acidic, ion-shifted Ringer injury solution that mimics ionic concentrations and acid-base shifts of the interstitial space in hypoxic-ischemic brains (Bondarenko and Chesler, 2001, Current medicinal chemistry 17:4191-4230) was used to trigger hemichannel opening. The injury solution contained (in mM): 38 NaCl, 13 NaHCO₃, 3 Na-gluconate, 65 K-gluconate, 38 NMDG-Cl, 1 NaH₂PO₄, and 1.5 MgCl₂. The injury solution was bubbled in 95% N₂, 5% CO₂ gas (20 L/min) for 5 minutes and pH adjusted to 6.6 before use. The standard ringer solution contained (in mM): 124 NaCl, 3 KCl, 26 NaHCO₃, 26 NaHCO₃, 1 NaH₂PO₄, 1.3 CaCl₂, 1.5 MgCl₂, and 10 Glucose, and pH adjusted to 7.4 before use (Bondarenko and Chesler, 2001). In the ischemia injury model, hCMVEC cells were incubated for 2 hours in 500 μL injury solution+/−Peptide5 (100 μM) (O'Carroll et al., 2008, Cell communication & adhesion 15:27-42; Danesh-Meyer et al., 2012, Brain: a journal of neurology 135:506-520; Davidson et al., 2012, Annals of neurology 71:121-132; Davidson et al., 2013, Experimental neurology 248:301-308; Davidson et al., 2014, PloS one 9:e96558; Davidson et al., 2015, J Cereb Blood Flow Metab), or +/−extracellular loop sequences (Table 5), or modified peptides (Table 2, 3) dissolved in injury solution (500 μL) with a final concentration of 100 μM. For negative control, hCMVEC cells were incubated only in 500 μL standard ringer solution for 2 hours. For positive control, hCMVEC cells were incubated only in 500 μL standard ringer solution for 2 hours. All incubations were conducted at 37° C. in 95% O₂ and 5% CO₂. At the end of each experiment, samples were removed and immediately placed on ice. The concentration of ATP in the samples was determined using a Luciferin/luciferase bioluminescence reaction (ATP Determination Kit, Molecular Probes) and detected using a luminescence plate reader (VICTOR X, Perkin Elmer #2030-0010). Standard curves were generated in each experiment from an ATP standard (0 to 500 nM) to convert bioluminescence units to ATP concentration. Treatment groups had a sample size of 2 wells per experiment, and the concentration of ATP in each sample was measured in triplicates over ten repeated readings. The data is presented as mean±standard error relative to the injury or injury-reperfusion positive control. Statistically significant differences between samples were tested using one-way analysis of variance and Tukey's multiple comparisons test.

Single Alanine Substituted Peptides and Truncated Peptides Assay

Peptides listed in table 5 and 2 were assessed using the ischemia injury model in hCMVEC cells as described above, and the medium was collected for ATP release measurement. For these experiments a standard 100 μM concentration was used, which is slightly higher than the 5-50 μM known to induce hemichannel block (O'Carroll et al., 2008). This was to ensure that any results with peptides that might have reduced functional efficacy would still fall within a measurable range.

Competitive Peptide Binding Assay

Peptide5 interacting site on the Connexin43 protein was determined using a competition assay. Extracellular loop sequences (Auspep, Australia) were dissolved in miliQ H₂O were at a stock concentration of 1 mM (Table 3). Extracellular loop sequences were mixed with the native Peptide5 at 1:1 ratio (100 μM), which was left for 1 hour at room temperature prior to the experiment to allow competitive binding to occur. Following 1-hour incubation, extracellular loop sequence and peptide5 mixture was dissolved in injury solution and exposed to hCMVEC cells for 2 hours using the ischaemia-reperfusion model described above. Extracellular medium was then collected for ATP measurement. All extracellular loop peptides were soluble in miliQ H₂O except for the EL2a sequence, which was not used for further studies.

TABLE 3
Extracellular loop sequences of Connexin43 used
in competition assays

| Extracellular | | SEQ |
|---|---|---|
| loop peptides | Sequence | ID NO: |
| EL1 complete | ESAWG DEQSA FRCNT QQPGC | 91 |
| | ENVCY DKSFP ISHVR | |
| EL2 truncated | Q WYIYG FSLSA VYTCK RDPCP | 92 |
| | HQ [symbol] | |
| EL1a | ESAWG DEQSA FRCNT QQ | 93 |
| EL1b | PGC ENVCY DKSFP ISHVR | 94 |
| EL1c | A FRCNT QQPGC ENVCY D | 95 |
| *EL2a | FLLIQ WYIYG FSLSA VYTC | 96 |
| EL2b | K RDPCP HQ [symbol] | 97 |
| EL2c | G FSLSA VYTCK RDPCP HQ [symbol] | 98 |

*EL2a sequence was not used in this study.

Scrape Loading Assay

The hCMVEC cells were plated in 1 μg/cm² collagen I (Gibco) 12-well plate at a density of 0.4×10⁶ cells per well, and cultured overnight. A confluent monolayer of hCMVECs was pre-incubated in Peptide5 (5-100 μM) dissolved in culture medium for 2 hours or 24 hours. CBX at a concentration of 100 μM was used as a positive control. Cells were washed three times with phosphate buffered saline (PBS) without Ca²⁺ or Mg²⁺. The cells were then incubated in 0.05% Lucifer Yellow (LY) (Sigma), a fluorescent dye that is transferred through coupled gap junction channels (el-Fouly et al., 1987), +/−Peptide5 dissolved in PBS, and scrape-wounded with a size 10 carbon steel surgical blade (Swann-Morton, England). Following 5-minute incubation at 37° C. in 95% O₂ and 5% CO₂ without light, the 0.05% LY solution was removed. Cells were rinsed four times with PBS with Ca²⁺ or Mg²⁺, and then fixed in 4% paraformaldehyde (PFA) in PBS at pH 7.4 for 10 minutes at room temperature. Cells were then washed 3 times in PBS to remove PFA before fluorescent imaging. Fluorescent images were visualised using a Nikon TE2000E inverted fluorescent microscope (10× magnification, 0.3 numerical aperture), and captured using a Digital Sight CCD camera and Eclipse Net software (Nikon). Three images within each well from three independent experiments were taken for analysis and the total number of cells showing dye uptake from those that had been loaded were counted manually by masked observers.

### Immunocytochemistry and Quantification of Cx43 Plaques

ARPE-19 cells were grown until confluent in 8-well glass chamber-slides (BD Falcon). Confluent monolayers of ARPE-19 cells were incubated at a final concentration of 50 to 500 μM Peptide5, 500 μM LaCl₃ and 500 μM CBX in culture medium for 2 or 6 hours. Cells were fixed in 4% PFA (ProSciTech) at pH 7.4 for 10 min at room temperature, permeabilized with 0.05% Triton-X100 in PBS, and incubated in 10% normal goat serum to block non-specific labelling. Cells were rinsed three times with PBS containing 0.1 mM CaCl₂ between each fixation, permeabilization, and blocking steps. Connexin43 polyclonal rabbit antibody (C6219, Sigma, 1:2000) was applied overnight, followed by a goat anti-rabbit Alexa Fluor® 568 secondary antibody (Invitrogen, 1:200) for 45 minutes. Nuclei were counterstained with DAPI (Invitrogen) at 10,000-fold dilution for 5 mins, and mounted with Citifluor™ mounting medium. Images were visualised and captured using an oil immersion lens (60× magnification, 1.35 numerical aperture) on an Olympus FV1000 upright confocal laser scanning microscope and Olympus FV10-ASW 4.0 software. The Transfluor feature in MetaXpress® Image acquisition and analysis software (Version 5.3.0.1, Molecular Devices) was used to quantify the total area of Connexin43 plaques per image. Results represent mean±standard error and statistical tests were conducted using one-way ANOVA and Tukey's multiple comparisons test.

In Vitro Ischemia-Reperfusion ATP Assay

The human cerebral endothelial cells (hCMVEC) were plated at a density of 0.025×10⁶ cells per well in a collagen-coated 12-well plate (1 μg/cm²) a day prior to the experiment. Cells were incubated in culture medium overnight. Hypoxic, acidic, ion-shifted Ringer injury solution that mimics ionic concentrations and acid-base shifts of the interstitial space in hypoxic-ischemic brains (Bondarenko and Chesler, 2001) was used to trigger hemichannel opening. The injury solution contained (in mM): 38 NaCl, 13 NaHCO₃, 3 Na-gluconate, 65 K-gluconate, 38 NMDG-Cl, 1 NaH₂PO₄, and 1.5 MgCl₂. The injury solution was bubbled in 95% N₂, 5% CO₂ gas (20 L/min) for 5 minutes and pH adjusted to 6.6 before use. The standard ringer solution contained (in mM): 124 NaCl, 3 KCl, 26 NaHCO₃, 26 NaHCO₃, 1 NaH₂PO₄, 1.3 CaCl₂, 1.5 MgCl₂, and 10 Glucose, and pH adjusted to 7.4 before use (Bondarenko and Chesler, 2001). In the ischemia injury model, hCMVEC cells were incubated for 2 hours in 500 μL injury solution+/31 Peptide5 (100 μM) (O'Carroll et al., 2008, Danesh-Meyer et al., 2012, Davidson et al., 2012, Davidson et al., 2013b, Davidson et al., 2014, Davidson et al., 2015), or +/−extracellular loop sequences (Table 5), or modified peptides (Table 2, 3) dissolved in injury solution (500 μL) with a final concentration of 100 For negative control, hCMVEC cells were incubated only in 500 μL standard ringer solution for 2 hours. For positive control, hCMVEC cells were incubated only in 500 μL standard ringer solution for 2 hours. All incubations were conducted at 37° C. in 95% O₂ and 5% CO₂. At the end of each experiment, samples were removed and immediately placed on ice. The concentration of ATP in the samples was determined using a Luciferin/luciferase bioluminescence reaction (ATP Determination Kit, Molecular Probes) and detected using a luminescence plate reader (VICTOR X, Perkin Elmer #2030-0010). Standard curves were generated in each experiment from an ATP standard (0 to 500 nM) to convert bioluminescence units to ATP concentration. Treatment groups had a sample size of 2 wells per experiment, and the concentration of ATP in each sample was measured in triplicates over ten repeated readings. The data is presented as mean±standard error relative to the injury or injury-reperfusion positive control. Statistically significant differences between samples were tested using one-way analysis of variance and Tukey's multiple comparisons test.

Single Alanine Substituted Peptides and Truncated Peptides Assay

Peptides listed in Table 5 and 2 were assessed using the ischemia injury model in hCMVEC cells as described above, and the medium was collected for ATP release measurement. For these experiments a standard 100 μM concentration was used, which is slightly higher than the 5-50 μM known to induce hemichannel block (O'Carroll et al., 2008). This was to ensure that any results with peptides that might have reduced functional efficacy would still fall within a measurable range.

Competitive Peptide Binding Assay

Peptide5 interaction sites on the Connexin43 protein was determined using a competition assay. Extracellular loop sequences (Auspep, Australia) were dissolved in miliQ H₂O were at a stock concentration of 1 mM (Table 3). Extracellular loop sequences were mixed with the native Peptide5 at 1:1 ratio (100 μM), which was left for 1 hour at room temperature prior to the experiment to allow competitive binding to occur. Following 1-hour incubation, extracellular loop sequence and peptide5 mixture was dissolved in injury solution and exposed to hCMVEC cells for 2 hours using the ischaemia-reperfusion model described above. Extracellular medium was then collected for ATP measurement. All extracellular loop peptides were soluble in miliQ H₂O except for the EL2a sequence.

### Results

High Concentration of Peptide5 Causes Internalization of Connexin43 Plaques from ARPE-19 Membrane

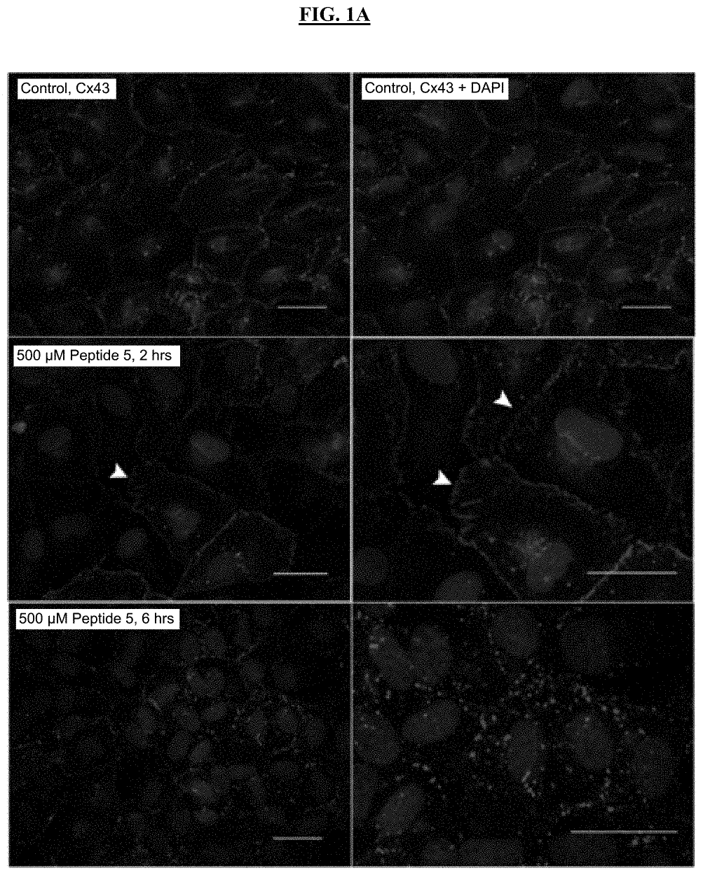

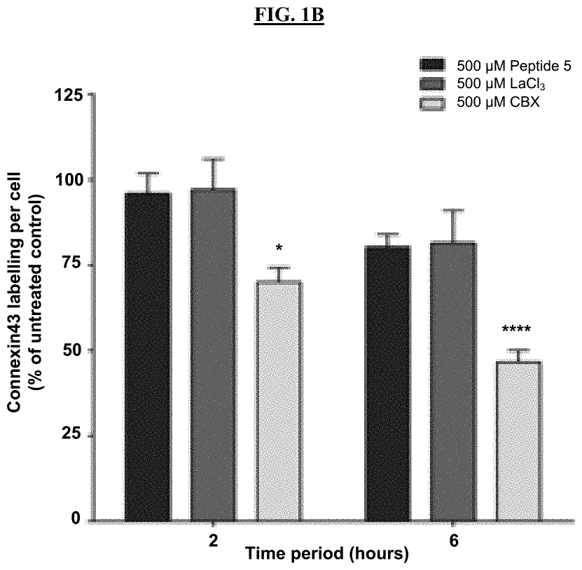

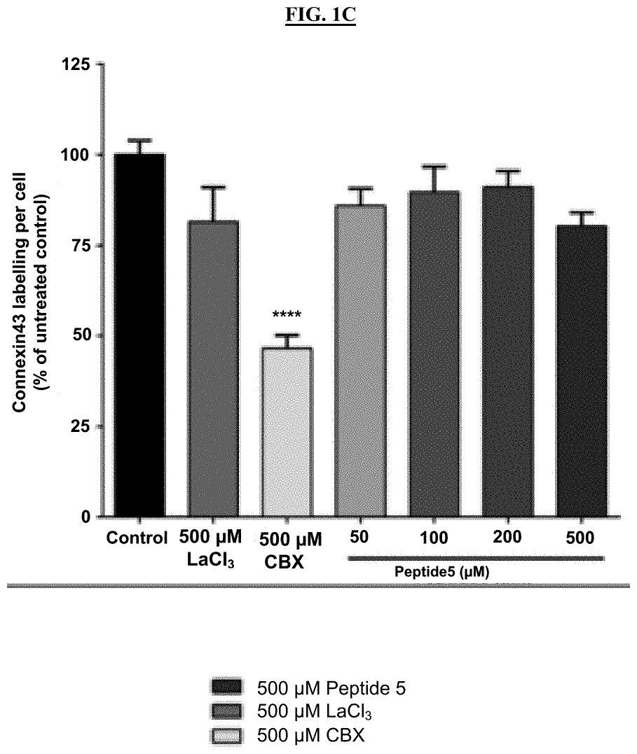

Peptide5 at low concentrations (5-50 μM) specifically inhibits hemichannels, but high concentration (500 μM) inhibits both hemichannel and gap junction channel function (O'Carroll et al., 2008). The inventors exposed ARPE-19 cells, a cell line with high endogenous expression of connexin43 at cell-to-cell borders (Hutnik et al., 2008, Investigative ophthalmology & visual science 49:800-806), to Peptide5 (500 μM) and determined its effect on the distribution and number of connexin43 gap junction plaques (FIGS. 1A, 1B and 1C). FIG. 1A shows immunofluorescent connexin43 plaques labelled in a regular tile-like pattern between cell-to-cell contacts in basal conditions. In addition, connexin43 labelling was also visible in the perinuclear region, which most likely represents an intracellular pool of Connexin43 in the Golgi apparatus (Das Sarma et al., 2001, Journal of cell science 114:4013-4024. Following 2-hour exposure to Peptide5 (500 μM), Connexin43 plaques were visible along cell-to-cell contacts but showed patterns of cytoplasmic streaming that extended from the membrane towards the center of the cell (FIG. 1A). Punctate connexin43 labeling was also visible in the intracellular region immediately below the plasma membrane (FIG. 1A). Connexin43 plaques along the cell-to-cell interface were fragmented following 6-hour exposure to Peptide5 (500 μM) (FIG. 1A).

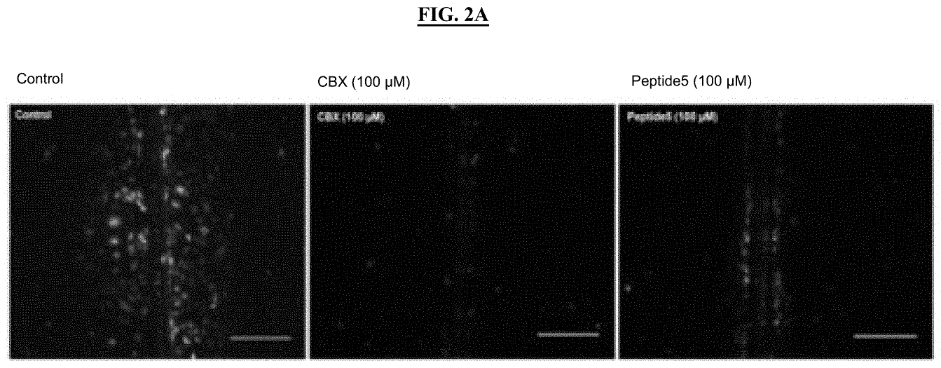

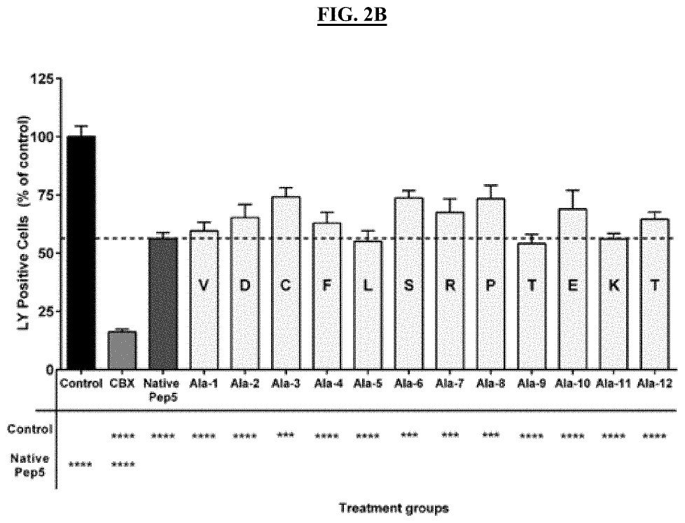

The inventors also surprisingly determined whether the total Connexin43 labeling per cell was reduced following exposure to Peptide5. No significant differences in Connexin43 labelling per cell were observed following 2-or 6-hour treatment with Peptide5 (50-500 μM) (p>0.1). Likewise, LaCl3 (500 μM) was not significantly different to the control post 2- or 6-hour (p>0.1) treatment (FIGS. 1B and 1C). In contrast, CBX (500 μM) significantly reduced the total Connexin43 expression by 30.0±8.2% (p=0.0043) following 2-hour exposure and by 53.4±8.1% (p<0.0001) after 6 hours relative to the control (FIGS. 2A and 2B).

Alanine Substitutions does not Alter Peptide5 Mediated Gap Junction Uncoupling

FIGS. 2A and 2B summarize the effects of native and modified Peptide5 on gap junction communication in vitro. In control, LY positive hCMVEC cells indicated cell-to-cell LY transfer through gap junction channels, which was reduced following treatment with CBX and Peptide5 (FIG. 2A). CBX and native Peptide5 significantly reduced LY positive cells to 16.3±1.1% (p<0.0001) and 56.2±2.6% (p<0.0001) compared to the control (FIG. 2A). When compared to native Peptide5, there was no significant difference in LY positive cells with alanine modifications (p>0.9) (FIG. 2B).

### Peptide5 Mediated Hemichannel Block is a Via a Precise Sequence Specific Mechanism

To determine the Peptide5-mediated hemichannel block mechanism, the inventors systematically substituted an alanine into Peptide5 (Table 5) to determine which amino acids are important for efficacy. CBX and LaCl3 controls significantly attenuated ATP to 9.1±10.1% (p<0.0001) and 47.8±13.6% (p<0.0001) respectively of the injury control (100±3.6%) (FIG. 3). Following treatment with native Peptide5, ATP release was significantly reduced to 44.6±7.7% of the injury control (p<0.0001) (FIG. 3). Loss of function was observed with alanine substitutions Ala-2, Ala-3, Ala-4, Ala-5, Ala-6, Ala-7, Ala-8 and Ala-11 as these were not significantly different to the injury control (p>0.9) (FIG. 3). In contrast, Ala-4, Ala-9, Ala-10, and Ala-12 substitutions significantly lowered ATP release to 60.7±4.3% (p=0.0098), 62.4±12.4%, (p=0.0154), 62.0±9.0% (p=0.0137), 61.2±9.8% (p=0.02) of the injury control respectively (FIG. 3), which indicates that these peptides retained efficacy. Therefore, Ala-4, Ala-9, Ala-10 and Ala-12 substituted peptides were not significantly different to the native Peptide5 (p>0.09). Ala-1 was not significantly different when compared to injury control or the native Peptide5.

### SRPTEKT Motif (SEQ ID NO: 77) is Crucial for Peptide5 Mediated Inhibition of Hemichannels but on its Own is Not Effective

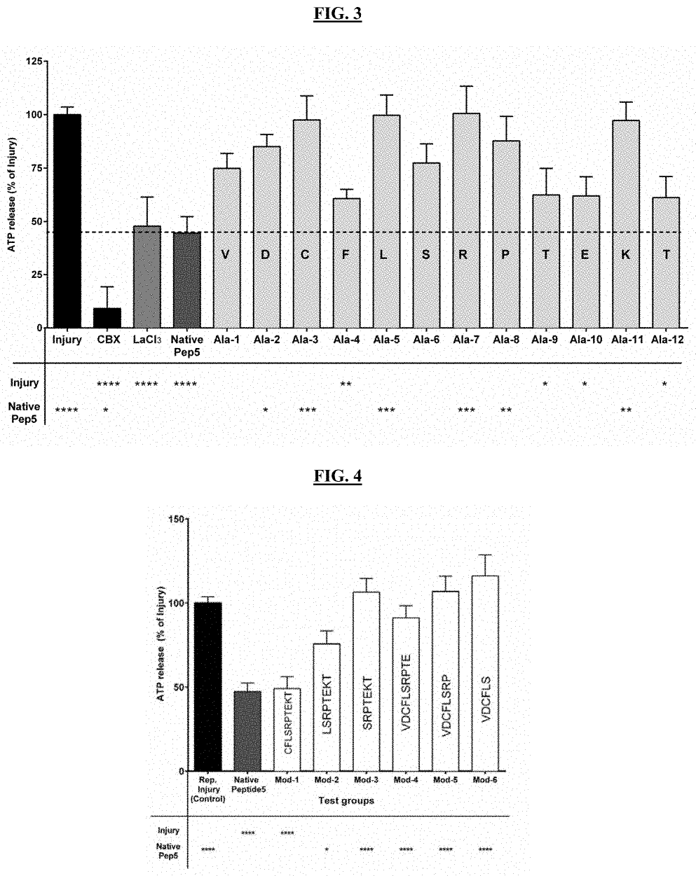

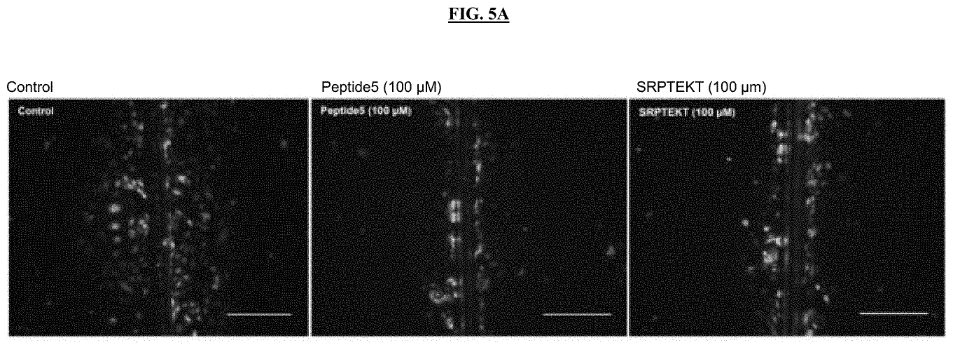

To determine the functional region(s) of Peptide5, truncated Peptide5 sequences were tested in the ATP hemichannel assay (Table 2, FIG. 4). The native Peptide5 sequence significantly reduced ATP to 47.3±5.1% of the injury control (100±3.7%) (p<0.0001). Peptides mod-2, mod-3, mod-4, mod-5 and mod-6 were not significantly different to the injury control indicating loss of function (FIG. 4). In another words, mod-2, mod-3, mod-4, mod-5 and mod-6 were significantly less effective in its ability to inhibit ATP release by 28.4±9.8% (p=0.0231), 59.2±8.8% (p<0.0001), 43.9±8.8% (p<0.0001), 59.6±11.5% (p<0.0001) and 68.8±9.8% (p<0.0001) compared to the native Peptide5 respectively. Interestingly, mod-1 (49.1±7.1%) was significantly different to the injury control (p<0.0001) but not significantly different to native peptide5 (p>0.9) showing it retained function.

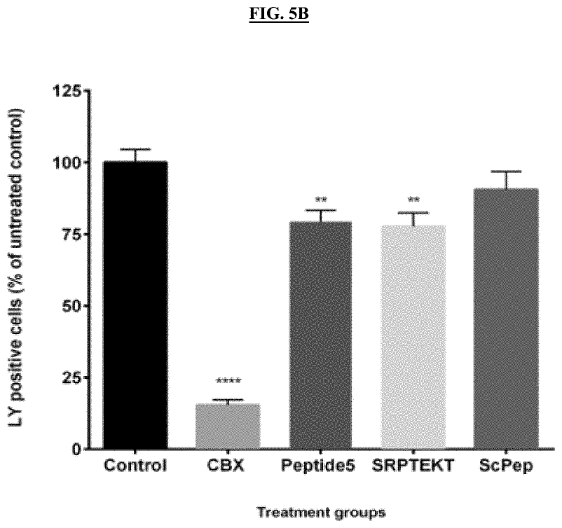

These results indicate that Peptide5 mediated inhibition of ATP release is sequence specific, and the conserved SRPTEKT region (SEQ ID NO: 77) appears to be important for Peptide5 function but on its own is not effective for hemichannel inhibition. The inventors also surprisingly determined whether SRPTEKT sequence (SEQ ID NO: 77) on its own, at higher concentration, could inhibit gap junction communication in the scrape-loading assay (FIGS. 5A and 5B). CBX (carbenoxelone significantly reduced LY dye transfer to 15.3±1.8% (p<0.0001) of the untreated control (100±4.5%) (FIGS. 5A and 5B). Compared to untreated control, LY dye transfer was significantly reduced to 79.4±3.9% (p=0.0057) and 77.6±4.8% (p=0.0028) with Peptide5 and SRPTEKT (SEQ ID NO: 77) treatment respectively (FIGS. 5A and 5B). There was no significant difference with scrambled peptide relative to untreated control (p=0.37).

### Site of Action Competition Assay

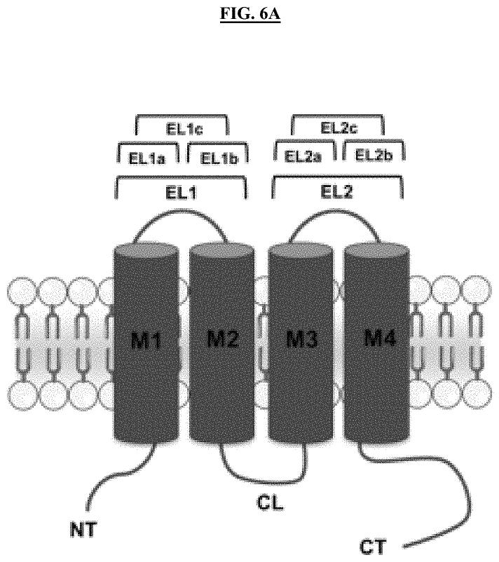

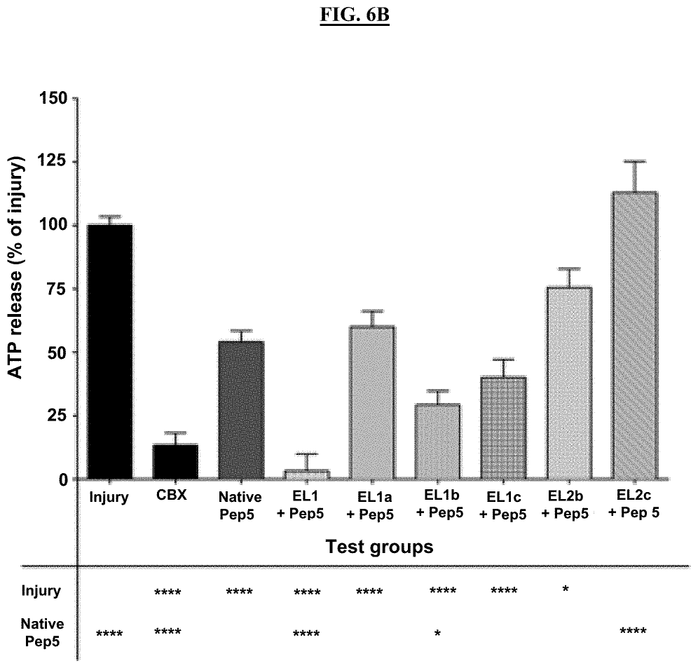

To determine the site of action, synthetic peptide fragments (Table. 3) derived from the extracellular loop 1 and 2 of the connexin43 protein was tested against the native Peptide5. Because a fraction of a peptide segment competitively binds with the native Peptide5 sequence, there is observed a loss of Peptide5 function, and hemichannel mediated ATP release is greater when compared to Peptide5 alone. FIGS. 6A and 6B demonstrate that CBX and Peptide5 significantly reduced ATP release to 13.4±4.8% (p<0.0001) and 54.1±4.4% (p<0.0001) of the injury control (100±3.5%) respectively. Segments EL1 complete, EL1a, EL1b and EL1c and EL2b sequences in combination with Peptide5 significantly lowered ATP to 3.2±6.7% (p<0.0001), 60.1±6.2% (p<0.0001), 29.3±5.5% (p<0.0001), 40.1±7.0% (p<0.0001) and 75.5±7.4% (p=0.0302) of the injury control respectively (FIGS. 6A and 6B). Surprisingly, the inventors have discovered that EL1 complete and ELlb sequences in combination with Peptide5 were significantly more effective by 50.8±7.9% (p<0.0001) and 24.8±8.8% (p=0.0334) compared to Peptide5 alone (FIGS. 6A and 6B). In contrast, EL2c completely abolished Peptide5 function as it was not significantly different to the injury control (p=0.5615), and the ATP released was significantly greater by 58.9±8.8% (p<0.0001) than Peptide5 alone. These results indicate that EL2c sequence is likely to be the site of action for Peptide5.

### Example 3

### Formulation of Gap Junction and/or Connexin Channel Modulating Peptides

This invention provides a formulation containing one or more gap junction and/or connexin channel modulating peptides. The invention provides an aqueous formulation of the gap junction and/or connexin channel modulating peptides that is suitable for therapeutic use and remains stable under normal use storage conditions for an extended period of time. The formulation is useful for treating conditions in which treatment with a gap junction and/or connexin modulating peptide would provide a therapeutic benefit. For topical administration, one to two drops of these formulations can be delivered to the affected area one to six times per day.

For the administration of the connexin modulating peptides, the connexin 43 modulating peptides (modulator) may be present in the formulation at about 8 μM to about 20 μM final concentration, and alternatively the connexin 43 modulator is present at about 10 μM to about 20 μM final concentration, or at about 10 to about 15 μM final concentration. In certain other embodiments, the connexin 43 modulator is present at about 10 μM final concentration. In yet another embodiment, the connexin 43 modulator is present at about 1-15 μM final concentration. In other embodiments, the connexin 43 modulator is present at about a 20 μM, 30 μM, 40 μM, 50 μM, 60 μM, 70 μM, 80 μM, 90 μM, 100 μM, 10-200 μM, 200-300 μM, 300-400 μM, 400-500 μM, 500-600 μM, 600-700 μM, 700-800 μM, 800-900 μM, 900-1000 or 1000-1500 μM, or 1500 μM-2000 μM, 2000 μM-3000 μM, 3000 μM-4000 μM, 4000 μM-5000 μM, 5000 μM-6000 μM, 6000 μM-7000 μM, 7000 μM-8000 μM, 8000 μM-9000 μM, 9000 μM-10,000 μM, 10,000 μM-11,000 μM, 11,000 μM-12,000 μM, 12,000 μM-13,000 μM, 13,000 μM-14,000 μM, 14,000 μM-15,000 μM, 15,000 μM-20,000 μM, 20,000 μM-30,000 μM, 30,000 μM-50,000 μM, or greater, or any range or subrange between any two of the recited doses, or any dose falling within the range of from about 20 μM to about 50,000 μM.

For the administration of the connexin 43 modulator, the connexin modulator is any of the peptide sequences described herein. In some embodiments, the connexin modulator can be the peptide SEQ ID NO: 1.

Still other dosage levels between about 1 nanogram (ng)/kg and about 1 mg/kg body weight per day of each of the modulating peptides. In certain embodiments, the dosage of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 0.01 mg per kg body weight, about 0.01 mg to about 0.1 mg per kg body weight, or about 0.1 mg to about 1 mg per kg body weight. In certain embodiments, the dosage of each of the subject compounds will generally be in the range of about 0.001 mg to about 0.01 mg per kg body weight, about 0.01 mg to about 0.1 mg per kg body weight, about 0.1 mg to about 1 mg per kg body weight. If more than one gap junction and/or connexin channel modulating peptides is used, the dosage of each anti-connexin agent need not be in the same range as the other. For example, the dosage of one gap junction and/or connexin channel modulator may be between about 0.01 mg to about 10 mg per kg body weight, and the dosage of another gap junction and/or connexin channel modulator may be between about 0.1 mg to about 1 mg per kg body weight, 0.1 to about 10, 0.1 to about 20, 0.1 to about 30, 0.1 to about 40, or between about 0.1 to about 50 mg per kg body weight. The dosage may also be about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, 50.0, 52.5, 55.0, 57.5, 60.0, 62.5, 65.0, 67.5, 70.0, 72.5, 75.0, 77.5, 80.0, 82.5, 85.0, 87.5, 90.0, 92.5, 95.0, 97.5, or about 100.0 mg per kg body weight, or any range or subrange between any two of the recited doses, or any dose falling within the range of from about 0.1 to about 100 mg per kg body weight.

Optionally, the formulation can include a viscosity enhancer to increase the residence time of the formulation in the affected adminstration site. As a non-limiting example, hydroxypropyl methyl cellulose, can be used as a viscosity enhancer for the present invention.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A pharmaceutical composition comprising a compound selected from the group consisting of SEQ ID NOS:1-52 and a pharmaceutically acceptable carrier.

2. A pharmaceutical composition comprising a compound according to Formula I, and a pharmaceutically acceptable carrier.

3. A pharmaceutical composition comprising a compound according to Formula II, and a pharmaceutically acceptable carrier.

4. A pharmaceutical composition comprising a compound according to Formula III, and a pharmaceutically acceptable carrier.

5. A pharmaceutical composition comprising a compound according to Formula IV, and a pharmaceutically acceptable carrier.

6. An isolated compound selected from the group consisting of SEQ ID NOS:1-52.

7. An isolated compound selected according to Formula I.

8. An isolated compound selected according to Formula II.

9. An isolated compound selected according to Formula III.

10. An isolated compound selected according to Formula IV.

11. A pharmaceutical composition of claim 1, wherein the compound is selected from SEQ ID NOS: 1-10, 21, 25, 27-30, 46, 49, 50, and 51; and the pharmaceutically acceptable carrier comprises a sterile excipient.

12. An isolated compound according to SEQ ID NO:43.

13. A pharmaceutical composition comprising a compound according to SEQ ID NO:13 and a pharmaceutically acceptable carrier.


SEQUENCE LISTING (98 sequences)

SEQ ID NO: 1 - protein, 12 aa, Artificial Sequence
  Description of Artificial Sequence: Synthetic peptide
ADCFLSRPTEKT

SEQ ID NO: 2 - protein, 12 aa, Artificial Sequence
  Description of Artificial Sequence: Synthetic peptide
VDCALSRPTEKT

SEQ ID NO: 3 - protein, 12 aa, Artificial Sequence
  Description of Artificial Sequence: Synthetic peptide
VDCFLARPTEKT

SEQ ID NO: 4 - protein, 12 aa, Artificial Sequence
  Description of Artificial Sequence: Synthetic peptide
VDCFLSRPAEKT

SEQ ID NO: 5 - protein, 12 aa, Artificial Sequence
  Description of Artificial Sequence: Synthetic peptide
VDCFLSRPTEKA

SEQ ID NO: 6 - protein, 10 aa, Artificial Sequence
  Description of Artificial Sequence: Synthetic peptide
CFLSRPTAKT

SEQ ID NO: 7 - protein, 10 aa, Artificial Sequence
  Description of Artificial Sequence: Synthetic peptide
CALSRPTEKT

SEQ ID NO: 8 - protein, 10 aa, Artificial Sequence
  Description of Artificial Sequence: Synthetic peptide
CFLARPTEKT

SEQ ID NO: 9 - protein, 10 aa, Artificial Sequence
  Description of Artificial Sequence: Synthetic peptide
CFLSRPAEKT

SEQ ID NO: 10 - protein, 10 aa, Artificial Sequence
  Description of Artificial Sequence: Synthetic peptide
CFLSRPTEKA

SEQ ID NO: 11 - protein, 12 aa, Artificial Sequence
  Description of Artificial Sequence: Synthetic peptide
VDCFISRPTEKT

SEQ ID NO: 12 - protein, 12 aa, Artificial Sequence
  Description of Artificial Sequence: Synthetic peptide
VDCFVSRPTEKT

SEQ ID NO: 13 - protein, 12 aa, Artificial Sequence
  Description of Artificial Sequence: Synthetic peptide
VNCYVSRPTEKN

SEQ ID NO: 14 - protein, 12 aa, Artificial Sequence
  Description of Artificial Sequence: Synthetic peptide
IDCFVSRPTEKT

SEQ ID NO: 15 - protein, 12 aa, Artificial Sequence
  Description of Artificial Sequence: Synthetic peptide
VDCFVSRPTEKT

SEQ ID NO: 16 - protein, 12 aa, Artificial Sequence
  Description of Artificial Sequence: Synthetic peptide
VDCFVSRPTEKT

SEQ ID NO: 17 - protein, 12 aa, Artificial Sequence
  Description of Artificial Sequence: Synthetic peptide
VDCFVSRPTEKT

SEQ ID NO: 18 - protein, 12 aa, Artificial Sequence
  Description of Artificial Sequence: Synthetic peptide
VDCYISRPTEKK

SEQ ID NO: 19 - protein, 12 aa, Artificial Sequence
  Description of Artificial Sequence: Synthetic peptide
VDCFISKPSEKN

SEQ ID NO: 20 - protein, 12 aa, Artificial Sequence
  Description of Artificial Sequence: Synthetic peptide
VDCYIARPTEKK

SEQ ID NO: 21 - protein, 12 aa, Artificial Sequence
  Description of Artificial Sequence: Synthetic peptide
VDCFISKPTEKT

SEQ ID NO: 22 - protein, 12 aa, Artificial Sequence
  Description of Artificial Sequence: Synthetic peptide
VDCFVSRPTEKT

SEQ ID NO: 23 - protein, 12 aa, Artificial Sequence
  Description of Artificial Sequence: Synthetic peptide
VDCFISRPTEKT

SEQ ID NO: 24 - protein, 12 aa, Artificial Sequence
  Description of Artificial Sequence: Synthetic peptide
VDCFVSRPTEKT

SEQ ID NO: 25 - protein, 12 aa, Artificial Sequence
  Description of Artificial Sequence: Synthetic peptide
VECYVSRPTEKT

SEQ ID NO: 26 - protein, 12 aa, Artificial Sequence
  Description of Artificial Sequence: Synthetic peptide
VDCFVSRPTEKT

SEQ ID NO: 27 - protein, 12 aa, Artificial Sequence
  Description of Artificial Sequence: Synthetic peptide
VDCYVSRPTEKS

SEQ ID NO: 28 - protein, 12 aa, Artificial Sequence
  Description of Artificial Sequence: Synthetic peptide
ITCNLSRPSEKT

SEQ ID NO: 29 - protein, 12 aa, Artificial Sequence
  Description of Artificial Sequence: Synthetic peptide
ITCNLSRPSEKT

SEQ ID NO: 30 - protein, 12 aa, Artificial Sequence
  Description of Artificial Sequence: Synthetic peptide
IDCFVSRPTEKT

SEQ ID NO: 31 - protein, 12 aa, Artificial Sequence
  Description of Artificial Sequence: Synthetic peptide
VDCFVSRPTEKT

SEQ ID NO: 32 - protein, 12 aa, Artificial Sequence
  Description of Artificial Sequence: Synthetic peptide
VDCFISRPTEKT

SEQ ID NO: 33 - protein, 12 aa, Artificial Sequence
  Description of Artificial Sequence: Synthetic peptide
ITCNLSRPSEKT

SEQ ID NO: 34 - protein, 12 aa, Artificial Sequence
  Description of Artificial Sequence: Synthetic peptide
VDCYVSRPTEKS

SEQ ID NO: 35 - protein, 12 aa, Artificial Sequence
  Description of Artificial Sequence: Synthetic peptide
VECYVSRPTEKT

SEQ ID NO: 36 - protein, 12 aa, Artificial Sequence
  Description of Artificial Sequence: Synthetic peptide
IDCFVSRPTEKT

SEQ ID NO: 37 - protein, 12 aa, Artificial Sequence
  Description of Artificial Sequence: Synthetic peptide
VDCYISRPTEKK

SEQ ID NO: 38 - protein, 12 aa, Artificial Sequence
  Description of Artificial Sequence: Synthetic peptide
VDCFISKPSEKN

SEQ ID NO: 39 - protein, 12 aa, Artificial Sequence
  Description of Artificial Sequence: Synthetic peptide
VDCYIARPTEKK

SEQ ID NO: 40 - protein, 12 aa, Artificial Sequence
  Description of Artificial Sequence: Synthetic peptide
VDCFISKPTEKT

SEQ ID NO: 41 - protein, 12 aa, Artificial Sequence
  Description of Artificial Sequence: Synthetic peptide
IDCFVSRPTEKT

SEQ ID NO: 42 - protein, 12 aa, Artificial Sequence
  Description of Artificial Sequence: Synthetic peptide
VNCYVSRPTEKN

SEQ ID NO: 43 - protein, 12 aa, Artificial Sequence
  Description of Artificial Sequence: Synthetic peptide
IDCFISRPTEKT

SEQ ID NO: 44 - protein, 13 aa, Artificial Sequence
  Description of Artificial Sequence: Synthetic peptide
VCYDKSFPISHVR

SEQ ID NO: 45 - protein, 12 aa, Artificial Sequence
  Description of Artificial Sequence: Synthetic peptide
VDCFLSRPTEKT

SEQ ID NO: 46 - protein, 26 aa, Artificial Sequence
  Description of Artificial Sequence: Synthetic peptide
  See specification as filed for detailed description of substitutions and preferred embodiments
VDCFLSRPTEKTGVCYDKSFPISHVR

SEQ ID NO: 47 - protein, 12 aa, Artificial Sequence
  Description of Artificial Sequence: Synthetic peptide
VDCFLSRPTEKT

SEQ ID NO: 48 - protein, 12 aa, Artificial Sequence
  Description of Artificial Sequence: Synthetic peptide
VNCYVSRPTEKN

SEQ ID NO: 49 - protein, 25 aa, Artificial Sequence
  Description of Artificial Sequence: Synthetic peptide
  See specification as filed for detailed description of substitutions and preferred embodiments
VDCFLSRPTEKTGVNCYVSRPTEKN

SEQ ID NO: 50 - protein, 12 aa, Artificial Sequence
  Description of Artificial Sequence: Synthetic peptide
VDCFVSRPTEKN

SEQ ID NO: 51 - protein, 12 aa, Artificial Sequence
  Description of Artificial Sequence: Synthetic peptide
VDCFLSRPTEKN

SEQ ID NO: 52 - protein, 12 aa, Artificial Sequence
  Description of Artificial Sequence: Synthetic peptide
VDCFVSRPTEKT

SEQ ID NO: 53 - skipped

SEQ ID NO: 54 - protein, 16 aa, Unknown
  Description of Unknown: ANTP peptide
RQPKIWFPNRRKPWKK

SEQ ID NO: 55 - protein, 11 aa, Human immunodeficiency virus
GRKKRRQRPPQ

SEQ ID NO: 56 - protein, 26 aa, Unknown
  Description of Unknown: Transportan peptide
GWTLNSAGYLLGKINKALAALAKKIL

SEQ ID NO: 57 - protein, 21 aa, Unknown
  Description of Unknown: Buforin II peptide
TRSSRAGLQFPVGRVHRLLRK

SEQ ID NO: 58 - protein, 9 aa, Human immunodeficiency virus
RKKRRQRRR

SEQ ID NO: 59 - protein, 16 aa, Unknown
  Description of Unknown: Penetratin peptide
RQIKIWFQNRRMKWKK

SEQ ID NO: 60 - protein, 18 aa, Unknown
  Description of Unknown: MAP peptide
KLALKLALKALKAALKLA

SEQ ID NO: 61 - protein, 16 aa, Unknown
  Description of Unknown: K-FGF peptide
AAVALLPAVLLALLAP

SEQ ID NO: 62 - protein, 10 aa, Unknown
  Description of Unknown: Ku70 peptide
VPMLKPMLKE

SEQ ID NO: 63 - protein, 28 aa, Unknown
  Description of Unknown: Prion peptide
MANLGYWLLALFVTMWTDVGLCKKRPKP

SEQ ID NO: 64 - protein, 18 aa, Unknown
  Description of Unknown: pVEC peptide
LLIILRRRIRKQAHAHSK

SEQ ID NO: 65 - protein, 21 aa, Unknown
  Description of Unknown: Pep-1 peptide
KETWWETWWTEWSQPKKKRRV

SEQ ID NO: 66 - protein, 18 aa, Unknown
  Description of Unknown: SynB1 peptide
RGGRLSYSRRRFSTSTGR

SEQ ID NO: 67 - protein, 15 aa, Unknown
  Description of Unknown: Pep-7 peptide
SDLWEMMMVSLACQY

SEQ ID NO: 68 - protein, 12 aa, Unknown
  Description of Unknown: HN-1 peptide
TSPLNIHNGQKL

SEQ ID NO: 69 - protein, 16 aa, Unknown
  Description of Unknown: plsl peptide
RVIRVWFQNKRCKDKK

SEQ ID NO: 70 - protein, 27 aa, Unknown
  Description of Unknown: MGB Peptide P-beta peptide
GALFLGFLGAAGSTMGAWSQPKKKRKV

SEQ ID NO: 71 - protein, 27 aa, Unknown
  Description of Unknown: MGB Peptide P-alpha peptide
GALFLAFLAAALSLMGLWSQPKKKRRV

SEQ ID NO: 72 - protein, 11 aa, Artificial Sequence
  Description of Artificial Sequence: Synthetic peptide
VDCLSRPTEKT

SEQ ID NO: 73 - protein, 12 aa, Artificial Sequence
  Description of Artificial Sequence: Synthetic peptide
  MOD_RES (4)..(4): Phe, or an amino acid selected from the group consisting of Ala, Ile, Leu, Val, dehydroalanine, aminoisobutyric acid, and/or Trp, Tyr, Met, Ile, Leu, and/or Ser, Asn, Cys, Gln, Thr, Asp, Glu, Arg, His, Lys, Pro, and/or Nva or Nle, and/or tert-butylglycine,
  MOD_RES (4)..(4): continued from above; phenylglycine, 7-azatryptophan, 4-fluorophenylalanine, N-methyl-methionine, N-methyl-valine, N-methyl-alanine, sarcosine, N-methyl-tert- butylglycine, N-methyl-leucine, N-methyl-phenylglycine, N-methyl-isoleucine,
  MOD_RES (4)..(4): continued from above; N-methyl-tryptophan, N-methyl-7- azatryptophan, N-methyl-phenylalanine, N-methyl-4- fluorophenylalanine, N-methyl-threonine, N-methyl-tyrosine, N-methyl-valine, and N-methyl-lysine,
  MOD_RES (4)..(4): continued from above; and/or a D-enantiomer of any of the foregoing
  MOD_RES (9)..(9): Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle
  MOD_RES (10)..(10): Glu, Ala, dehydroalanine, aminoisobutyric acid, Asp, Lys, Gln, Gly, His, Arg, Asn, Met, Pro, Ser, Thr, Nva or Nle
  MOD_RES (12)..(12): Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle
  See specification as filed for detailed description of substitutions and preferred embodiments
VDCXLSRPXXKX

SEQ ID NO: 74 - protein, 12 aa, Artificial Sequence
  Description of Artificial Sequence: Synthetic peptide
  MOD_RES (1)..(1): Val, Ala or an amino acid selected from the group consisting of Ile, Leu, Met, Phe, Thr, Cys, Tyr, dehydroalanine, aminoisobutyric acid, Nva, Nle, tert-butylglycine, phenylglycine, 7-azatryptophan, 4-fluorophenylalanine, N-methyl-methionine,
  MOD_RES (1)..(1): continued from above; N-methyl-valine, N-methyl-alanine, sarcosine, N-methyl-tert-butylglycine, N-methyl-leucine, N-methyl- phenylglycine, N-methyl-isoleucine, N-methyl-tryptophan, N-methyl- 7-azatryptophan, N-methyl- phenylalanine,
  MOD_RES (1)..(1): continued from above; N-methyl-4- fluorophenylalanine, N-methyl- threonine, N-methyl-tyrosine, N-methyl-valine, and N-methyl-lysine, and/or a D-enantiomer of any of the foregoing
  MOD_RES (4)..(4): Phe, or an amino acid selected from the group consisting of Ala, Ile, Leu, Val, dehydroalanine, aminoisobutyric acid, and/or Trp, Tyr, Met, Ile, Leu, and/or Ser, Asn, Cys, Gln, Thr, Asp, Glu, Arg, His, Lys, Pro, and/or Nva or Nle, and/or tert-butylglycine,
  MOD_RES (4)..(4): continued from above; phenylglycine, 7-azatryptophan, 4-fluorophenylalanine, N-methyl-methionine, N-methyl-valine, N-methyl-alanine, sarcosine, N-methyl-tert- butylglycine, N-methyl-leucine, N-methyl-phenylglycine, N-methyl-isoleucine,
  MOD_RES (4)..(4): continued from above; N-methyl-tryptophan, N-methyl-7- azatryptophan, N-methyl-phenylalanine, N-methyl-4- fluorophenylalanine, N-methyl-threonine, N-methyl-tyrosine, N-methyl-valine, and N-methyl-lysine,
  MOD_RES (4)..(4): continued from above; and/or a D-enantiomer of any of the foregoing
  MOD_RES (6)..(6): Ser, Ala or an amino acid selected from the group consisting of Asn, Thr, Cys, Gly, Asp, Glu, Lys, Gln, Gly, Pro, His or Arg
  MOD_RES (9)..(9): Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle
  MOD_RES (10)..(10): Glu, Ala, dehydroalanine, aminoisobutyric acid, Asp, Lys, Gln, Gly, His, Arg, Asn, Met, Pro, Ser, Thr, Nva or Nle
  MOD_RES (12)..(12): Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle
  See specification as filed for detailed description of substitutions and preferred embodiments
XDCXLXRPXXKX

SEQ ID NO: 75 - protein, 12 aa, Artificial Sequence
  Description of Artificial Sequence: Synthetic peptide
  MOD_RES (4)..(4): Phe, or an amino acid selected from the group consisting of Ala, Ile, Leu, Val, dehydroalanine, aminoisobutyric acid, and/or Trp, Tyr, Met, Ile, Leu, and/or Ser, Asn, Cys, Gln, Thr, Asp, Glu, Arg, His, Lys, Pro, and/or Nva or Nle, and/or tert-butylglycine,
  MOD_RES (4)..(4): continued from above; phenylglycine, 7-azatryptophan, 4-fluorophenylalanine, N-methyl-methionine, N-methyl-valine, N-methyl-alanine, sarcosine, N-methyl-tert- butylglycine, N-methyl-leucine, N-methyl-phenylglycine, N-methyl-isoleucine,
  MOD_RES (4)..(4): continued from above; N-methyl-tryptophan, N-methyl-7- azatryptophan, N-methyl-phenylalanine, N-methyl-4- fluorophenylalanine, N-methyl-threonine, N-methyl-tyrosine, N-methyl-valine, and N-methyl-lysine,
  MOD_RES (4)..(4): continued from above; and/or a D-enantiomer of any of the foregoing
  MOD_RES (9)..(9): Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle
  MOD_RES (10)..(10): Glu, Ala, dehydroalanine, aminoisobutyric acid, Asp, Lys, Gln, Gly, His, Arg, Asn, Met, Pro, Ser, Thr, Nva or Nle
  MOD_RES (12)..(12): Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle
IDCXISRPXXKX

SEQ ID NO: 76 - protein, 12 aa, Artificial Sequence
  Description of Artificial Sequence: Synthetic peptide
  MOD_RES (1)..(1): Ile, Val, Leu, Ala, Met, and/or Phe or an amino acid selected from the group consisting of Ile, Leu, Met, Phe, Thr, Cys, Tyr, dehydroalanine, aminoisobutyric acid, Nva, Nle, tert-butylglycine, phenylglycine, 7-azatryptophan,
  MOD_RES (1)..(1): continued from above; 4-fluorophenylalanine, N-methyl-methionine, N-methyl-valine, N-methyl-alanine, sarcosine, N-methyl-tert- butylglycine, N-methyl-leucine, N-methyl- phenylglycine, N-methyl- isoleucine, N-methyl-tryptophan, N-methyl-7-azatryptophan,
  MOD_RES (1)..(1): continued from above; N-methyl-phenylalanine, N-methyl-4- fluorophenylalanine, N-methyl-threonine, N-methyl- tyrosine, N-methyl-valine, and N-methyl-lysine, and/or a D-enantiomer of any of the foregoing
  MOD_RES (4)..(4): Phe, or an amino acid selected from the group consisting of Ala, Ile, Leu, Val, dehydroalanine, aminoisobutyric acid, and/or Trp, Tyr, Met, Ile, Leu, and/or Ser, Asn, Cys, Gln, Thr, Asp, Glu, Arg, His, Lys, Pro, and/or Nva or Nle, and/or tert-butylglycine,
  MOD_RES (4)..(4): continued from above; phenylglycine, 7-azatryptophan, 4-fluorophenylalanine, N-methyl-methionine, N-methyl-valine, N-methyl-alanine, sarcosine, N-methyl-tert- butylglycine, N-methyl-leucine, N-methyl-phenylglycine, N-methyl- isoleucine,
  MOD_RES (4)..(4): continued from above; N-methyl-tryptophan, N-methyl-7- azatryptophan, N-methyl-phenylalanine, N-methyl-4- fluorophenylalanine, N-methyl-threonine, N-methyl-tyrosine, N-methyl-valine, and N-methyl-lysine,
  MOD_RES (4)..(4): continued from above; and/or a D-enantiomer of any of the foregoing
  MOD_RES (6)..(6): Ser, Ala or an amino acid selected from the group consisting of Asn, Thr, Cys, Gly, Asp, Glu, Lys, Gln, Gly, Pro, His or Arg
  MOD_RES (9)..(9): Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle
  MOD_RES (10)..(10): Glu, Ala, dehydroalanine, aminoisobutyric acid, Asp, Lys, Gln, Gly, His, Arg, Asn, Met, Pro, Ser, Thr, Nva or Nle
  MOD_RES (12)..(12): Thr, or an amino acid selected from the group consisting of Ser, Asn, Ala, Ile, Leu, Val, dehydroalanine, aminoisobutyric acid, Asp, Glu, Lys, Met, His, Pro, Gln, Arg, Ser, Nva or Nle
  See specification as filed for detailed description of substitutions and preferred embodiments
XDCXIXRPXXKX

SEQ ID NO: 77 - protein, 7 aa, Artificial Sequence
  Description of Artificial Sequence: Synthetic peptide
SRPTEKT

SEQ ID NO: 78 - protein, 12 aa, Artificial Sequence
  Description of Artificial Sequence: Synthetic peptide
VACFLSRPTEKT

SEQ ID NO: 79 - protein, 12 aa, Artificial Sequence
  Description of Artificial Sequence: Synthetic peptide
VDAFLSRPTEKT

SEQ ID NO: 80 - protein, 12 aa, Artificial Sequence
  Description of Artificial Sequence: Synthetic peptide
VDCFASRPTEKT

SEQ ID NO: 81 - protein, 12 aa, Artificial Sequence
  Description of Artificial Sequence: Synthetic peptide
VDCFLSAPTEKT

SEQ ID NO: 82 - protein, 12 aa, Artificial Sequence
  Description of Artificial Sequence: Synthetic peptide
VDCFLSRATEKT

SEQ ID NO: 83 - protein, 12 aa, Artificial Sequence
  Description of Artificial Sequence: Synthetic peptide
VDCFLSRPTAKT

SEQ ID NO: 84 - protein, 12 aa, Artificial Sequence
  Description of Artificial Sequence: Synthetic peptide
VDCFLSRPTEAT

SEQ ID NO: 85 - protein, 10 aa, Artificial Sequence
  Description of Artificial Sequence: Synthetic peptide
CFLSRPTEKT

SEQ ID NO: 86 - protein, 8 aa, Artificial Sequence
  Description of Artificial Sequence: Synthetic peptide
LSRPTEKT

SEQ ID NO: 87 - protein, 10 aa, Artificial Sequence
  Description of Artificial Sequence: Synthetic peptide
VDCFLSRPTE

SEQ ID NO: 88 - protein, 8 aa, Artificial Sequence
  Description of Artificial Sequence: Synthetic peptide
VDCFLSRP

SEQ ID NO: 89 - protein, 6 aa, Artificial Sequence
  Description of Artificial Sequence: Synthetic peptide
VDCFLS

SEQ ID NO: 90 - protein, 12 aa, Artificial Sequence
  Description of Artificial Sequence: Synthetic peptide
RFKPSLCTTDEV

SEQ ID NO: 91 - protein, 35 aa, Artificial Sequence
  Description of Artificial Sequence: Synthetic polypeptide
ESAWGDEQSAFRCNTQQPGCENVCYDKSFPISHVR

SEQ ID NO: 92 - protein, 34 aa, Artificial Sequence
  Description of Artificial Sequence: Synthetic polypeptide
QWYIYGFSLSAVYTCKRDPCPHQVDCFLSRPTEK

SEQ ID NO: 93 - protein, 17 aa, Artificial Sequence
  Description of Artificial Sequence: Synthetic peptide
ESAWGDEQSAFRCNTQQ

SEQ ID NO: 94 - protein, 18 aa, Artificial Sequence
  Description of Artificial Sequence: Synthetic peptide
PGCENVCYDKSFPISHVR

SEQ ID NO: 95 - protein, 17 aa, Artificial Sequence
  Description of Artificial Sequence: Synthetic peptide
AFRCNTQQPGCENVCYD

SEQ ID NO: 96 - protein, 19 aa, Artificial Sequence
  Description of Artificial Sequence: Synthetic peptide
FLLIQWYIYGFSLSAVYTC

SEQ ID NO: 97 - protein, 20 aa, Artificial Sequence
  Description of Artificial Sequence: Synthetic peptide
KRDPCPHQVDCFLSRPTEKT

SEQ ID NO: 98 - protein, 20 aa, Artificial Sequence
  Description of Artificial Sequence: Synthetic peptide
GFSLSAVYTCKRDPCPHQVD